(19) United States
(12) Patent Application Publication
(10) Pub. No.: US 2018/0215788 A1
(43) Pub. Date: Aug. 2, 2018

(54) Compounds and Compositions for the Treatment of Ophthalmic Disorders

(71) Applicant: Shire Human Genetic Therapies, Inc., Lexington, MA (US)

(72) Inventors: Anneli Savinainen, Lexington, MA (US); Mohannad Shawer, Lexington, MA (US)

(21) Appl. No.: 15/886,751

(22) Filed: Feb. 1, 2018

Related U.S. Application Data
(60) Provisional application No. 62/453,153, filed on Feb. 1, 2017.

(51) Int. Cl. C07K 7/06; A61K 38/08; A61K 31/216; A61K 9/00; A61K 31/5377; A61P 27/02

(52) U.S. Cl. CPC C07K 7/06; A61K 38/08; A61P 27/02; A61K 9/0048; A61K 31/5377; A61K 31/216

(57) ABSTRACT

Described herein are methods and compositions featuring a first compound that is a linear peptidic NPR-B agonist and a second compound that is a prostaglandin agonist or a β-adrenergic antagonist, which are useful in the treatment and/or prevention of ophthalmic disorders such as glaucoma.

### CROSS-REFERENCE TO RELATED APPLICATIONS

This application claims priority to U.S. Provisional Patent Application No. 62/453,153, filed on Feb. 1, 2017, the entirety of which is hereby incorporated by reference.

### SEQUENCE LISTING

The instant application contains a Sequence Listing which has been submitted electronically in ASCII format and is hereby incorporated by reference in its entirety. Said ASCII copy, created on Jan. 31, 2018, is named “SHR-1253WO_Sequence Listing.txt” and is 1,015 bytes in size.

### FIELD OF INVENTION

The present invention generally relates to novel compounds and compositions which are useful in the treatment and/or prevention of disorders such as ophthalmic disorders.

### BACKGROUND OF THE INVENTION

Glaucoma is the second leading cause of blindness world-wide (Thylefors and Negrel 1994, Bull World Health Organ. 72:323-326). Open-angle glaucoma (OAG) and angle-closure glaucoma combined represent the second leading cause of blindness worldwide (Quigley and Broman, 2006 Br J Ophthalmol. 90:262-267). The prevalence of angle-closure glaucoma or open-angle glaucoma varies among different sub-populations (see, e.g., Foster et al. 2000, Arch Ophthalmol. 118:1105-11; Leske et al. 2007, Ophthalmic Epidemiol. 14:166-172). Glaucoma is a progressive disease in which the risk of vision loss increases with disease duration. In light of an aging population world-wide, the impact of this blinding disorder can be expected to increase in the future.

The disease state referred to as glaucoma is a family of diseases characterized by a permanent loss of visual function due to irreversible damage to the optic nerve. More specifically, glaucoma results in optic neuropathy leading to the loss of retinal ganglion cell (RGC) function followed by apoptotic cell death and a progressive increase in vision loss. Morphologically or functionally distinct types of glaucoma are typically characterized by elevated intraocular pressure (IOP), which is considered to be an important risk factor of the pathological course of the disease.

Disruption of normal aqueous outflow leading to elevated IOP is integral to glaucoma pathophysiology. Ocular hypertension is a condition wherein IOP is elevated but no apparent loss of visual function has occurred; such patients are considered to be at high risk for the eventual development of the visual loss associated with glaucoma. Some patients with glaucomatous field loss have relatively low IOPs. These so called normotension or low tension glaucoma patients can also benefit from agents that lower and control IOP.

Glaucoma is typically identified by changes in IOP, visual field deficits and/or fundus changes at the optic disk. Elevated IOP, found in most glaucoma patients, is a result of morphological and biochemical changes in the trabecular meshwork (TM), an aqueous humor filtering tissue located at the iris-cornea angle of the eye. As glaucoma progresses, there is a loss of TM cells and a buildup of extracellular products which inhibit the normal aqueous humor outflow resulting in IOP elevation. In addition to elevated IOP, other factors, such as genetic defects, may lead to mechanical distortion of the optic nerve head (ONH) ultimately resulting in ONH cupping and loss of RGC and their axons. The exact mechanism of this pathological process is currently unknown. It has been suggested that lowering the IOP of patients diagnosed with glaucoma by at least 20-30% will decrease the progressive worsening of the disease by 50-60% (Quigley 2005 Ophthalmology 112:1642-1643). Without proper diagnosis and treatment, glaucoma can progress to total irreversible blindness.

Initially, most open-angle glaucoma patients are managed with one or more of a wide variety of topical ocular or oral hypotensive medications that act to increase aqueous fluid outflow and/or decrease aqueous fluid production, or with surgical procedures such as laser trabeculoplasty and filtration surgery. Treatment regimens currently available for patients exhibiting elevated IOP, regardless of cause, typically include the topical application, from once daily to multiple times per day, of one or multiple eyedrops or pills containing a small molecule IOP-lowering compound. Also, pills that decrease the amount of aqueous humor created can be given between two and four times daily. Glaucoma medications typically prescribed include cholinergic agonists, adrenergic agonists, beta adrenergic blockers, carbonic anhydrase inhibitors and prostaglandin analogs.

### SUMMARY OF THE INVENTION

Although these classes of medications are effective in controlling IOP, each of them has certain limitations in efficacy and untoward effects. For example, beta adrenergic blockers do not lower IOP at night; many glaucoma patients do not respond to a particular drug class; and a majority of glaucoma patients require the use of a combination of drugs. In addition, many of the drugs cause local irritation of the eye, such as burning, stinging, itching, tearing, conjunctival hyperemia, foreign body sensation, blurred vision, and eye pain. Some occasionally induce systemic side effects. Hence, there is a genuine and continuous need for novel and improved glaucoma medications.

The presence of natriuretic peptides and their receptors in ocular tissues, especially those involved in the regulation of intraocular pressure (IOP), have been demonstrated. In the past 20 years, the natriuretic peptides have been shown to be highly effective as IOP-lowering agents. Administration of natriuretic peptides using intravitreal injection, subconjunctival injection, intracameral injection, or systemic administration has been studied for effecting reduction of IOP. While natriuretic peptides have favorable therapeutic effects, topical administration has posed challenges due to the inability of these peptides to penetrate the cornea. Recently, novel linear peptide NPR-B agonists having improved bioavailability (as compared to isolated or synthesized natriuretic peptides) have been identified and developed as useful for the treatment of natriuretic peptide-mediated disorders, such as ophthalmic disorders.

A second class of therapeutic agents suitable for the treatment of ophthalmic disorders are the prostaglandin agonists, which can be used to reduce IOP. Four prostaglandin agonists—bimatoprost, latanoprost, tafluprost, and travoprost—have been approved by the United States Food & Drug Administration as glaucoma medications. Such compounds can be administered topically as a dilute ophthalmic solution.

A still further class of therapeutic agents suitable for the treatment of ophthalmic disorders include β-adrenergic antagonists, which also can be used to reduce IOP. Exemplary β-adrenergic antagonists include betaxolol, carteolol, levobunolol, metipranolol, and timolol (e.g., timolol maleate or timolol hemihydrate).

The present invention is in part based on the finding that a combination of linear peptide NPR-B agonists and a second agent that is a prostaglandin agonist or a β-adrenergic antagonist can result in improved (e.g., synergistic) therapeutic effects such as increased reduction of IOP. The compounds, compositions, and methods described herein can be useful for treatment or prevention of disorders such as ophthalmic disorders as described herein.

In a first aspect, the invention features a method of treating an ophthalmic disease in a patient, said method comprising co-administering to said patient an effective amount of a first compound that is a linear peptidic NPR-B agonist; and a second compound that is a prostaglandin agonist or a β-adrenergic antagonist. In embodiments, an ophthalmic disease is associated with elevated intraocular pressure or ocular hypertension. In embodiments, an ophthalmic disease is glaucoma, elevated intraocular pressure or ocular hypertension. In embodiments, a second compound is a prostaglandin agonist. In embodiments, a second compound is a β-adrenergic antagonist.

In another aspect, the invention features method of lowering intraocular pressure in a patient in need thereof, said method comprising co-administering to said patient an effective amount of a first compound that is a linear peptidic NPR-B agonist; and a second compound that is a prostaglandin agonist or a β-adrenergic antagonist. In embodiments, a second compound is a prostaglandin agonist. In embodiments, a second compound is a β-adrenergic antagonist.

In a further aspect, the invention features a method of treating glaucoma in a patient in need thereof, said method comprising co-administering to said patient an effective amount of: a first compound that is a linear peptidic NPR-B agonist; and a second compound that is a prostaglandin agonist or a β-adrenergic antagonist. In embodiments, a glaucoma is primary open angle glaucoma, angle closure glaucoma, normal tension glaucoma, congenital glaucoma, neovascular glaucoma, steroid-induced glaucoma, or glaucoma related to ocular trauma. In embodiments, a glaucoma is primary open angle glaucoma. In embodiments, a second compound is a prostaglandin agonist. In embodiments, a second compound is a β-adrenergic antagonist.

In embodiments, a first compound is a compound of formula (B-1),

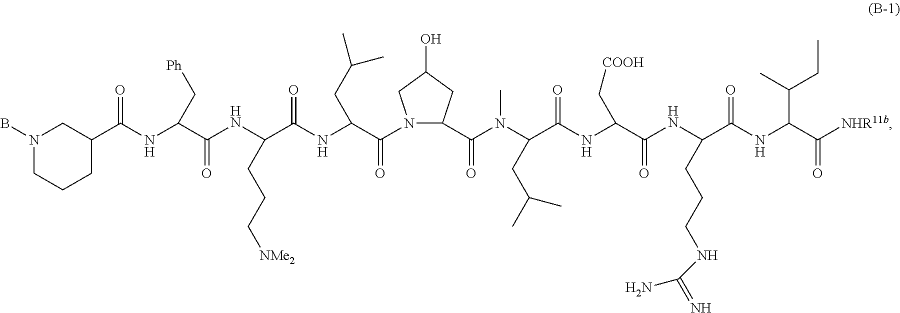

or a pharmaceutically acceptable salt thereof, wherein

B is selected from the group consisting of R^{b1}— and R^{b2}—C(O)—;

R^{b1} is selected from the group consisting of C₆-C₁₀ alkyl and C₆-C₁₀ alkyl substituted by NR^{b4}R^{b5};

R^{b2} is selected from the group consisting of C₆-C₁₀ alkyl and C₆-C₁₀ alkyl substituted by NR^{b4}R^{b5};

R^{b4} and R^{b5} are, independently, selected from the group consisting of H and C₁-C₄ alkyl; and

R^{11b} is selected from the group consisting of H, C₁-C₈ alkyl, C₄-C₈ cycloalkyl, C₂-C₁₂ bicycloalkyl, and C₁-C₄ alkyl-C₄-C₈ cycloalkyl.

In embodiments, a first compound is Occ-Sni-Phe-orn(Me2)-Leu-Hyp-Nml-Asp-Arg-Ile-NH₂ (SEQ ID NO:1), or a pharmaceutically acceptable salt thereof.

In embodiments, a first compound is

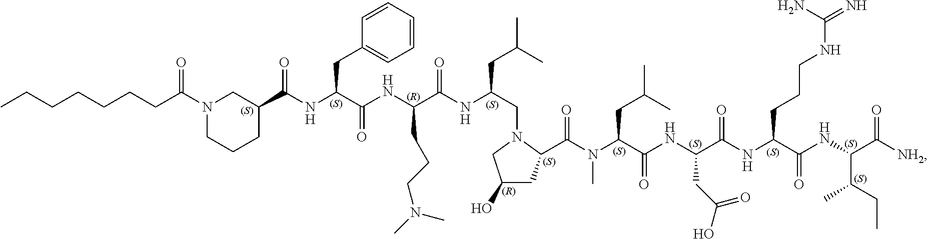

or a pharmaceutically acceptable salt thereof.

In embodiments, a second compound is a prostaglandin agonist.

In embodiments, a second compound has a structure according to the following formula,

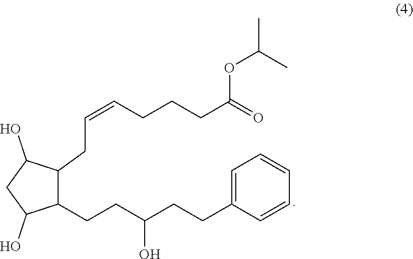 [SMILES: CC(C)OC(=O)CCC/C=C\CC1C(O)CC(O)C1CCC(O)CCc1ccccc1]

In embodiments, a second compound is latanoprost, bimatoprost, travoprost, or tafluprost.

In embodiments, a second compound is latanoprost. In embodiments, a second compound is bimatoprost. In embodiments, a second compound is travoprost. In embodiments, a second compound is tafluprost.

In embodiments, a second compound has a structure according to the following formula,

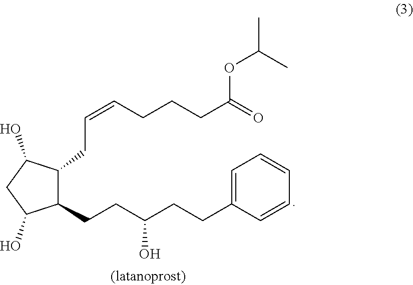 [SMILES: CC(C)OC(=O)CCC/C=C\C[C@@H]1[C@@H](CC[C@@H](O)CCc2ccccc2)[C@H](O)C[C@@H]1O]

In embodiments, a second compound is a β-adrenergic antagonist.

In embodiments, a second compound is betaxolol, carteolol, levobunolol, metipranolol, or timolol (e.g., timolol maleate or timolol hemihydrate). In embodiments, a second compound is betaxolol. In embodiments, a second compound is carteolol. In embodiments, a second compound is levobunolol. In embodiments, a second compound is metipranolol. In embodiments, a second compound is timolol (e.g., timolol maleate or timolol hemihydrate).

In embodiments, a first compound and a second compound are administered simultaneously.

In embodiments, a first compound and a second compound are administered contemporaneously.

In embodiments, a first compound is administered before, during, or after administration of a second compound. In embodiments, a first compound is administered before administration of a second compound. In embodiments, a first compound is administered during administration of a second compound. In embodiments, a first compound is administered after administration of a second compound.

In embodiments, a first compound and a second compound are administered as a single composition (e.g., a single pharmaceutical composition).

In embodiments, a single composition comprises: a first compound in an amount that is about 0.01% (w/w) to about 0.75% (w/w); a second compound in an amount that is about 0.001% (w/w) to about 0.05% (w/w); and a pharmaceutically acceptable excipient.

In embodiments, a single composition comprises: a first compound in an amount that is about 0.01% (w/w) to about 0.5% (w/w). In embodiments, a single composition comprises a first compound in an amount that is about 0.01% (w/w) to about 0.20% (w/w). In embodiments, a single composition comprises a first compound in an amount that is about 0.01% (w/w) to about 0.15% (w/w). In embodiments, a single composition comprises a first compound in an amount that is about 0.01% (w/w). In embodiments, a single composition comprises a first compound in an amount that is about 0.02% (w/w). In embodiments, a single composition comprises a first compound in an amount that is about 0.03% (w/w). In embodiments, a single composition comprises a first compound in an amount that is about 0.04% (w/w). In embodiments, a single composition comprises a first compound in an amount that is about 0.05% (w/w). In embodiments, a single composition comprises a first compound in an amount that is about 0.06% (w/w). In embodiments, a single composition comprises a first compound in an amount that is about 0.07% (w/w). In embodiments, a single composition comprises a first compound in an amount that is about 0.08% (w/w). In embodiments, a single composition comprises a first compound in an amount that is about 0.09% (w/w). In embodiments, a single composition comprises a first compound in an amount that is about 0.1% (w/w). In embodiments, a single composition comprises a first compound in an amount that is about 0.11% (w/w). In embodiments, a single composition comprises a first compound in an amount that is about 0.12% (w/w). In embodiments, a single composition comprises a first compound in an amount that is about 0.13% (w/w). In embodiments, a single composition comprises a first compound in an amount that is about 0.14% (w/w). In embodiments, a single composition comprises a first compound in an amount that is about 0.15% (w/w). In embodiments, a single composition comprises a first compound in an amount that is about 0.2% (w/w). In embodiments, a single composition comprises a first compound in an amount that is about 0.25% (w/w). In embodiments, a first compound is a compound of formula (B-1) such as a compound of SEQ ID NO:1.

In embodiments, a single composition comprises a second compound in an amount that is about 0.0001% (w/w) to about 0.1% (w/w). In embodiments, a single composition comprises a second compound in an amount that is about 0.001% (w/w) to about 0.1% (w/w). In embodiments, a single composition comprises a second compound in an amount that is about 0.001% (w/w) to about 0.05% (w/w).

In embodiments, a single composition comprises a second compound in an amount that is about 0.0005% (w/w) to about 0.05% (w/w). In embodiments, a single composition comprises a second compound in an amount that is about 0.0001% (w/w) to about 0.01% (w/w). In embodiments, a single composition comprises a second compound in an amount that is about 0.001% (w/w) to about 0.01% (w/w). In embodiments, a single composition comprises a second compound in an amount that is about 0.001% (w/w) to about 0.005% (w/w). In embodiments, a single composition comprises a second compound in an amount that is about 0.0001% (w/w). In embodiments, a single composition comprises a second compound in an amount that is about 0.0002% (w/w). In embodiments, a single composition comprises a second compound in an amount that is about 0.0003% (w/w). In embodiments, a single composition comprises a second compound in an amount that is about 0.0004% (w/w). In embodiments, a single composition comprises a second compound in an amount that is about 0.0005% (w/w). In embodiments, a second compound is a prostaglandin agonist. In embodiments, a second compound is latanoprost. In embodiments, a second compound is travoprost. In embodiments, a second compound is bimatoprost.

In embodiments, a single composition comprises a second compound in an amount that is about 0.001% (w/w) to about 0.01% (w/w). In embodiments, a single composition comprises a second compound in an amount that is about 0.002% (w/w). In embodiments, a single composition comprises a second compound in an amount that is about 0.003% (w/w). In embodiments, a single composition comprises a second compound in an amount that is about 0.004% (w/w). In embodiments, a single composition comprises a second compound in an amount that is about 0.005% (w/w). In embodiments, a single composition comprises a second compound in an amount that is about 0.006% (w/w). In embodiments, a single composition comprises a second compound in an amount that is about 0.007% (w/w). In embodiments, a single composition comprises a second compound in an amount that is about 0.008% (w/w). In embodiments, a single composition comprises a second compound in an amount that is about 0.009% (w/w). In embodiments, a single composition comprises a second compound in an amount that is about 0.01% (w/w). In embodiments, a second compound is a prostaglandin agonist. In embodiments, a second compound is latanoprost. In embodiments, a second compound is travoprost. In embodiments, a second compound is travoprost. In embodiments, a second compound is bimatoprost.

In embodiments, a single composition comprises a second compound in an amount that is about 0.01% (w/w) to about 0.1% (w/w). In embodiments, a single composition comprises a second compound in an amount that is about 0.01% (w/w). In embodiments, a single composition comprises a second compound in an amount that is about 0.02% (w/w). In embodiments, a single composition comprises a second compound in an amount that is about 0.03% (w/w). In embodiments, a single composition comprises a second compound in an amount that is about 0.04% (w/w). In embodiments, a single composition comprises a second compound in an amount that is about 0.05% (w/w). In embodiments, a single composition comprises a second compound in an amount that is about 0.06% (w/w). In embodiments, a single composition comprises a second compound in an amount that is about 0.07% (w/w). In embodiments, a single composition comprises a second compound in an amount that is about 0.08% (w/w). In embodiments, a single composition comprises a second compound in an amount that is about 0.09% (w/w). In embodiments, a single composition comprises a second compound in an amount that is about 0.1% (w/w). In embodiments, a second compound is a prostaglandin agonist. In embodiments, a second compound is latanoprost. In embodiments, a second compound is travoprost. In embodiments, a second compound is travoprost. In embodiments, a second compound is bimatoprost.

In embodiments, a single composition comprises a first compound in an amount that is about 0.01% (w/w) to about 0.2% (w/w); and a second compound in an amount that is about 0.001% (w/w) to about 0.1% (w/w). In embodiments, a first compound is a compound of formula (B-1) such as a compound of SEQ ID NO:1. In embodiments, a second compound is latanoprost. In embodiments, a second compound is travoprost. In embodiments, a second compound is bimatoprost.

In embodiments, a single composition comprises a first compound that is a compound of formula (B-1) such as a compound of SEQ ID NO:1 (e.g., in an amount that is about 0.01% (w/w) to about 0.15% (w/w) such as about 0.03% (w/w), about 0.10% (w/w), or about 0.60% (w/w)), and a second compound that is latanoprost (e.g., in an amount that is about 0.001% (w/w) to about 0.01% (w/w) such as about 0.005% (w/w)).

In embodiments, a single composition comprises a first compound that is a compound of formula (B-1) such as a compound of SEQ ID NO:1 (e.g., in an amount that is about 0.01% (w/w) to about 0.15% (w/w) such as about 0.03% (w/w), about 0.10% (w/w), or about 0.60% (w/w)), and a second compound that is travoprost (e.g., in an amount that is about 0.001% (w/w) to about 0.01% (w/w) such as about 0.005% (w/w)).

In embodiments, a single composition comprises a first compound that is a compound of formula (B-1) such as a compound of SEQ ID NO:1 (e.g., in an amount that is about 0.01% (w/w) to about 0.15% (w/w) such as about 0.03% (w/w), about 0.10% (w/w), or about 0.60% (w/w)), and a second compound that is bimatoprost (e.g., in an amount that is about 0.01% (w/w) to about 0.1% (w/w) such as about 0.03% (w/w)).

In embodiments, a single composition comprises at least one excipient that is a viscosity enhancing agent, a surfactant, a buffering agent, or a tonicity agent, or any combination thereof. In embodiments, a single composition comprises one or more excipients selected from the group consisting of a viscosity enhancing agent, a surfactant, a buffering agent, and a tonicity agent. In embodiments, a viscosity enhancing agent is present in an amount that is about 0.05% (w/w) to about 0.75% (w/w). In embodiments, a viscosity enhancing agent is present in an amount that is about 0.25% (w/w) to about 0.6% (w/w). In embodiments, a surfactant is present in an amount that is about 0.001% (w/w) to about 0.2% (w/w). In embodiments, a surfactant is present in an amount that is about 0.005% (w/w) to about 0.1% (w/w). In embodiments, a buffering agent is present in an amount that is about 0.01% (w/w) to about 0.75% (w/w). In embodiments, a buffering agent is present in an amount that is about 0.1% (w/w) to about 0.6% (w/w). In embodiments, a tonicity agent is present in an amount that is about 0.1% (w/w) to about 0.9% (w/w). In embodiments, a tonicity agent is present in an amount that is about 0.3% (w/w) to about 0.8% (w/w).

In embodiments, a single composition comprises hydroxypropyl methylcellulose (HPMC), polysorbate 80, a phosphate buffering agent, and sodium chloride. In embodiments the viscosity enhancing agent is hydroxypropyl methylcellulose (HPMC). In embodiments, HPMC is present in an amount that is about 0.05% (w/w) to about 0.75% (w/w). In embodiments, HPMC is present in an amount that is about 0.25% (w/w) to about 0.6% (w/w). In embodiments, HPMC is present in an amount that is 0.5 (w/w). In embodiments, the surfactant is polysorbate 80. In embodiments, polysorbate 80 is present in an amount that is about 0.001% (w/w) to about 0.2% (w/w). In embodiments, polysorbate 80 is present in an amount that is about 0.005% (w/w) to about 0.1% (w/w). In embodiments, polysorbate 80 is present in an amount that is 0.05 (w/w). In embodiments, the buffering agent is dibasic sodium phosphate. In embodiments, dibasic sodium phosphate is present in an amount that is about 0.01% (w/w) to about 0.75% (w/w). In embodiments, dibasic sodium phosphate is present in an amount that is about 0.1% (w/w) to about 0.6% (w/w). In embodiments, dibasic sodium phosphate is present in an amount that is 0.2 (w/w). In embodiments, the tonicity agent is sodium chloride (NaCl). In embodiments, NaCl is present in an amount that is about 0.1% (w/w) to about 0.9% (w/w). In embodiments, NaCl is present in an amount that is about 0.3% (w/w) to about 0.8% (w/w). In embodiments, NaCl is present in an amount that is 0.75 (w/w).

In embodiments, a single composition has a pH of about 6.8 to about 7.2. In embodiments, a single composition has a pH of about 6.9 to about 7.1. In embodiments, a single composition has a pH of about 6.8. In embodiments, a single composition has a pH of about 6.9. In embodiments, a single composition has a pH of about 7.0. In embodiments, a single composition has a pH of about 7.1. In embodiments, a single composition has a pH of about 7.2.

In embodiments, a first compound and a second compound are administered as two separate compositions; that is, a first composition comprises a first compound, and a second composition comprises a second compound. In embodiments, a composition is a pharmaceutical composition. In embodiments, a first composition is a pharmaceutical composition. In embodiments, a second composition is a pharmaceutical composition. In embodiments, both a first composition and a second composition are pharmaceutical compositions.

In embodiments, a first composition comprising a first compound is administered before a second composition comprising a second compound.

In embodiments, a first composition comprising a first compound is administered with a second composition comprising a second compound.

In embodiments, a first composition comprising a first compound is administered after a second composition comprising a second compound.

In embodiments, a first composition comprises: a first compound in an amount that is about 0.01% (w/w) to about 0.75% (w/w); a second compound in an amount that is about 0.001% (w/w) to about 0.05% (w/w); and a pharmaceutically acceptable excipient.

In embodiments, a first composition comprises: a first compound in an amount that is about 0.01% (w/w) to about 0.5% (w/w). In embodiments, a first composition comprises a first compound in an amount that is about 0.01% (w/w) to about 0.20% (w/w). In embodiments, a first composition comprises a first compound in an amount that is about 0.01% (w/w) to about 0.15% (w/w). In embodiments, a first composition comprises a first compound in an amount that is about 0.01% (w/w). In embodiments, a first composition comprises a first compound in an amount that is about 0.02% (w/w). In embodiments, a first composition comprises a first compound in an amount that is about 0.03% (w/w). In embodiments, a first composition comprises a first compound in an amount that is about 0.04% (w/w). In embodiments, a first composition comprises a first compound in an amount that is about 0.05% (w/w). In embodiments, a first composition comprises a first compound in an amount that is about 0.06% (w/w). In embodiments, a first composition comprises a first compound in an amount that is about 0.07% (w/w). In embodiments, a first composition comprises a first compound in an amount that is about 0.08% (w/w). In embodiments, a first composition comprises a first compound in an amount that is about 0.09% (w/w). In embodiments, a first composition comprises a first compound in an amount that is about 0.1% (w/w). In embodiments, a first composition comprises a first compound in an amount that is about 0.11% (w/w). In embodiments, a first composition comprises a first compound in an amount that is about 0.12% (w/w). In embodiments, a first composition comprises a first compound in an amount that is about 0.13% (w/w). In embodiments, a first composition comprises a first compound in an amount that is about 0.14% (w/w). In embodiments, a first composition comprises a first compound in an amount that is about 0.15% (w/w). In embodiments, a first composition comprises a first compound in an amount that is about 0.2% (w/w). In embodiments, a first composition comprises a first compound in an amount that is about 0.25% (w/w). In embodiments, a first compound is a compound of formula (B-1) such as a compound of SEQ ID NO:1.

In embodiments, a first composition comprises a first compound in an amount that is about 0.01% (w/w) to about 0.2% (w/w). In embodiments, a first composition comprises a first compound in an amount that is about 0.01% (w/w) to about 0.15% (w/w). In embodiments, a first composition comprises a first compound in an amount that is about 0.03% (w/w), about 0.10% (w/w), or about 0.60% (w/w). In embodiments, a first compound is a compound of formula (B-1) such as a compound of SEQ ID NO:1. In embodiments, a first composition comprises a first compound in an amount that is about 0.03% (w/w). In embodiments, a first composition comprises a first compound in an amount that is about 0.10% (w/w). In embodiments, a first composition comprises a first compound in an amount that is about 0.60% (w/w). In embodiments, a first compound is a compound of formula (B-1) such as a compound of SEQ ID NO:1.

In embodiments, a first composition further comprises at least one excipient that is a viscosity enhancing agent, a preservative, a chelating agent, a surfactant, a buffering agent, or a tonicity agent, or any combination thereof. In embodiments, a first composition comprises one or more excipients selected from the group consisting of a viscosity enhancing agent, a preservative, a chelating agent, a surfactant, a buffering agent, and a tonicity agent. In embodiments, a viscosity enhancing agent is present in an amount that is about 0.05% (w/w) to about 0.75% (w/w). In embodiments, a viscosity enhancing agent is present in an amount that is about 0.25% (w/w) to about 0.6% (w/w). In embodiments, a surfactant is present in an amount that is about 0.001% (w/w) to about 0.2% (w/w). In embodiments, a surfactant is present in an amount that is about 0.005% (w/w) to about 0.1% (w/w). In embodiments, a buffering agent is present in an amount that is about 0.01% (w/w) to about 0.75% (w/w). In embodiments a buffering agent is present in an amount that is about 0.1% (w/w) to about 0.6% (w/w). In embodiments, a tonicity agent is present in an amount that is about 0.1% (w/w) to about 0.9% (w/w). In embodiments, a tonicity agent is present in an amount that is about 0.3% (w/w) to about 0.8% (w/w). In embodiments, a preservative is present in an amount that is about 0.001% (w/w) to about 0.2% (w/w). In embodiments, a preservative is present in an amount that is about 0.005% (w/w) to about 0.1% (w/w). In embodiments, a chelating agent is present in an amount that is about 0.001% (w/w) to about 0.2% (w/w). In embodiments, a chelating agent is present in an amount that is about 0.005% (w/w) to about 0.1% (w/w).

In embodiments, a first composition comprises hydroxypropyl methylcellulose (HPMC), one or more tetraalkylammonium salts, ethylenediaminetetraacetic acid (EDTA), polysorbate 80, a phosphate buffering agent, and sodium chloride. In embodiments the viscosity enhancing agent is hydroxypropyl methylcellulose (HPMC). In embodiments, HPMC is present in an amount that is about 0.05% (w/w) to about 0.75% (w/w). In embodiments, HPMC is present in an amount that is about 0.25% (w/w) to about 0.6% (w/w). In embodiments, HPMC is present in an amount that is 0.5 (w/w). In embodiments, the surfactant is polysorbate 80. In embodiments, polysorbate 80 is present in an amount that is about 0.001% (w/w) to about 0.2% (w/w). In embodiments, polysorbate 80 is present in an amount that is about 0.005% (w/w) to about 0.1% (w/w). In embodiments, polysorbate 80 is present in an amount that is 0.05 (w/w). In embodiments, the buffering agent is dibasic sodium phosphate. In embodiments, dibasic sodium phosphate is present in an amount that is about 0.01% (w/w) to about 0.75% (w/w). In embodiments dibasic sodium phosphate is present in an amount that is about 0.1% (w/w) to about 0.6% (w/w). In embodiments, dibasic sodium phosphate is present in an amount that is 0.2 (w/w). In embodiments, the tonicity agent is sodium chloride (NaCl). In embodiments, NaCl is present in an amount that is about 0.1% (w/w) to about 0.9% (w/w). In embodiments, NaCl is present in an amount that is about 0.3% (w/w) to about 0.8% (w/w). In embodiments, NaCl is present in an amount that is 0.75 (w/w). In embodiments, the preservative is benzalkonium chloride (BAK). In embodiments, BAK is present in an amount that is about 0.001% (w/w) to about 0.2% (w/w). In embodiments, BAK is present in an amount that is about 0.005% (w/w) to about 0.1% (w/w). In embodiments, BAK is present in an amount that is 0.01 (w/w). In embodiments the chelating agent is ethylenediaminetetraacetic acid (EDTA). In embodiments, EDTA is present in an amount that is about 0.001% (w/w) to about 0.2% (w/w). In embodiments, EDTA is present in an amount that is about 0.005% (w/w) to about 0.1% (w/w). In embodiments, EDTA is present in an amount that is 0.01 (w/w).

In embodiments, a first composition has a pH of about 7.2 to about 7.6. In embodiments, a first composition has a pH of about 6.8 to about 7.6. In embodiments, a single composition has a pH of about 6.8. In embodiments, a single composition has a pH of about 6.9. In embodiments, a single composition has a pH of about 7.0. In embodiments, a single composition has a pH of about 7.1. In embodiments, a single composition has a pH of about 7.2. In embodiments, a single composition has a pH of about 7.3. In embodiments, a single composition has a pH of about 7.4. In embodiments, a single composition has a pH of about 7.5. In embodiments, a single composition has a pH of about 7.6.

In embodiments, a second composition comprises said second compound in an amount that is about 0.001% (w/w) to about 0.05% (w/w). In embodiments, a second composition comprises said second compound in an amount that is about 0.005% (w/w) to about 0.05% (w/w). In embodiments, a second composition comprises said second compound in an amount that is about 0.01% (w/w) to about 0.05% (w/w). In embodiments, a second composition comprises said second compound in an amount that is about 0.01% (w/w). In embodiments, a second composition comprises said second compound in an amount that is about 0.02% (w/w). In embodiments, a second composition comprises said second compound in an amount that is about 0.03% (w/w). In embodiments, a second composition comprises said second compound in an amount that is about 0.04% (w/w). In embodiments, a second composition comprises said second compound in an amount that is about 0.05% (w/w).

In embodiments, a second composition comprises a second compound in an amount that is about 0.0001% (w/w) to about 0.1% (w/w). In embodiments, a second composition comprises a second compound in an amount that is about 0.001% (w/w) to about 0.1% (w/w). In embodiments, a second composition comprises a second compound in an amount that is about 0.001% (w/w) to about 0.05% (w/w).

In embodiments, a second composition comprises a second compound in an amount that is about 0.0005% (w/w) to about 0.05% (w/w). In embodiments, a second composition comprises a second compound in an amount that is about 0.0001% (w/w) to about 0.01% (w/w). In embodiments, a second composition comprises a second compound in an amount that is about 0.001% (w/w) to about 0.01% (w/w). In embodiments, a second composition comprises a second compound in an amount that is about 0.001% (w/w) to about 0.005% (w/w). In embodiments, a second composition comprises a second compound in an amount that is about 0.0001% (w/w). In embodiments, a second composition comprises a second compound in an amount that is about 0.0002% (w/w). In embodiments, a second composition comprises a second compound in an amount that is about 0.0003% (w/w). In embodiments, a second composition comprises a second compound in an amount that is about 0.0004% (w/w). In embodiments, a second composition comprises a second compound in an amount that is about 0.0005% (w/w). In embodiments, a second compound is a prostaglandin agonist. In embodiments, a second compound is latanoprost. In embodiments, a second compound is travoprost. In embodiments, a second compound is bimatoprost.

In embodiments, a second composition comprises a second compound in an amount that is about 0.001% (w/w) to about 0.01% (w/w). In embodiments, a second composition comprises a second compound in an amount that is about 0.002% (w/w). In embodiments, a second composition comprises a second compound in an amount that is about 0.003% (w/w). In embodiments, a second composition comprises a second compound in an amount that is about 0.004% (w/w). In embodiments, a second composition comprises a second compound in an amount that is about 0.005% (w/w). In embodiments, a second composition comprises a second compound in an amount that is about 0.006% (w/w). In embodiments, a second composition comprises a second compound in an amount that is about 0.007% (w/w). In embodiments, a second composition comprises a second compound in an amount that is about 0.008% (w/w). In embodiments, a second composition comprises a second compound in an amount that is about 0.009% (w/w). In embodiments, a second composition comprises a second compound in an amount that is about 0.01% (w/w). In embodiments, a second compound is a prostaglandin agonist. In embodiments, a second compound is latanoprost. In embodiments, a second compound is travoprost. In embodiments, a second compound is travoprost. In embodiments, a second compound is bimatoprost.

In embodiments, a second composition comprises TRAVATAN Z®.

In embodiments, a second composition comprises a second compound in an amount that is about 0.01% (w/w) to about 0.1% (w/w). In embodiments, a second composition comprises a second compound in an amount that is about 0.01% (w/w). In embodiments, a second composition comprises a second compound in an amount that is about 0.02% (w/w). In embodiments, a second composition comprises a second compound in an amount that is about 0.03% (w/w). In embodiments, a second composition comprises a second compound in an amount that is about 0.04% (w/w). In embodiments, a second composition comprises a second compound in an amount that is about 0.05% (w/w). In embodiments, a second composition comprises a second compound in an amount that is about 0.06% (w/w). In embodiments, a second composition comprises a second compound in an amount that is about 0.07% (w/w). In embodiments, a second composition comprises a second compound in an amount that is about 0.08% (w/w). In embodiments, a second composition comprises a second compound in an amount that is about 0.09% (w/w). In embodiments, a second composition comprises a second compound in an amount that is about 0.1% (w/w). In embodiments, a second compound is a prostaglandin agonist. In embodiments, a second compound is latanoprost. In embodiments, a second compound is travoprost. In embodiments, a second compound is travoprost. In embodiments, a second compound is bimatoprost.

In embodiments, a second composition comprises a second compound in an amount that is about 0.001% (w/w) to about 0.01% (w/w). In embodiments, a second composition comprises a second compound in an amount that is about 0.002% (w/w). In embodiments, a second composition comprises a second compound in an amount that is about 0.003% (w/w). In embodiments, a second composition comprises a second compound in an amount that is about 0.004% (w/w). In embodiments, a second composition comprises a second compound in an amount that is about 0.005% (w/w). In embodiments, a second composition comprises a second compound in an amount that is about 0.006% (w/w). In embodiments, a second composition comprises a second compound in an amount that is about 0.007% (w/w). In embodiments, a second composition comprises a second compound in an amount that is about 0.008% (w/w). In embodiments, a second composition comprises a second compound in an amount that is about 0.009% (w/w). In embodiments, a second composition comprises a second compound in an amount that is about 0.01% (w/w). In embodiments, a second compound is a β-adrenergic antagonist. In embodiments, a second compound is timolol. In embodiments, a second compound is timolol maleate.

In embodiments, a second composition comprises TIMOPTIC®.

In embodiments, a second composition further comprises at least one excipient that is a preservative, a buffering agent, or a tonicity agent, or any combination thereof. In embodiments, a preservative is present in an amount that is about 0.001% (w/w) to about 0.2% (w/w). In embodiments, a preservative is present in an amount that is about 0.005% (w/w) to about 0.1% (w/w). In embodiments, a buffering agent is present in an amount that is about 0.01% (w/w) to about 0.75% (w/w). In embodiments a buffering agent is present in an amount that is about 0.1% (w/w) to about 0.6% (w/w). In embodiments, a tonicity agent is present in an amount that is about 0.1% (w/w) to about 0.9% (w/w). In embodiments, a tonicity agent is present in an amount that is about 0.3% (w/w) to about 0.8% (w/w).

In embodiments, a second composition further comprises one or more tetraalkylammonium salts, a phosphate buffering agent, and sodium chloride. In embodiments, the buffering agent is sodium phosphate. In embodiments, sodium phosphate is present in an amount that is about 0.01% (w/w) to about 0.75% (w/w). In embodiments, sodium phosphate is present in an amount that is about 0.1% (w/w) to about 0.6% (w/w). In embodiments, the tonicity agent is sodium chloride (NaCl). In embodiments, NaCl is present in an amount that is about 0.1% (w/w) to about 0.9% (w/w). In embodiments, NaCl is present in an amount that is about 0.3% (w/w) to about 0.8% (w/w). In embodiments, the preservative is benzalkonium chloride (BAK). In embodiments, BAK is present in an amount that is about 0.001% (w/w) to about 0.2% (w/w). In embodiments, BAK is present in an amount that is about 0.005% (w/w) to about 0.1% (w/w). In embodiments, BAK is present in an amount that is 0.02 (w/w).

In embodiments, a second composition has a pH of about 6.8 to about 7.6. In embodiments, a second composition has a pH of about 6.8. In embodiments, a second composition has a pH of about 6.9. In embodiments, a second composition has a pH of about 7.0. In embodiments, a second composition has a pH of about 7.1. In embodiments, a second composition has a pH of about 7.2. In embodiments, a second composition has a pH of about 7.3. In embodiments, a second composition has a pH of about 7.4. In embodiments, a second composition has a pH of about 7.5. In embodiments, a second composition has a pH of about 7.6.

In embodiments, a first composition comprises a first compound in an amount that is about 0.01% (w/w) to about 0.2% (w/w); and a second composition comprises a second compound in an amount that is about 0.001% (w/w) to about 0.1% (w/w). In embodiments, a first compound is a compound of formula (B-1) such as a compound of SEQ ID NO:1. In embodiments, a second compound is latanoprost. In embodiments, a second compound is travoprost. In embodiments, a second compound is bimatoprost.

In embodiments, a first composition comprises a first compound that is a compound of formula (B-1) such as a compound of SEQ ID NO:1 (e.g., in an amount that is about 0.01% (w/w) to about 0.15% (w/w) such as about 0.03% (w/w), about 0.10% (w/w), or about 0.60% (w/w)), and a second composition comprises a second compound that is latanoprost (e.g., in an amount that is about 0.001% (w/w) to about 0.01% (w/w) such as about 0.005% (w/w)).

In embodiments, a first composition comprises a first compound that is a compound of formula (B-1) such as a compound of SEQ ID NO:1 (e.g., in an amount that is about 0.01% (w/w) to about 0.15% (w/w) such as about 0.03% (w/w), about 0.10% (w/w), or about 0.60% (w/w)), and a second composition comprises a second compound that is travoprost (e.g., in an amount that is about 0.001% (w/w) to about 0.01% (w/w) such as about 0.005% (w/w)).

In embodiments, a first composition comprises a first compound that is a compound of formula (B-1) such as a compound of SEQ ID NO:1 (e.g., in an amount that is about 0.01% (w/w) to about 0.15% (w/w) such as about 0.03% (w/w), about 0.10% (w/w), or about 0.60% (w/w)), and a second composition comprises a second compound that is bimatoprost (e.g., in an amount that is about 0.01% (w/w) to about 0.1% (w/w) such as about 0.03% (w/w)).

In embodiments, a first compound and/or a second compound is topically administered. In embodiments, a single composition comprising a first compound and a second compound is topically administered. In embodiments, a first composition comprising a first compound and a second composition comprising a compound are topically administered.

In embodiments, a first compound and/or a second compound is administered using a medical implant. In embodiments, a first compound and a second compound are both administered using a medical implant.

In embodiments, a medical implant is an intraocular implant.

In embodiments, a medical implant is injectable.

In embodiments, a medical implant is biodegradable.

In embodiments, a medical implant comprises a hydrogel.

In a further aspect, the invention features a pharmaceutical composition comprising: a first compound that is a linear peptidic NPR-B agonist; a second compound that is a prostaglandin agonist or a β-adrenergic antagonist; and a pharmaceutically acceptable excipient.

In embodiments, a first compound is a compound of formula (B-1),

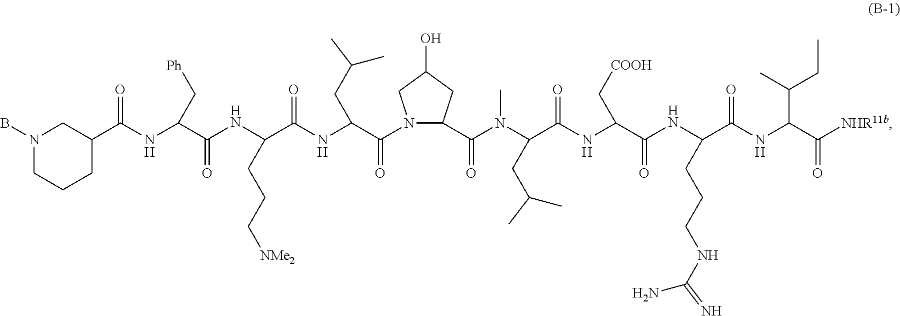

or a pharmaceutically acceptable salt thereof, wherein

B is selected from the group consisting of R^{b1}— and R^{b2}—C(O)—;

R^{b1} is selected from the group consisting of C₆-C₁₀ alkyl and C₆-C₁₀ alkyl substituted by NR^{b4}R^{b5};

R^{b2} is selected from the group consisting of C₆-C₁₀ alkyl and C₆-C₁₀ alkyl substituted by NR^{b4}R^{b5};

R^{b4} and R^{b5} are, independently, selected from the group consisting of H and C₁-C₄ alkyl; and

R^{11b} is selected from the group consisting of H, C₁-C₈ alkyl, C₄-C₈ cycloalkyl, C₂-C₁₂ bicycloalkyl, and C₁-C₄ alkyl-C₄-C₈ cycloalkyl.

In embodiments, a first compound is Occ-Sni-Phe-orn(Me2)-Leu-Hyp-Nml-Asp-Arg-Ile-NH₂ (SEQ ID NO:1), or a pharmaceutically acceptable salt thereof.

In embodiments, a first compound is

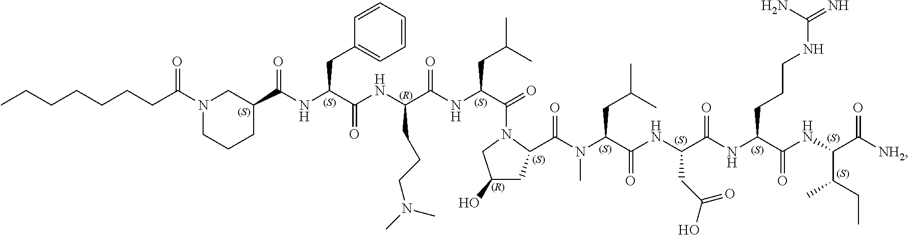

or a pharmaceutically acceptable salt thereof.

In embodiments, a second compound is a prostaglandin agonist.

In embodiments, a second compound has a structure according to the following formula,

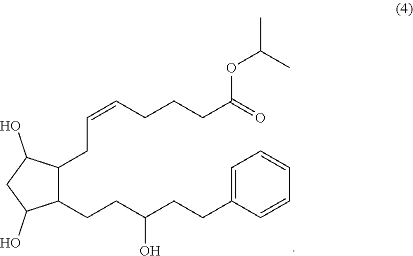 [SMILES: CC(C)OC(=O)CCC/C=C\CC1C(O)CC(O)C1CCC(O)CCc1ccccc1]

In embodiments, a second compound is latanoprost, bimatoprost, travoprost, or tafluprost.

In embodiments, a second compound has a structure according to the following formula,

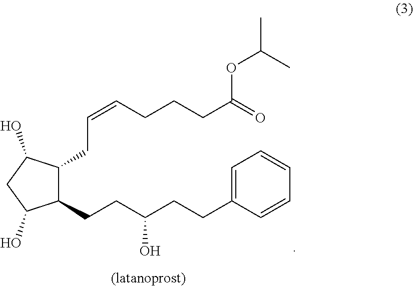 [SMILES: CC(C)OC(=O)CCC/C=C\C[C@@H]1[C@@H](CC[C@@H](O)CCc2ccccc2)[C@H](O)C[C@@H]1O]

In embodiments, a second compound is a β-adrenergic antagonist.

In embodiments, a second compound is betaxolol, carteolol, levobunolol, metipranolol, or timolol (e.g., timolol maleate or timolol hemihydrate). In embodiments, a second compound is betaxolol. In embodiments, a second compound is carteolol. In embodiments, a second compound is levobunolol. In embodiments, a second compound is metipranolol. In embodiments, a second compound is timolol (e.g., timolol maleate or timolol hemihydrate).

In embodiments, a first compound, or a pharmaceutically acceptable salt thereof, is present in an amount that is about 0.001% (w/w) to about 2.000% (w/w), about 0.010% (w/w) to about 1.500% (w/w), about 0.010% (w/w) to about 1.000%, about 0.010% (w/w) to about 0.900% (w/w), about 0.010% (w/w) to about 0.800% (w/w), about 0.010% (w/w) to about 0.700% (w/w), about 0.010% (w/w) to about 0.600% (w/w), about 0.010% (w/w) to about 0.500% (w/w), about 0.010% (w/w) to about 0.400% (w/w), about 0.010% (w/w) to about 0.300% (w/w), about 0.010% (w/w) to about 0.200% (w/w), about 0.010% (w/w) to about 0.15% (w/w), about 0.020% (w/w) to about 0.200% (w/w), about 0.020% (w/w) to about 0.150% (w/w), about 0.050% (w/w) to about 0.200% (w/w), or about 0.050% (w/w) to about 0.150% (w/w).

In embodiments, a second compound is present in an amount that is about 0.001% (w/w) to about 0.500% (w/w), about 0.001% (w/w) to about 0.250% (w/w), about 0.001% to about 0.100%, about 0.001% to about 0.090%, about 0.001% to about 0.075%, about 0.001% to about 0.050%, or about 0.001% to about 0.010%.

In embodiments, a first compound, or a pharmaceutically acceptable salt thereof, is present in an amount that is about 0.01% (w/w) to about 0.20% (w/w), 0.05% (w/w) to about 0.20% (w/w), or about 0.05% (w/w) to about 0.15% (w/w); and a second compound is present in an amount that is about 0.001% (w/w) to about 0.100% (w/w), or about 0.001% (w/w) to about 0.010% (w/w). In embodiments, a first compound, or a pharmaceutically acceptable salt thereof, is present in an amount that is about 0.03% (w/w). In embodiments, a first compound, or a pharmaceutically acceptable salt thereof, is present in an amount that is about 0.1% (w/w).

In embodiments, a first compound, or a pharmaceutically acceptable salt thereof, is present in an amount that is about 0.05% (w/w) to about 0.15% (w/w); and a second compound is present in an amount that is about 0.001% (w/w) to about 0.010% (w/w). In embodiments, a first compound, or a pharmaceutically acceptable salt thereof, is present in an amount that is about 0.1% (w/w).

In embodiments, the relative (w/w) ratio of said first compound to said second compound is about 100 to about 1; about 50 to about 1; about 40 to about 1; about 30 to about 1; about 20 to about 1; about 10 to about 1; about 1 to about 1; about 1 to about 10; or about 1 to about 20.

In embodiments, the relative (w/w) ratio of said first compound to said second compound is about 40 to about 1; about 35 to about 1; about 30 to about 1; about 25 to about 1; about 20 to about 1; about 15 to about 1; about 10 to about 1; or about 5 to about 1.

In embodiments, the relative (w/w) ratio of said first compound to said second compound is about 30 to about 1; about 25 to about 1; about 20 to about 1; or about 15 to about 1.

In embodiments, a composition (e.g., a pharmaceutical composition) is formulated for ophthalmic use.

In embodiments, a composition (e.g., a pharmaceutical composition) is formulated for topical administration.

In embodiments, a composition (e.g., a pharmaceutical composition) is formulated for administration using an injectable implant.

In embodiments, an injectable implant comprises a hydrogel.

In embodiments, a hydrogel comprises one or more cellulosic polymers.

In embodiments, a hydrogel comprises HPMC, HPC, polycarbophil, or xanthan gum.

In embodiments, an injectable implant comprises a biodegradable polymer.

In embodiments, an injectable implant comprises a polyester biodegradable polymer.

In embodiments, an injectable implant comprises a polyester biodegradable polymer that is PLA or PLGA.

In embodiments, an injectable implant releases the pharmaceutical composition for a period that is about 3-24 months, about 3-18 months, about 3-15 months, about 3-12 months, about 3-6 months, about 4-15 months, about 4-12 months, about 5-15 months, about 5-12 months, about 6-15 months, or about 6-12 months.

In embodiments, a composition (e.g., a pharmaceutical composition) comprises a pharmaceutically acceptable excipient selected from tonicity agents, viscosity enhancing agents, buffering agents, pH adjusting agents, surfactants, preservatives, chelating agents, and combinations thereof. In embodiments, a pharmaceutical composition comprises one or more excipients selected from the group consisting of a viscosity enhancing agent, a preservative, a chelating agent, a surfactant, a buffering agent, and a tonicity agent. In embodiments, a pharmaceutical composition comprises a viscosity enhancing agent, a preservative, a chelating agent, a surfactant, a buffering agent, and a tonicity agent. In embodiments, a pharmaceutical composition comprises hydroxypropyl methylcellulose (HPMC), one or more tetraalkylammonium salts, ethylenediaminetetraacetic acid (EDTA), polysorbate 80, a phosphate buffering agent, and sodium chloride.

In embodiments, a pharmaceutical composition has a pH that is about 6.5 to about 7.5, about 6.5 to about 7.0, about 7.0 to about 7.5, about 6.45 to about 6.55, about 6.55 to about 6.65, about 6.65 to about 6.75, about 6.75 to about 6.85, about 6.85 to about 6.95, about 6.95 to about 7.05, about 7.05 to about 7.15, about 7.15 to about 7.25, about 7.25 to about 7.35, about 7.35 to about 7.45, or about 7.45 to about 7.55.

In another aspect, the invention features a method of treating an ophthalmic disease in a patient, said method comprising administering to said patient an effective amount of any pharmaceutical composition described herein (e.g., a pharmaceutical composition comprising a first compound that is a linear peptidic NPR-B agonist; a second compound that is a prostaglandin agonist; and a pharmaceutically acceptable excipient).

In another aspect, the invention features a method of lowering intraocular pressure in a patient in need thereof, said method comprising administering to said patient an effective amount of any pharmaceutical composition described herein (e.g., a pharmaceutical composition comprising a first compound that is a linear peptidic NPR-B agonist; a second compound that is a prostaglandin agonist; and a pharmaceutically acceptable excipient).

In another aspect, the invention features a method of treating glaucoma in a patient in need thereof, said method comprising administering to said patient an effective amount of any pharmaceutical composition described herein (e.g., a pharmaceutical composition comprising a first compound that is a linear peptidic NPR-B agonist; a second compound that is a prostaglandin agonist; and a pharmaceutically acceptable excipient).

In embodiments, administering occurs one, two, three, or four times daily.

In embodiments, administering occurs once daily.

In a further aspect, the invention features a compound of formula (I),

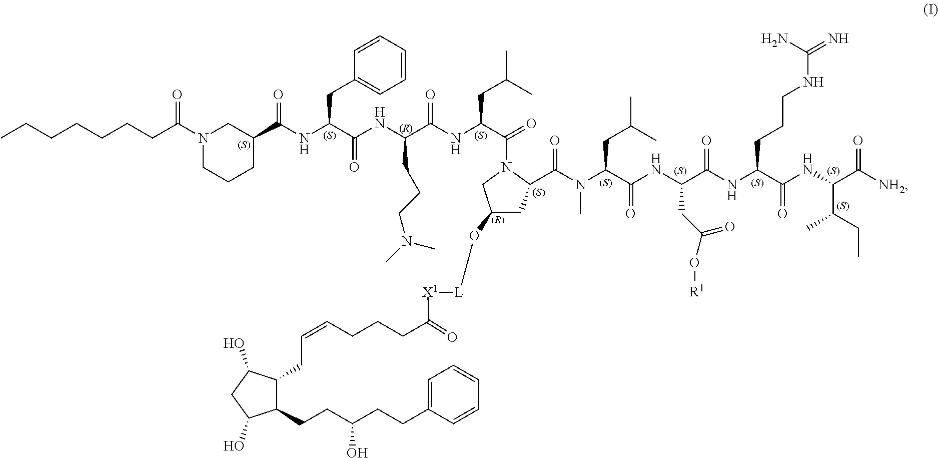

or a pharmaceutically acceptable salt thereof, wherein

X¹ is a covalent bond, —O—, —S—, or —NR^{X1}—,

R¹ is H, optionally substituted C₁-C₁₂ alkyl, or optionally substituted C₂-C₁₆ aralkyl;

R^{X1} is H or optionally substituted C₁-C₁₂ alkyl;

L represents a linker that is a covalent bond, optionally substituted C₁-C₁₂ alkylene, or optionally substituted 2- to 12-membered heteroalkylene, or

L represents a linker having the structure -(L¹)-L²-(L³)-, wherein
each of L¹ and L³ is independently a covalent bond, optionally substituted C₁-C₆ alkylene, or optionally substituted 2- to 6-membered heteroalkylene; and
L² is optionally substituted C₆-C₁₀ arylene or optionally substituted 5- to 10-membered heteroarylene.

In embodiments, R¹ is H, methyl, ethyl, isopropyl, n-propyl, tert-butyl, or benzyl.

In embodiments, R¹ is H.

In embodiments, X¹ is —O—.

In embodiments, linker L is optionally substituted C₁-C₁₂ alkylene.

In embodiments, linker L is optionally substituted 2- to 12-membered heteroalkylene.

In embodiments, linker L comprises one or more ethylene glycol substructures.

In embodiments, linker L comprises a substructure that is —CH₂CH₂—O—CH₂CH₂— or —CH₂CH₂—O—CH₂CH₂—O—CH₂CH₂—.

In embodiments, linker L comprises a substituent group that is an oxo (═O) moiety.

In embodiments, linker L is —(CH₂)ₙ—C(═O)—, and wherein n is 2, 3, 4, or 5.

In embodiments, in linker L is —CH₂CH₂—O—CH₂CH₂—C(═O)—, —CH₂CH₂—O—CH₂CH₂—O—CH₂CH₂—C(═O)—, —CH₂CH₂—O—CH₂CH₂—O—C(═O)—, or —CH₂CH₂—O—CH₂CH₂—O—CH₂CH₂—O—C(═O)—.

In embodiments, the compound is of formula (II),

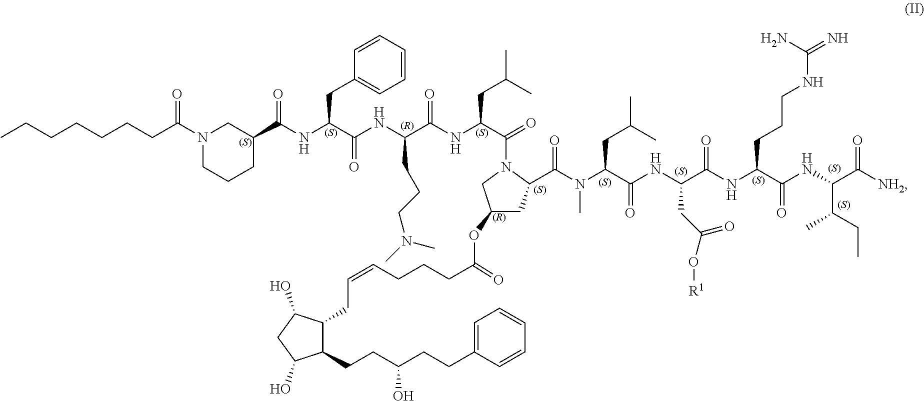

or a pharmaceutically acceptable salt thereof, wherein

R¹ is H, optionally substituted C₁-C₁₂ alkyl, or optionally substituted C₇-C₁₆ aralkyl.

In embodiments, R¹ is H, methyl, ethyl, isopropyl, n-propyl, tert-butyl, or benzyl.

In embodiments, R¹ is H.

In another aspect, the invention features a compound of formula (III),

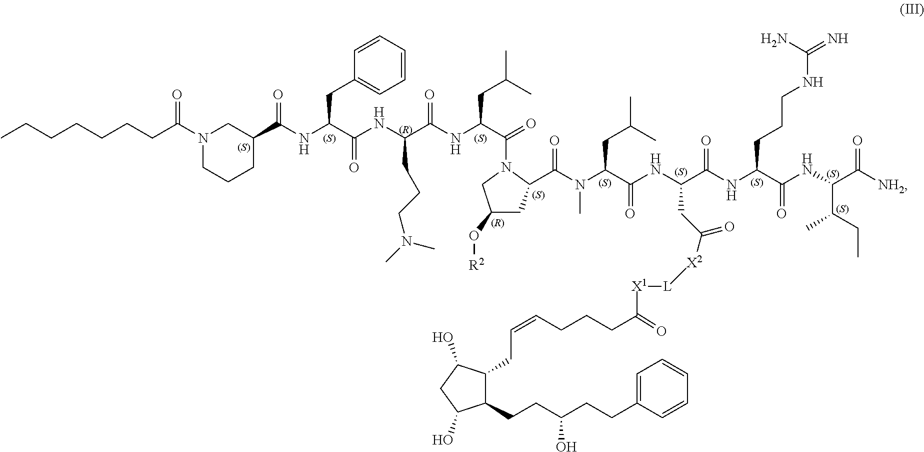

or a pharmaceutically acceptable salt thereof, wherein

R² is H, optionally substituted C₁-C₁₂ alkyl, or optionally substituted C₇-C₁₆ aralkyl;

X¹ is a covalent bond, —O—, —S—, or —NR^{X1}—,

X² is a covalent bond, —O—, —S—, or —NR^{X2}—,

each of R^{X1} and R^{X2} is independently H or optionally substituted C₁-C₁₂ alkyl;

L represents a linker that is a covalent bond, optionally substituted C₁-C₁₂ alkylene, or optionally substituted 2- to 12-membered heteroalkylene, or

L represents a linker having the structure -(L¹)-L²-(L³)-, wherein
each of L¹ and L³ is independently a covalent bond, optionally substituted C₁-C₆ alkylene, or optionally substituted 2- to 6-membered heteroalkylene; and
L² is optionally substituted C₆-C₁₀ arylene or optionally substituted 5- to 10-membered heteroarylene.

In embodiments, R² is H; or R² is acetyl, propionyl, pivaloyl, octanoyl, cyclopropanoyl, cyclohexanoyl, or benzyl, each of which is optionally substituted.

In embodiments, R² is H.

In embodiments, X¹ is —O—.

In embodiments, X² is —O—.

In embodiments, linker L is optionally substituted C₁-C₁₂ alkylene.

In embodiments, linker L is optionally substituted 2- to 12-membered heteroalkylene.

In embodiments, linker L comprises one or more ethylene glycol substructures.

In embodiments, linker L comprises a substructure that is —CH₂CH₂—O—CH₂CH₂— or —CH₂CH₂—O—CH₂CH₂—O—CH₂CH₂—.

In embodiments, linker L comprises a substituent group that is an oxo (═O) moiety.

In embodiments, linker L is —(CH₂)ₙ—C(═O)—, and wherein n is 2, 3, 4, or 5.

In embodiments, linker L is —CH₂CH₂—O—CH₂CH₂—C(═O)— or —CH₂CH₂—O—CH₂CH₂—O—CH₂CH₂—C(═O)—.

In embodiments, a compound is of formula (IV):

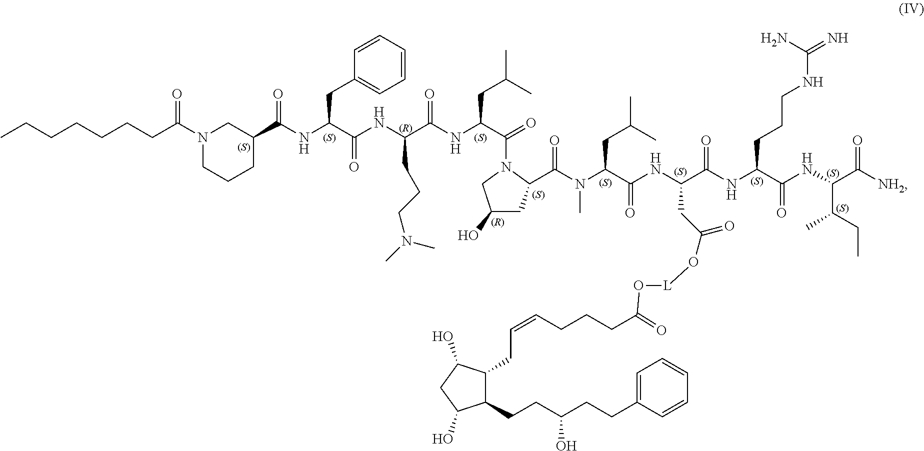

or a pharmaceutically acceptable salt thereof, wherein

L represents a linker that is optionally substituted C₁-C₁₂ alkylene or optionally substituted 2- to 12-membered heteroalkylene.

In embodiments, a linker L is —(CH₂)ₙ—, wherein n is 2, 3, 4, or 5.

In another aspect, the invention features a compound having the following structure,

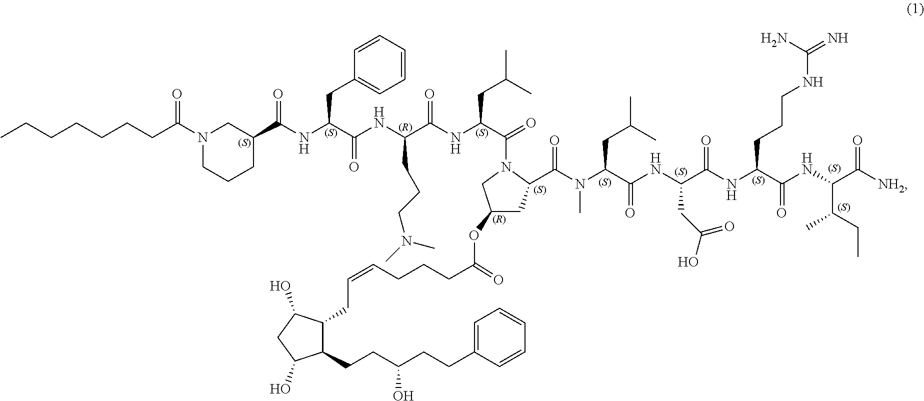

or a pharmaceutically acceptable salt thereof.

In another aspect, the invention features a pharmaceutical composition comprising any compound described herein (e.g., any compound of formula (I), (II), (III), or (IV) such as Compound (1)), and a pharmaceutically acceptable excipient.

In embodiments, a pharmaceutical composition comprises any compound described herein (e.g., any compound of formula (I), (II), (III), or (IV) such as Compound (1)) in an amount that is about 0.001% (w/w) to about 1.000% (w/w), about 0.001% (w/w) to about 0.500% (w/w), about 0.001% (w/w) to about 0.250% (w/w), about 0.001% to about 0.150%, about 0.001% to about 0.100%, about 0.001% to about 0.090%, about 0.001% to about 0.075%, about 0.001% to about 0.050%, or about 0.001% to about 0.010%.

In embodiments, a pharmaceutical composition comprises any compound described herein (e.g., any compound of formula (I), (II), (III), or (IV) such as Compound (1)) in an amount that is about 0.005% (w/w) to about 1.000% (w/w), about 0.005% (w/w) to about 0.500% (w/w), about 0.005% (w/w) to about 0.250% (w/w), about 0.005% to about 0.150%, about 0.005% to about 0.100%, about 0.005% to about 0.090%, about 0.005% to about 0.075%, about 0.005% to about 0.050%, or about 0.005% to about 0.010%.

In embodiments, a pharmaceutical composition comprises any compound described herein (e.g., any compound of formula (I), (II), (III), or (IV) such as Compound (1)) in an amount that is about 0.010% (w/w) to about 2.000% (w/w), about 0.010% (w/w) to about 1.500% (w/w), about 0.010% (w/w) to about 1.000% (w/w), about 0.010% (w/w) to about 0.900% (w/w), about 0.010% (w/w) to about 0.800% (w/w), about 0.010% (w/w) to about 0.700% (w/w), about 0.010% (w/w) to about 0.600% (w/w), about 0.010% (w/w) to about 0.500% (w/w), about 0.010% (w/w) to about 0.250% (w/w), about 0.010% to about 0.150%, about 0.010% to about 0.100%, about 0.010% to about 0.090%, about 0.010% to about 0.075%, or about 0.010% to about 0.050%.

In embodiments, a pharmaceutical composition comprises any compound described herein (e.g., any compound of formula (I), (II), (III), or (IV) such as Compound (1)) in an amount that is about 0.050% (w/w) to about 2.000% (w/w), about 0.050% (w/w) to about 1.500% (w/w), about 0.050% (w/w) to about 1.000% (w/w), about 0.050% (w/w) to about 0.500% (w/w), about 0.050% (w/w) to about 0.250% (w/w), about 0.050% (w/w) to about 0.200% (w/w), about 0.050% to about 0.150%, or about 0.050% to about 0.125%.

In embodiments, a pharmaceutical composition comprises any compound described herein (e.g., any compound of formula (I), (II), (III), or (IV) such as Compound (1)) in an amount that is about 0.075% (w/w) to about 2.000% (w/w), about 0.075% (w/w) to about 1.500% (w/w), about 0.075% (w/w) to about 1.250% (w/w), about 0.075% (w/w) to about 1.000% (w/w), about 0.075% (w/w) to about 0.750% (w/w), about 0.075% (w/w) to about 0.500% (w/w), about 0.075% (w/w) to about 0.250% (w/w), about 0.075% (w/w) to about 0.200% (w/w), or about 0.075% (w/w) to about 0.150% (w/w).

In embodiments, a pharmaceutical composition is formulated for ophthalmic use.

In embodiments, a pharmaceutical composition is formulated for topical administration.

In embodiments, a pharmaceutical composition is formulated for administration using an injectable implant.

In embodiments, an injectable implant comprises a hydrogel.

In embodiments, a hydrogel comprises one or more cellulosic polymers.

In embodiments, a hydrogel comprises HPMC, HPC, polycarbophil, or xanthan gum.

In embodiments, an injectable implant comprises a biodegradable polymer.

In embodiments, an injectable implant comprises a polyester biodegradable polymer.

In embodiments, an injectable implant comprises a polyester biodegradable polymer that is PLA or PLGA.

In embodiments, an injectable implant releases the pharmaceutical composition for a period that is about 3-24 months, about 3-18 months, about 3-15 months, about 3-12 months, about 3-6 months, about 4-15 months, about 4-12 months, about 5-15 months, about 5-12 months, about 6-15 months, or about 6-12 months.

In embodiments, a pharmaceutical composition comprises a pharmaceutically acceptable excipient selected from tonicity agents, viscosity enhancing agents, buffering agents, pH adjusting agents, surfactants, preservatives, chelating agents, and combinations thereof.

In embodiments, a pharmaceutical composition has a pH that is about 6.5 to about 7.5, about 6.5 to about 7.0, about 7.0 to about 7.5, about 6.45 to about 6.55, about 6.55 to about 6.65, about 6.65 to about 6.75, about 6.75 to about 6.85, about 6.85 to about 6.95, about 6.95 to about 7.05, about 7.05 to about 7.15, about 7.15 to about 7.25, about 7.25 to about 7.35, about 7.35 to about 7.45, or about 7.45 to about 7.55.

In another aspect, the invention features a method of treating an ophthalmic disease in a patient, said method comprising administering to said patient an effective amount of any pharmaceutical composition comprising any compound described herein (e.g., any compound of formula (I), (II), (III), or (IV) such as Compound (1)).

In another aspect, the invention features a method of lowering intraocular pressure in a patient in need thereof, said method comprising administering to said patient an effective amount of any pharmaceutical composition comprising any compound described herein (e.g., any compound of formula (I), (II), (III), or (IV) such as Compound (1)).

In another aspect, the invention features a method of treating glaucoma in a patient in need thereof, said method comprising administering to said patient an effective amount of any pharmaceutical composition comprising any compound described herein (e.g., any compound of formula (I), (II), (III), or (IV) such as Compound (1)).

In embodiments, administering occurs one, two, three, or four times daily.

In embodiments, administering occurs once daily.

### BRIEF DESCRIPTION OF THE DRAWINGS

FIG. 1 shows intraocular pressure (IOP) obtained with (1) vehicle; (2) SEQ ID NO:1; (3) latanoprost; or (4) latanoprost and SEQ ID NO:1, as measured at the following time points: pre-dose, 0.5 h, 1 h, 2 h, 3 h, 4 h, 5 h, 6 h, 8 h, 12 h, and 24 h.

FIG. 2 shows a statistical analysis that confirms a synergistic effect based an area under the curve (AUC) analysis of the combination of latanoprost and SEQ ID NO:1, which afforded the greatest decrease in IOP.

FIGS. 3A-3E show maximum IOP reduction and statistical analyses (ANOVA and Tukey's) based on the maximum IOP reduction observed using (1) vehicle; (2) SEQ ID NO:1; (3) latanoprost; or (4) latanoprost and SEQ ID NO:1, as measured at 0-4 hours (FIG. 3A), 1 hour (FIG. 3B), 2 hours (FIG. 3C), 3 hours (FIG. 3D), or 4 hours (FIG. 3E).

FIG. 4 shows the mean delta percent change in intraocular pressure for female dogs dosed via topical ocular administration with 0.03% SEQ ID NO:1 in combination with increasing dose levels of latanoprost.

FIG. 5 shows the mean delta percent change in intraocular pressure for female dogs dosed via topical ocular administration with 0.005% latanoprost and/or 0.03% SEQ ID NO:1.

FIG. 6 shows the mean delta percent change in intraocular pressure for female dogs dosed via topical ocular administration with 0.005% latanoprost; 0.03% SEQ ID NO:1; and a combination of SEQ ID NO:1 and latanoprost.

FIG. 7 shows the intraocular pressure (mm Hg) for female dogs dosed via topical ocular administration with 0.005% latanoprost; 0.03% SEQ ID NO:1; and a combination of SEQ ID NO:1 and latanoprost.

FIG. 8 shows the mean delta percent change in intraocular pressure for female rabbits dosed via topical ocular administration with adjunct SEQ ID NO:1 0.1%/latanoprost 0.005%; and fixed-dose combination SEQ ID NO:1 0.1%/latanoprost 0.005%.

FIG. 9 shows the mean delta percent change in intraocular pressure for female dogs dosed via topical ocular administration with adjunct SEQ ID NO:1 0.1%/latanoprost 0.005%; and fixed-dose combination SEQ ID NO:1 0.1%/latanoprost 0.005%.

FIG. 10 shows the mean delta percent change in intraocular pressure for female dogs dosed via topical ocular administration with adjunct SEQ ID NO:1 0.1%/latanoprost 0.005%; fixed-dose combination SEQ ID NO:1 0.1%/latanoprost 0.005%; and fixed-dose combination SEQ ID NO:1 0.6%/latanoprost 0.005%.

FIG. 11 depicts studies of IOP reduction in dogs using travaprost (Travatan), timolol, and SEQ ID NO:1 in both monotherapy and in combination therapy. In combination therapies, travatan or timolol is dosed about 5 minutes before SEQ ID NO:1.

FIG. 12 depicts the significant IOP reduction in dogs using combination therapy of travaprost and SEQ ID NO:1.

FIG. 13 shows that the combination of travaprost and SEQ ID NO:1 results in a significant effect on the AUC in which prolonged duration of action is observed compared to monotherapy with SEQ ID NO:1. From left to right, the figure provides AUC values observed with: 0.03% SEQ ID NO:1; 0.004% Travatan; combination therapy with 0.03% SEQ ID NO:1 and 0.004% Travatan (Travatan dosed about five minutes before SEQ ID NO:1); 0.5% timolol; and combination therapy with 0.03% SEQ ID NO:1 and 0.5% timolol (timolol dosed about five minutes before SEQ ID NO:1).

### DETAILED DESCRIPTION OF THE INVENTION

### Definitions

Unless expressly stated otherwise or otherwise clear from context, the term “including” and its various forms (“include”, “includes”, etc.) is intended to be open-ended. That is, “including” means “including but not limited to”. The same applies to other terms of illustration, e.g., “such as”, “for example”, etc.

The term “alkyl” or “alkyl group,” as used herein, means a straight-chain (i.e., unbranched) or branched, substituted or unsubstituted hydrocarbon chain that is completely saturated. Unless otherwise specified, alkyl groups contain 1-8 carbon atoms. In some embodiments, alkyl groups contain 1-6 carbon atoms, and in some embodiments, alkyl groups contain 1-4 carbon atoms (represented as “C₁₋₄ alkyl”). In some embodiments, alkyl groups are characterized as “C₀₋₄ alkyl” representing either a covalent bond or a C₁₋₄ alkyl chain. Examples of alkyl groups include methyl, ethyl, propyl, butyl, isopropyl, isobutyl, sec-butyl, and tert-butyl.

The term “alkylene,” as used herein, represents a divalent form of an alkyl group as described herein and is exemplified by methylene, ethylene, isopropylene and the like.

The term “aralkyl” is used to describe a group wherein the alkyl chain can be branched or straight chain forming a linking portion with the terminal aryl, as defined herein, of the aryl-alkyl moiety. Examples of aralkyl groups include optionally substituted benzyl, phenethyl, phenpropyl and phenbutyl such as 4-chlorobenzyl, 2,4-dibromobenzyl, 2-methylbenzyl, 2-(3-fluorophenyl)ethyl, 2-(4-methylphenyl)ethyl, 2-(4-(trifluoromethyl)phenyl)ethyl, 2-(2-methoxyphenyl)ethyl, 2-(3-nitropheny ethyl, 2-(2,4-dichlorophenyl)ethyl, 2-(3,5-dimethoxyphenyl)ethyl, 3-phenylpropyl, 3-(3-chlorophenyl)propyli 3-(2-methylphenyl)propyl, 3-(4-methoxyphenyl)propyl, 3-(4-(trifluoromethyl)phenyl)propyl, 3-(2,4-dichlorophenyl)propyl, 4-phenylbutyl, 4-(4-chlorophenyl)butyl, 4-(2-methylphenyl)butyl, 4-(2,4-dichlorophenyl)butyl, 4-(2-methoxphenyl)butyl, and 10-phenyldecyl. Either portion of the moiety is unsubstituted or substituted.

The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 4 to 7 ring members. Examples of aryls include phenyl, naphthyl, and anthracene.

The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl.

The term “heteroalkylene,” as used herein, represents a divalent form of a heteroalkyl group as described herein.

As used herein, the term “heteroaryl”, used alone or as part of a larger moiety, refers to a monocyclic, bicyclic, or tricyclic aromatic ring system having a total of five to fourteen ring members, where at least one ring in the system is aromatic and in which at least one of the ring atoms is a heteroatom such as 0, N or S. Examples include 5-membered monocyclic rings such as thienyl, pyrrolyl, imidazolyl, pyrazolyl, furyl, isothiazolyl, furazanyl, isoxazolyl, thiazolyl and the like; and 6-membered monocyclic rings such as pyridyl, pyrazinyl, pyrimidinyl, pyridazinyl, triazinyl and the like. Further specific examples of heteroaryl rings include 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl and carbazolyl.

Unless otherwise specified, a molecular group described herein may be optionally substituted. As used herein, the phrase “optionally substituted” shall be understood by the skilled artisan to mean that the moiety to which the phrase refers may be unsubstituted, or it may be substituted with one or more (e.g., 1, 2, 3, 4, 5, or 6) substituent groups. Exemplary substituent groups include: halogen, oxo (═O), —CN, —OH, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl).

The term “about” or “approximately” means within an acceptable error range for the particular value as determined by one of ordinary skill in the art, which will depend in part on how the value is measured or determined, i.e., the limitations of the measurement system. In embodiments, “about” can mean within one standard deviation per the practice in the art. In embodiments, “about” refers to a range of up to 20%, up to 10%, up to 5%, or up to 1% of a given variable or value “x.” In embodiments, “about” can mean within one standard deviation per the practice in the art. In embodiments, “about” refers to a range of up to 10% of a given variable or value “x.”

The term “aqueous” typically denotes an aqueous composition (e.g., an aqueous pharmaceutical composition) wherein the carrier is to an extent of >50%, more preferably >75% and in particular >90% by weight water. These drops may be delivered from a single dose ampoule which may preferably be sterile and thus rendering bacteriostatic or bacteriocidal components of the formulation unnecessary. Alternatively, the drops may be delivered from a multi-dose bottle which may preferably comprise a device which extracts preservative from the formulation as it is delivered, such devices being known in the art.

The term “contemporaneous” as applied to administering more than one therapeutic agent (e.g., a linear peptide NPR-B agonist and a prostaglandin agonist as described herein) encompasses administering that is simultaneous and administering that is sequential.

The term “dose” as used herein refers to the amount of a particular pharmaceutically active ingredient used in a single administration event.

The terms “co-administration” and “administered in combination with,” encompass administration of two or more therapeutic agents to a subject so that both therapeutic agents, and/or metabolites thereof, are present in the subject at the same time. Co-administration includes simultaneous administration in separate compositions, administration at different times in separate compositions, or administration in a single composition in which both agents are present. Accordingly, in embodiments, co-administered agents may be in the same formulation. In other embodiments, co-administered agents may be in separate formulations.

The term “effective amount” or “therapeutically effective amount” refers to that amount of a compound or combination of compounds as described herein that is sufficient to effect the intended application (including disease treatment) as described herein. A therapeutically effective amount may vary depending upon the intended application (in vitro or in vivo), or the subject and disease condition being treated (e.g., the weight and age of the subject, the severity of the disease condition, the manner of administration and the like), which can readily be determined by one of ordinary skill in the art. The specific dose will vary depending on the particular compounds chosen, the dosing regimen to be followed, whether it is administered in combination with other compounds, timing of administration, the tissue to which it is administered, and the physical delivery system in which it is carried.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient.

The term “sequentially” as applied to administering more than one therapeutic agent (e.g., a first therapeutic agent that is a linear peptide NPR-B agonist and second therapeutic agent that is a prostaglandin agonist) at two different time points. In embodiments, a first therapeutic agent may be administered before a second therapeutic agent, where the times of administration are separated by about 2 hours, about 3 hours, about 4 hours, about 5 hours, about 6 hours, about 8 hours, or about 12 hours. In embodiments, a first therapeutic agent may be administered after a second therapeutic agent, where the times of administration are separated by about 2 hours, about 3 hours, about 4 hours, about 5 hours, about 6 hours, about 8 hours, or about 12 hours.

The term “simultaneous” or “simultaneously” as applied to administering more than one therapeutic agent (e.g., a linear peptide NPR-B agonist and a prostaglandin agonist as described herein) refers to administering the more than one therapeutic agent at the same time. In embodiments, simultaneous administration of two different therapeutic agents is effected by administering the more than one therapeutic agent in a single formulation. In embodiments, administration of two different therapeutic agents at the same time refers to the administration of each therapeutic agent at two different time points that are separated by no more than about 1 minute, about 2 minutes, about 5 minutes, about 10 minutes, about 15 minutes, about 30 minutes, about 45 minutes, or about 1 hour. In embodiments, administration of a first therapeutic agent occurs before or after administration of a second therapeutic agent, where the two administration events are separated by no more than about 1 minute, about 2 minutes, about 5 minutes, about 10 minutes, about 15 minutes, about 30 minutes, about 45 minutes, or about 1 hour.

A “sub-therapeutic amount” of an agent or therapy is an amount less than the effective amount for that agent or therapy, but when combined with an effective or sub-therapeutic amount of another agent or therapy can produce a therapeutic effect, due to, for example, synergy in the resulting efficacious effects, or reduced side effects.

The terms “synergistic” and “synergistically” as applied to the effect of two or more pharmaceutically active ingredients used in combination (whether simultaneously or sequentially) refer to a greater effect than when either of the two agents are used alone.

A “therapeutic effect,” as used herein, encompasses a therapeutic benefit and/or a prophylactic benefit as described herein (e.g., a reduction in IOP). A prophylactic benefit includes delaying or eliminating the appearance of a disease or condition, delaying or eliminating the onset of symptoms of a disease or condition, slowing, halting, or reversing the progression of a disease or condition, or any combination thereof.

A “disease” or “health-related condition” can be any pathological condition of a body part, an organ, or a system resulting from any cause, such as infection, trauma, genetic defect, age-related deterioration of bodily functions, and/or environmental stress. The cause may or may not be known. Examples of diseases include glaucoma, retinopathies, ocular trauma, and optic neuropathies. Thus, one of skill in the art realizes that a treatment may improve the disease condition, but may not be a complete cure for the disease.

The terms “prevention” and “preventing” are used herein according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking or minimizing the onset of a disease or health-related condition. For example, an individual with an eye that is at risk of developing glaucoma (such as an individual with ocular hypertension) can be treated with a compound or composition (e.g., a pharmaceutical composition) as described herein for the purpose of blocking or minimizing the onset of the signs or symptoms of glaucoma (e.g., prevention of glaucoma). In embodiments, prevention pertains to lowering elevated intraocular pressure, blocking detectable optic nerve damage as a result of glaucoma in a subject, reducing the rate of vision loss in a subject, or halting loss of vision in a subject. A subject can be a subject who is known or suspected of being free of a particular disease or health-related condition at the time the relevant preventive agent is administered. A subject, for example, can be a subject with no known disease or health-related condition (i.e., a healthy subject). In embodiments, the subject had a previous disease that has been treated in the past and is now known or suspected to be disease-free. Those skilled in the art understand that different diseases are summarized under certain terms or generic terms. These summaries are no limitation and each disease can be viewed on its own and can be treated or prevented with the compounds according to the present invention.

As used herein, the terms “susceptible,” or “susceptibility” refers to an individual or subject that is or at risk of developing optic nerve damage or retinal damage that is associated with elevated intraocular pressure.

“Treatment” and “treating” refer to administration or application of a compound or composition (e.g., a pharmaceutical composition) as described herein to a subject for the purpose of obtaining a therapeutic benefit of a disease or health-related condition. The term “therapeutic benefit” used throughout this application refers to anything that promotes or enhances the well-being of the subject with respect to the medical treatment of his condition. This includes a reduction in the frequency or severity of the signs or symptoms of a disease. Therapeutic benefit also includes reducing the signs or symptoms associated with glaucoma in a subject with glaucoma. For example, a therapeutic benefit in a patient with glaucoma is obtained where there is no further progression of visual field loss in the affected eye, or a slowing of the rate of progression of visual field loss in the affected eye, or an improvement in vision.

“Glaucoma” and “glaucomatous optic neuropathy” and “glaucomatous retinopathy,” as used herein, are interchangeable. Glaucoma refers to a disease characterized by the permanent loss of visual function due to irreversible damage to the retinal ganglion cells in the retina and optic nerve. The major risk factor for glaucoma and the related loss of visual function is elevated intraocular pressure. There are different types of glaucoma, including primary open angle glaucoma (POAG), angle closure glaucoma, and congenital/developmental glaucoma.

As used herein, the term “intraocular pressure” or “IOP” refers to the pressure of the content inside the eye. In a normal human eye, IOP is typically in the range of 10 to 21 mm Hg. IOP varies among individuals, for example, it may become elevated due to anatomical problems, inflammation of the eye, as a side-effect from medication or due to genetic factors. “Elevated” intraocular pressure is currently considered to be 21 mm Hg, which is also considered to be a major risk factor for the development of glaucoma.

“Ocular hypertension” as used herein refers to a condition in which the intraocular pressure in the eye of a subject is higher than normal but the optic nerve and visual fields are within normal limits. These individuals may be susceptible to developing the loss of visual function that is typically associated with glaucoma.

### Conjugates and Compositions Thereof

In embodiments, the invention features a compound comprising a first substructure comprising a linear peptidic NPR-B agonist; where said first substructure comprises a covalent attachment (e.g., via a covalent bond or another linker group as described herein) to a second substructure comprising a prostaglandin agonist (e.g., a second substructure formed from a prostaglandin agonist).

In embodiments, a first substructure comprises (e.g., is formed from) a linear peptidic NPR-B agonist that is a peptide as described in, e.g., U.S. Pat. No. 8,546,523 or International Publication No. WO 2011/038061, each of which is hereby incorporated by reference in its entirety.

In embodiments, a first substructure comprises (e.g., is formed from) a linear peptidic NPR-B agonist that is a compound according to the following formula (A),

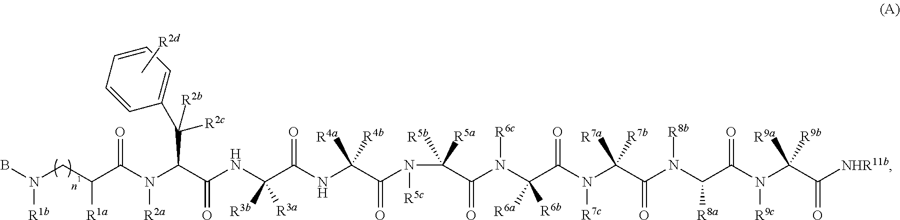

wherein

one (e.g., one and only one) of B, R^{b1}, R^{1a}, R^{2a}, R^{2b}, R^{2c}, R^{2d}, R^{3a}, R^{3b}, R^{4a}, R^{4b}, R^{5a}, R^{5b}, R^{5c}, R^{6a}, R^{6b}, R^{6c}, R^{7a}, R^{7b}, R^{7c}, R^{8a}, R^{8b}, R^{9a}, R^{9b}, R^{9c}, and R^{11b}, or exemplary combinations of these variables as described herein, comprises a covalent attachment (e.g., via a covalent bond or via a linker as described herein) or is a covalent attachment to said second substructure, and wherein still other exemplary permitted groups for these moieties are as follows:

B is selected from the group consisting of R^{b1}— and R^{b2}—C(O)—, wherein:
R^{b1} is selected from the group consisting of C₆-C₁₀ alkyl and C₆-C₁₀ alkyl substituted by NR^{b4}R^{b5};
R^{b2} is selected from the group consisting of C₆-C₁₀ alkyl and C₆-C₁₀ alkyl substituted by NR^{b4}R^{b5}; and
R^{b4} and R^{b5} are, independently, selected from the group consisting of H and C₁-C₄ alkyl;

R^{1a} is selected from H and C₁-C₄ alkyl; and

R^{1b} is selected from H, C₁-C₄ alkyl optionally substituted by OH, and hydroxy C₁-C₄ alkyl optionally substituted by OH; or alternatively,

R^{1a} and R^{1b} together form a heterocyclic ring;

n¹ is 0 or 1;

R^{2a} is selected from the group consisting of H, methyl, ethyl, propyl, isopropyl, C₁-C₂ alkyl C₃-C₇ cycloalkyl and aryl C₁-C₂ alkyl;

R^{2b} and R^{2c} are, independently, selected from the group consisting of H, methyl, ethyl, propyl, and isopropyl, with the proviso that at least one of R^{2b} and R^{2c} is H;

R^{2d} represents from 0 to 3 substituents, each such substituent being, independently, selected from the group consisting of H, Cl, F, Br, CN, CF₃, OH, OR^{2e} and C₁-C₄ alkyl, wherein R^{2e} is selected from the group consisting of methyl, ethyl, propyl, and isopropyl;

R^{3a} is selected from the group consisting of H and C₁-C₄alkyl; and

R^{3b} is selected from the group consisting of H, and —(CH₂)ₙ₃ₐ—X^{3a}, wherein
n3a is 1 to 5; and
X^{3a} is selected from the group consisting of H and NR^{3c}R^{3d}, wherein R^{3c} and

R^{3d} are independently selected from the group consisting of H, C₁-C₈ alkyl, and —(C═N)—NH₂; or alternatively,

R^{3a} and R^{3b} are linked to form a cyclic structure; or alternatively,

R^{3a} and R^{3b} are linked with a heteroatom selected from the group consisting of N, O, and S, to form a heterocyclic structure;

R^{4a} is selected from the group consisting of H, and C₁-C₈alkyl which is optionally substituted with a moiety selected from the group consisting of OH, and CO₂R^{4c}, wherein R^{4c} is selected from the group consisting of H, and C₁-C₃alkyl;

R^{4b} is selected from the group consisting of H and methyl;

R^{5a} is (CH₂)ₙ₅ₐ—X^{5a}, wherein
n5a is 1 to 6; and
X^{5a} is selected from the group consisting of H, NH₂, and a C₄-C₇ amine-containing aliphatic heterocyclic ring; and

R^{5c} is selected from the group consisting of H and methyl; or alternatively,

R^{5c} and R^{5a} combine to form a4 to 6 membered heterocyclic ring, wherein said heterocyclic ring has from 0 to 2 substituents, each such substituent independently selected from the group consisting of OH, F, C₁-C₄ alkyl, —NHC(═NH)NH₂, aryl, and NR^{5e}R^{5f}, wherein
R^{5e} is selected from the group consisting of H, C₁-C₄ alkyl, —C(═O)(CH₂)_{n5b}— X^{5b}, and —CH₂(CH₂)_{n5c}—X^{5b}; and
R^{5f} is selected from the group consisting of H, C₁-C₄ alkyl, and —CH₂(CH₂)_{n5d}—X^{5c}; wherein
   n5b is selected from the group consisting of 1, 2, 3, and 4;
   n5c and n5d are independently selected from the group consisting of 2, 3, and 4; and
   X^{5b} and X^{5c} are independently selected from the group consisting of H and NR^{5g}R^{5h}; wherein R^{5g} and R^{5h} are independently selected from the group consisting of H and C₁-C₄ alkyl;

R^{5b} is selected from the group consisting of H and methyl;

R^{6a} is selected from the group consisting of C₁-C₈alkyl, aryl C₁-C₄alkyl, C₄-C₂cycloalkyl C₁-C₄alkyl, and C₄-C₂cycloalkyl, wherein each of said C₁-C₈alkyl and C₄-C₂cycloalkyl is optionally substituted with a moiety selected from the group consisting of OH, and O(C₁-C₄alkyl);

R^{6b} is H;

R^{6c} is selected from the group consisting of H and C₁-C₄alkyl;

R^{7a} is selected from the group consisting of C₁-C₄alkyl, C₃-C₂cycloalkyl, 2-thienyl, (CH₂)ₙ₇ₐ—X^{7a}, and C₁-C₄alkyl substituted with OH, wherein
n^{7a} is selected from the group consisting of 1 and 2; and
X^{7a} is selected from the group consisting of 2-thienyl, C(═O)OR^{7e}, C(═O)NH₂,

S(═O)₂OH, OS(═O)₂OH, B(OH)₂, P(═O)(OH)₂, and OP(═O)(OH)₂, wherein R^{7e} is selected from the group consisting of H and C₁-C₄alkyl;

R^{7b} is H or 2-thienyl;

R^{7c} is selected from the group consisting of H and methyl;

R^{8a} is (CH₂)ₘ₈ₐ—X^{8a}, wherein
m^{8a}=1-5;
X^{8a} is selected from the group consisting of H, NH₂, and —NHC(═NH)NH₂;

R^{8b} is selected from the group consisting of H and methyl;

R^{9a} is selected from the group consisting of C₁-C₅alkyl and C₄-C₂cycloalkyl; and

R^{9b} is selected from the group consisting of H and C₁-C₅alkyl; or alternatively,

R^{9a} and R^{9b} form a 5-7 membered cycloalkyl ring;

R^{9c} is selected from the group consisting of H and methyl; and

R^{11b} is selected from the group consisting of H, C₁-C₈alkyl, C₄-C₈ cycloalkyl, C₂-C₁₂bicycloalkyl, C₇-C₁₂ cycloalkylaryl, and C₁-C₄alkyl-C₄-C₈cycloalkyl.

In embodiments, the optionally substituted moiety formed by the combination of R^{5c} and R^{5a} to form a 4 to 6 membered heterocyclic ring, comprises a covalent attachment (e.g., via a covalent bond or via a linker as described herein) to said second substructure, and permitted groups for moieties B, R^{b1}, R^{1a}, R^{2a}, R^{2b}, R^{2c}, R^{2d}, R^{3a}, R^{3b}, R^{4a}, R^{4b}, R^{5b}, R^{6a}, R^{6b}, R^{6c}, R^{7a}, R^{7b}, R^{7c}, R^{8a}, R^{8b}, R^{9a}, R^{9b}, R^{9c}, and R^{11b} are as described herein. In embodiments, said covalent attachment is via modification of a hydroxyl substituent on said 4 to 6 membered heterocyclic ring.

In embodiments, R^{7a} comprises a covalent attachment (e.g., via a covalent bond or via a linker as described herein) to said second substructure, and permitted groups for moieties B, R^{b1}, R^{1a}, R^{2a}, R^{2b}, R^{2c}, R^{2d}, R^{3a}, R^{3b}, R^{4a}, R^{4b}, R^{5b}, R^{6a}, R^{6b}, R^{6c}, R^{7a}, R^{7b}, R^{7c}, R^{8a}, R^{8b}, R^{9a}, R^{9b}, R^{9c}, and R^{11b} are as described herein. In embodiments, R₇ₐ is —(CH₂)ₙ₇ₐ—C(═O)—O-L or R₇ₐ is —(CH₂)ₙ₇ₐ—C(═O)—NH-L, wherein L represents said covalent attachment (e.g., via a covalent bond or via a linker as described herein) to said second substructure.

In embodiments, a first substructure comprises (e.g., is formed from) a linear peptidic NPR-B agonist that is a compound of formula (B-1),

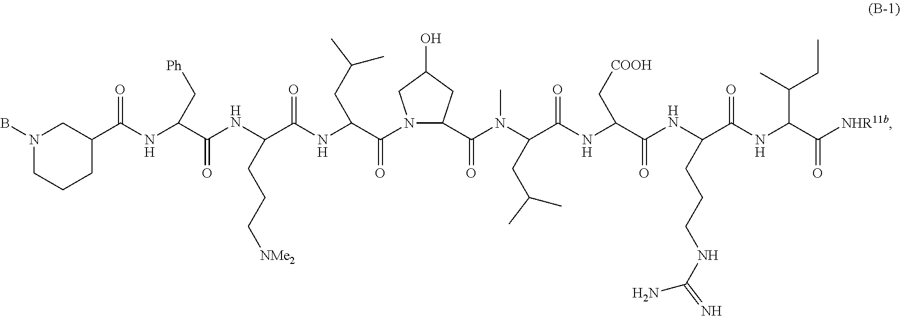

or a pharmaceutically acceptable salt thereof, wherein

B is selected from the group consisting of R^{b1}— and R^{b2}—C(O)—;

R^{b1} is selected from the group consisting of C₆-C₁₀ alkyl and C₆-C₁₀ alkyl substituted by NR^{b4}R^{b5};

R^{b2} is selected from the group consisting of C₆-C₁₀ alkyl and C₆-C₁₀ alkyl substituted by NR^{b4}R^{b5}; R^{b4} and R^{b5} are, independently, selected from the group consisting of H and C₁-C₄ alkyl; and

R^{11b} is selected from the group consisting of H, C₁-C₈ alkyl, C₄-C₈ cycloalkyl, C₇-C₁₂ bicycloalkyl, and C₁-C₄ alkyl-C₄-C₈ cycloalkyl.

In embodiments, the compound of formula (B-1) is a compound of formula (B-2),

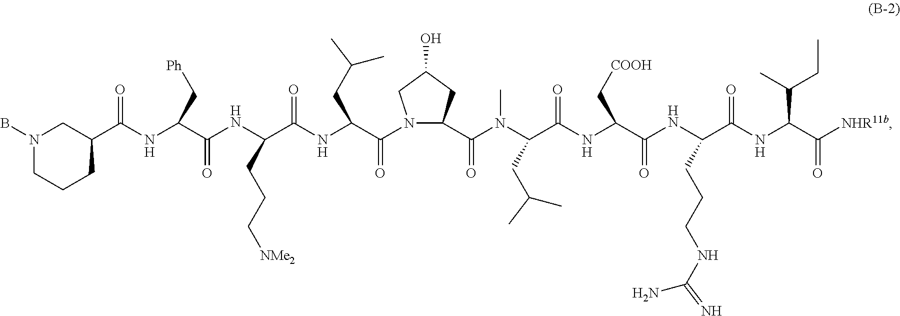

wherein one of the residues is modified to comprise a covalent attachment (e.g., via a covalent bond or via a linker as described herein) to said second substructure.

B is selected from the group consisting of R^{b1}— and R^{b2}—C(O)—.

R^{b1} is selected from the group consisting of C₆-C₁₀ alkyl and C₆-C₁₀ alkyl substituted by NR^{b4}R^{b5}.

R^{b2} is selected from the group consisting of C₆-C₁₀ alkyl and C₆-C₁₀ alkyl substituted by NR^{b4}R^{b5}.

R^{b4} and R^{b5} are, independently, selected from the group consisting of H and C₁-C₄ alkyl.

R^{11b} is selected from the group consisting of H, C₁-C₈ alkyl, C₄-C₈ cycloalkyl, C₂-C₁₂ bicycloalkyl, and C₁-C₄ alkyl-C₄-C₈ cycloalkyl.

In embodiments, B is R^{b2}—C(O)— and R^{b2} is C₆-C₁₀ alkyl; and R^{11b} is selected from the group consisting of H and C₁-C₈ alkyl.

In embodiments, residue “Hyp” (a residue formed form (2S,4R)-4-hydroxypyrrolidine-2-carboxylic acid) is modified to comprise a covalent attachment (e.g., a covalent bond or another linker group as described herein) to said second substructure. In embodiments, residue “Hyp” comprises a covalent attachment (e.g., a covalent bond or another linker group as described herein) to said second substructure via modification of a hydroxyl (—OH) moiety.

In embodiments, residue “Asp” (a residue formed from aspartic acid) is modified to comprise a covalent attachment (e.g., a covalent bond or another linker group as described herein) to said second substructure. In embodiments, residue “Asp” comprises a covalent attachment (e.g., a covalent bond or another linker group as described herein) to said second substructure via modification of a carboxylic acid (—CO₂H) moiety.

In embodiments, a first substructure comprises (e.g., is formed from) a linear peptidic NPR-B agonist that has the following sequence,
Occ-Sni-Phe-orn(Me2)-Leu-Hyp-Nml-Asp-Arg-Ile-NH₂ (SEQ ID NO:1);
   wherein one of the residues is modified to comprise a covalent attachment to said second substructure (e.g., a derivative of an amino acid residue that has been modified to comprise a covalent bond or another linker group as described herein to said second substructure) and wherein
“Occ” represents a moiety formed from octanoic acid or a derivative thereof;
“Sni” represents a residue formed from (S)-nipecotic acid or a derivative thereof;
“orn(Me2)” represents a residue formed form (R)-2-amino-5-(dimethylamino) pentanoic acid or a derivative thereof;
“Leu” is a standard abbreviation and represents a residue formed from leucine or a derivative thereof;
“Hyp” represents a residue formed form (2S,4R)-4-hydroxypyrrolidine-2-carboxylic acid or a derivative thereof;
“Nml” represents a residue formed from (S)—N-methyl-leucine or a derivative thereof;
“Asp” is a standard abbreviation and represents a residue formed from aspartic acid or a derivative thereof;
“Arg” is a standard abbreviation and represents a residue formed from arginine or a derivative thereof; and
“Ile” is a standard abbreviation and represents a residue formed from isoleucine or a derivative thereof.

In embodiments, residue “Hyp” comprises a covalent attachment (e.g., a covalent bond or another linker group as described herein) to said second substructure.

In embodiments, residue “Asp” comprises a covalent attachment (e.g., a covalent bond or another linker group as described herein) to said second substructure.

In embodiments, said second substructure comprises a prostaglandin agonist.

In embodiments, said second substructure comprises a prostaglandin agonist that reduces intraocular pressure (IOP) in a subject in need thereof (e.g., said second substructure is formed from a prostaglandin agonist that reduces intraocular pressure (IOP) in a subject in need thereof), wherein said second substructure comprises a covalent attachment (e.g., a covalent bond or another linker group as described herein) to said first substructure.

In embodiments, said second substructure comprises a prostaglandin agonist suitable for treating or preventing glaucoma in a patient in need thereof (e.g., said second substructure is formed form a prostaglandin agonist suitable for treating or preventing glaucoma in a patient in need thereof), wherein said second substructure comprises a covalent attachment (e.g., a covalent bond or another linker group as described herein) to said first substructure.

In embodiments, said second substructure comprises a prostaglandin agonist that is Xalatan® (latanoprost), Lumigan® (bimatoprost), Travatan Z® (travoprost), or Zioptan™ (tafluprost), wherein said prostaglandin agonist comprises a covalent attachment (e.g., via a covalent bond or another linker group as described herein) to said first substructure. In embodiments, said second substructure is formed from a prostaglandin agonist that is Xalatan® (latanoprost), Lumigan® (bimatoprost), Travatan Z® (travoprost), or Zioptan™ (tafluprost), wherein said prostaglandin agonist comprises a covalent attachment (e.g., via a covalent bond or another linker group as described herein) to said first substructure.

In embodiments, said second substructure is formed from the following compound,

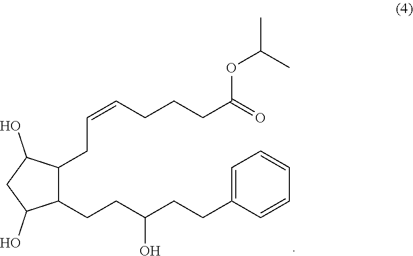 [SMILES: CC(C)OC(=O)CCC/C=C\CC1C(O)CC(O)C1CCC(O)CCc1ccccc1]

In embodiments, said second substructure is formed from latanoprost,

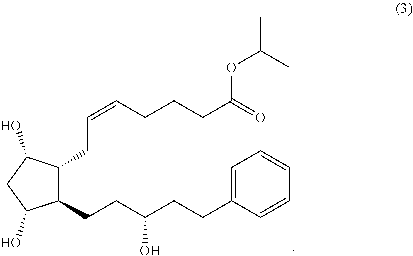 [SMILES: CC(C)OC(=O)CCC/C=C\C[C@@H]1[C@@H](CC[C@@H](O)CCc2ccccc2)[C@H](O)C[C@@H]1O]

In embodiments, said second substructure comprises latanoprost (e.g., is formed from latanoprost). In embodiments, said second substructure comprises a covalent attachment to said first substructure via modification of the carboxylic ester (—CO₂ⁱPr) of latanoprost. In embodiments, said second substructure comprises a covalent attachment to said first substructure via modification of a hydroxyl (—OH) moiety of latanoprost.

In embodiments, said second substructure is formed from the following compound,

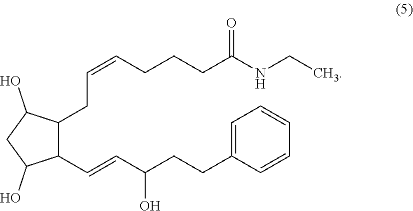 [SMILES: CCNC(=O)CCC/C=C\CC1C(O)CC(O)C1/C=C/C(O)CCc1ccccc1]

In embodiments, said second substructure is formed from bimatoprost,

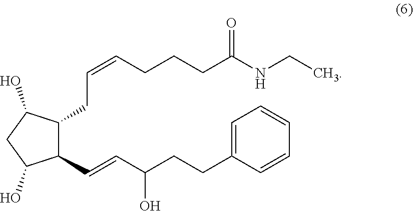 [SMILES: CCNC(=O)CCC/C=C\C[C@@H]1[C@@H](/C=C/C(O)CCc2ccccc2)[C@H](O)C[C@@H]1O]

In embodiments, said second substructure comprises bimatoprost. In embodiments, said second substructure comprises a covalent attachment to said first substructure via modification of the carboxylic amide (—CONHEt) of bimatoprost. In embodiments, said second substructure comprises a covalent attachment to said first substructure via modification of a hydroxyl (—OH) moiety of bimatoprost.

In embodiments, said second substructure is formed from the following compound,

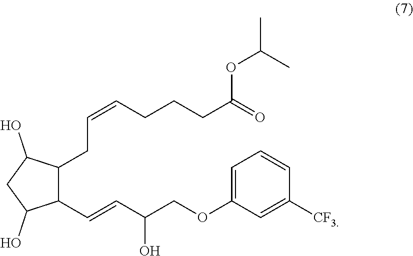 [SMILES: CC(C)OC(=O)CCC/C=C\CC1C(O)CC(O)C1/C=C/C(O)COc1cccc(C(F)(F)F)c1]

In embodiments, said second substructure is formed from travoprost,

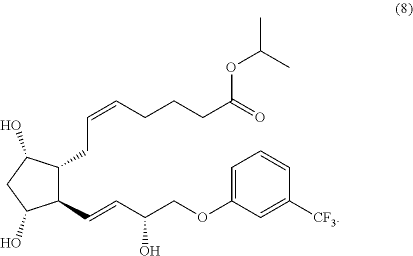 [SMILES: CC(C)OC(=O)CCC/C=C\C[C@@H]1[C@@H](/C=C/[C@@H](O)COc2cccc(C(F)(F)F)c2)[C@H](O)C[C@@H]1O]

In embodiments, said second substructure comprises travoprost. In embodiments, said second substructure comprises a covalent attachment to said first substructure via modification of the carboxylic ester (—CO₂ⁱPr) of travoprost. In embodiments, said second substructure comprises a covalent attachment to said first substructure via modification of a hydroxyl (—OH) moiety of travoprost.

In embodiments, said second substructure is formed from the following compound,

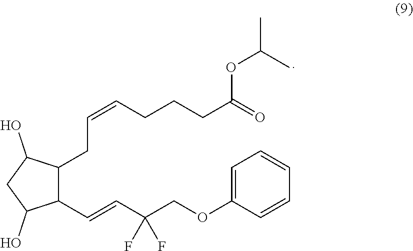 [SMILES: CC(C)OC(=O)CCC/C=C\CC1C(O)CC(O)C1/C=C/C(F)(F)COc1ccccc1]

In embodiments, said second substructure is formed from tafluprost,

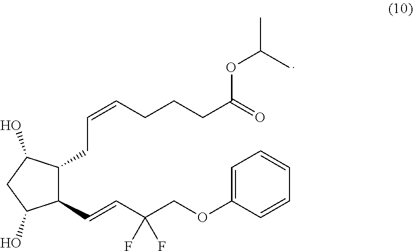 [SMILES: CC(C)OC(=O)CCC/C=C\C[C@@H]1[C@@H](/C=C/C(F)(F)COc2ccccc2)[C@H](O)C[C@@H]1O]

In embodiments, said second substructure comprises tafluprost. In embodiments, said second substructure comprises a covalent attachment to said first substructure via modification of the carboxylic ester (—CO₂ⁱPr) of tafluprost. In embodiments, said second substructure comprises a covalent attachment to said first substructure via modification of a hydroxyl (—OH) moiety of tafluprost.

In embodiments, a covalent attachment between said first substructure and said second substructure is represented as moiety L.

In embodiments, L is a covalent bond.

In embodiments, L is optionally substituted C₁-C₂₀ alkylene, optionally substituted C₂-C₂₀ alkenylene, or optionally substituted C₂-C₂₀ alkynylene. In embodiments, L is unsubstituted. In embodiments, L is substituted (e.g., with 1, 2, 3, 4, 5, or 6 substituent groups such as those described herein).

In embodiments, L is optionally substituted 2- to 20-membered heteroalkylene. In embodiments, L is optionally substituted 3- to 20-membered heteroalkenylene. In embodiments, L is optionally substituted 3- to 20-membered heteroalkynylene. In embodiments, L is unsubstituted. In embodiments, L is substituted (e.g., with 1, 2, 3, 4, 5, or 6 substituent groups such as those described herein).

In embodiments, L is a peptidic linker (e.g., L comprises amino acid residues).

In embodiments, L is a non-peptidic linker (e.g., L does not comprise any amino-acid residues).

In embodiments, the invention features a compound of formula (I),

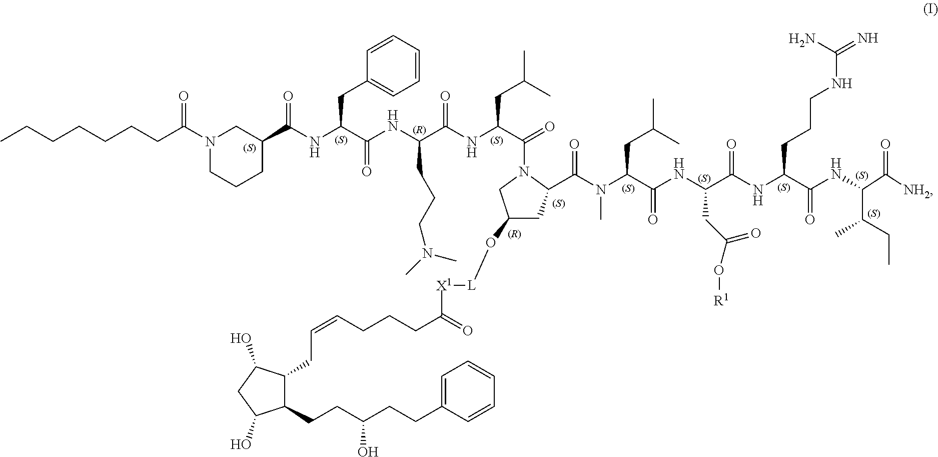

or a pharmaceutically acceptable salt thereof, wherein

X¹ is a covalent bond, —O—, —S—, or —NR^{X1}—;

R¹ is H, optionally substituted C₁-C₁₂ alkyl, or optionally substituted C₇-C₁₆ aralkyl;

R^{X1} is H or optionally substituted C₁-C₁₂ alkyl;

L represents a linker that is a covalent bond, optionally substituted C₁-C₁₂ alkylene, or optionally substituted 2- to 12-membered heteroalkylene, or

L represents a linker having the structure -(L¹)-L²-(L³)-;
each of L¹ and L³ is independently a covalent bond, optionally substituted C₁-C₆ alkylene, or optionally substituted 2- to 6-membered heteroalkylene; and
L² is optionally substituted C₆-C₁₀ arylene or optionally substituted 5- to 10-membered heteroarylene.

In embodiments, R¹ is H. In embodiments, R¹ is optionally substituted C₁-C₁₂ alkyl. In embodiments, R¹ is optionally substituted C₂-C₁₆ aralkyl.

In embodiments, R¹ is H, methyl, ethyl, isopropyl, n-propyl, tert-buyl, or benzyl.

In embodiments, R¹ is H.

In embodiments, X¹ is a covalent bond. In embodiments, X¹ is —O—. In embodiments, X¹ is —S—. In embodiments, X¹ is —NR^{X1}—.

In embodiments, X¹ is —O—.

In embodiments, R^{X1} is H. In embodiments, R^{X1} is optionally substituted C₁-C₁₂ alkyl.

In embodiments, L represents a linker that is a covalent bond. In embodiments, L represents a linker that is optionally substituted C₁-C₁₂ alkylene (e.g., optionally substituted C₂-C₁₂ alkylene). In embodiments, L represents a linker that is optionally substituted 2- to 12-membered heteroalkylene. In embodiments, L represents a linker having the structure -(L¹)-L²-(L³)-.

In embodiments, each of L¹ and L³ is independently a covalent bond, optionally substituted C₁-C₆ alkylene, or optionally substituted 2- to 6-membered heteroalkylene. In embodiments, each of L¹ and L³ is independently a covalent bond. In embodiments, one of L¹ and L³ is a covalent bond, and the other is optionally substituted C₁-C₆ alkylene, or optionally substituted 2- to 6-membered heteroalkylene. In embodiments, each of L¹ and L³ is independently optionally substituted C₁-C₆ alkylene or optionally substituted 2- to 6-membered heteroalkylene.

In embodiments, L² is optionally substituted C₆-C₁₀ arylene.

In embodiments, L² is optionally substituted 5- to 10-membered heteroarylene.

In embodiments, linker L is optionally substituted C₁-C₁₂ alkylene (e.g., optionally substituted C₂-C₁₂ alkylene). In embodiments, linker L is unsubstituted C₁-C₁₂ alkylene. In embodiments, linker L is substituted C₁-C₁₂ alkylene comprising 1, 2, 3, 4, 5, or 6 substituent groups such as the exemplary substituent groups described herein.

In embodiments, linker L is optionally substituted 2- to 12-membered heteroalkylene. In embodiments, linker L is unsubstituted 2- to 12-membered heteroalkylene. In embodiments, linker L is substituted 2- to 12-membered heteroalkylene comprising 1, 2, 3, 4, 5, or 6 substituent groups such as the exemplary substituent groups described herein.

In embodiments, linker L comprises one or more ethylene glycol substructures (e.g., 1, 2, 3, 4, 5, or 6 ethylene glycol substructures).

In embodiments, linker L comprises a substructure that is —CH₂CH₂—O—CH₂CH₂— or —CH₂CH₂—O—CH₂CH₂—O—CH₂CH₂—.

In embodiments, linker L comprises a substituent group that is an oxo (═O) moiety. In embodiments, linker L comprises no further substituents. In embodiments, Linker L comprises additional substituents (e.g., 1, 2, 3, 4, or 5 additional substituent groups such as the exemplary substituent groups described herein).

In embodiments, linker L is —(CH₂)ₙ—C(═O)—, and n is 2, 3, 4, or 5. In embodiments, n is 2. In embodiments, n is 3. In embodiments, n is 4. In embodiments, n is 5.

In embodiments, linker L is —CH₂CH₂—O—CH₂CH₂—C(═O)—, —CH₂CH₂—O—CH₂CH₂—O—CH₂CH₂—C(═O)—, —CH₂CH₂—O—CH₂CH₂—O—C(═O)—, or —CH₂CH₂—O—CH₂CH₂—O—CH₂CH₂—O—C(═O)—. In embodiments, linker L is —CH₂CH₂—O—CH₂CH₂—C(═O)—. In embodiments, linker L is —CH₂CH₂—O—CH₂CH₂—O—CH₂CH₂—C(═O)—. In embodiments, linker L is —CH₂CH₂—O—CH₂CH₂—O—C(═O)—. In embodiments, linker L is —CH₂CH₂—O—CH₂CH₂—O—CH₂CH₂—O—C(═O)—.

In embodiments, each of X¹ and L is a covalent bond.

In embodiments, the compound is a compound of formula (II),

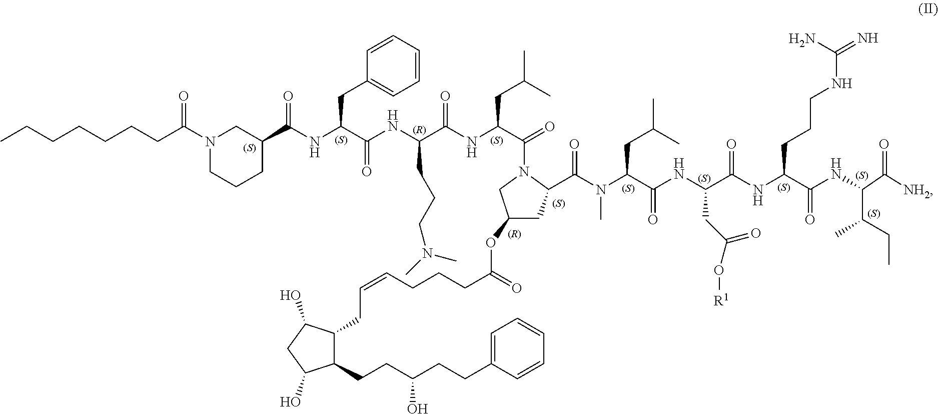

or a pharmaceutically acceptable salt thereof, wherein

R¹ is H, optionally substituted C₁-C₁₂ alkyl, or optionally substituted C₇-C₁₆ aralkyl.

In embodiments, R¹ is H. In embodiments, R¹ is optionally substituted C₁-C₁₂ alkyl. In embodiments, R¹ is optionally substituted C₇-C₁₆ aralkyl.

In embodiments, R¹ is H, methyl, ethyl, isopropyl, n-propyl, tert-buyl, or benzyl.

In embodiments, R¹ is H.

In embodiments, the compound is a compound of formula (III),

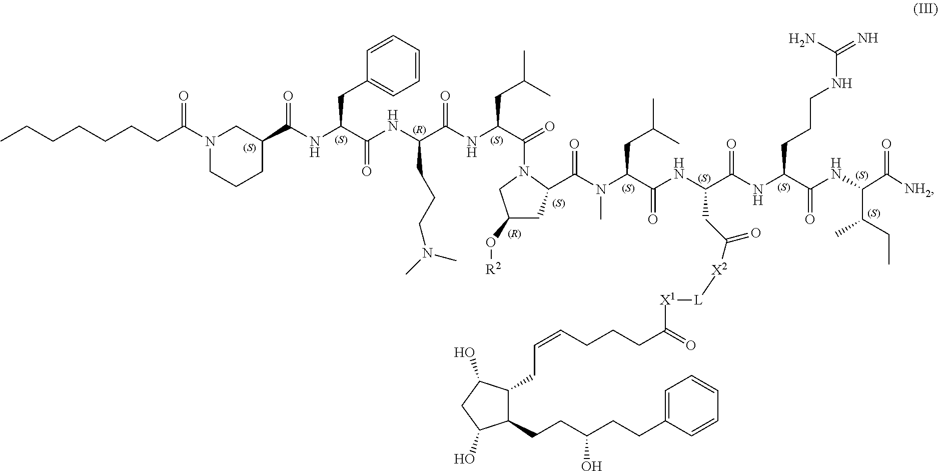

or a pharmaceutically acceptable salt thereof, wherein

R² is H, optionally substituted C₁-C₁₂ alkyl, or optionally substituted C₇-C₁₆ aralkyl;

X¹ is a covalent bond, —O—, —S—, or —NR^{x1}—;

X² is a covalent bond, —O—, —S—, or —NR^{X2}—;

each of R^{X1} and R^{X2} is independently H or optionally substituted C₁-C₁₂ alkyl;

L represents a linker that is a covalent bond, optionally substituted C₁-C₁₂ alkylene, or optionally substituted 2- to 12-membered heteroalkylene, or

L represents a linker having the structure -(L¹)-L²-(L³)-;
each of L¹ and L³ is independently a covalent bond, optionally substituted C₁-C₆ alkylene, or optionally substituted 2- to 6-membered heteroalkylene; and
L² is optionally substituted C₆-C₁₀ arylene or optionally substituted 5- to 10-membered heteroarylene.

In embodiments, R² is H. In embodiments, R² is optionally substituted C₁-C₁₂ alkyl. In embodiments, R² is optionally substituted C₇-C₁₆ aralkyl.

In embodiments, R² is H or R² is acetyl, propionyl, pivaloyl, octanoyl, cyclopropanolyl, cyclohexanoyl, or benzyl, each of which is optionally substituted.

In embodiments, R² is H.

In embodiments, X¹ is a covalent bond. In embodiments, X¹ is —O—. In embodiments, X¹ is —S—. In embodiments, X¹ is —NR^{X1}—. In embodiments, R^{X1} is H. In embodiments, R^{X1} is optionally substituted C₁-C₁₂ alkyl.

In embodiments, X² is a covalent bond. In embodiments, X² is —O—. In embodiments, X² is —S—. In embodiments, X² is —NR^{X2}—. In embodiments, R^{x2} is H. In embodiments, R^{x2} is optionally substituted C₁-C₁₂ alkyl.

In embodiments, X¹ is —O—.

In embodiments, X² is —O—.

In embodiments, L represents a linker that is a covalent bond.

In embodiments, L represents a linker that is optionally substituted C₁-C₁₂ alkylene (e.g., optionally substituted C₂-C₁₂ alkylene).

In embodiments, L represents a linker that is optionally substituted 2- to 12-membered heteroalkylene.

In embodiments, L represents a linker having the structure -(L¹)-L²-(L³)-.

In embodiments, L¹ is independently a covalent bond. In embodiments, L¹ is optionally substituted C₁-C₆ alkylene. In embodiments, L¹ is optionally substituted 2- to 6-membered heteroalkylene.

In embodiments, L³ is independently a covalent bond. In embodiments, L³ is optionally substituted C₁-C₆ alkylene. In embodiments, L³ is optionally substituted 2- to 6-membered heteroalkylene.

In embodiments, L² is optionally substituted C₆-C₁₀ arylene. In embodiments, L² is optionally substituted 5- to 10-membered heteroarylene.

In embodiments, linker L is optionally substituted C₁-C₁₂ alkylene (e.g., optionally substituted C₂-C₁₂ alkylene). In embodiments, linker L is unsubstituted C₁-C₁₂ alkylene. In embodiments, linker L is substituted C₁-C₁₂ alkylene (e.g., C₁-C₁₂ alkylene having 1, 2, 3, 4, 5, or 6 substituent groups such as those described herein).

In embodiments, linker L is optionally substituted 2- to 12-membered heteroalkylene. In embodiments, linker L is unsubstituted 2- to 12-membered heteroalkylene. In embodiments, linker L is substituted 2- to 12-membered heteroalkylene (e.g., 2- to 12-membered heteroalkylene having 1, 2, 3, 4, 5, or 6 substituent groups such as those described herein).

In embodiments, linker L comprises one or more ethylene glycol substructures (e.g., 1, 2, 3, 4, 5, or 6 ethylene glycol structures).

In embodiments, linker L comprises a substructure that is —CH₂CH₂—O—CH₂CH₂— or —CH₂CH₂—O—CH₂CH₂—O—CH₂CH₂—. In embodiments, linker L comprises a substructure that is —CH₂CH₂—O—CH₂CH₂—. In embodiments, linker L comprises a substructure that is —CH₂CH₂—O—CH₂CH₂—O—CH₂CH₂—.

In embodiments, linker L comprises a substituent group that is an oxo (═O) moiety. In embodiments, linker L comprises no further substituents. In embodiments, Linker L comprises additional substituents (e.g., 1, 2, 3, 4, or 5 additional substituent groups such as the exemplary substituent groups described herein).

In embodiments, linker L is —(CH₂)ₙ—C(═O)—, and n is 2, 3, 4, or 5. In embodiments, n is 2. In embodiments, n is 3. In embodiments, n is 4. In embodiments, n is 5.

In embodiments, linker L is —CH₂CH₂—O—CH₂CH₂—C(═O)— or —CH₂CH₂—O—CH₂CH₂—O—CH₂CH₂—C(═O)—. In embodiments, linker L is —CH₂CH₂—O—CH₂CH₂—C(═O)—. In embodiments, linker L is —CH₂CH₂—O—CH₂CH₂—O—CH₂CH₂—C(═O)—.

In embodiments, the compound is a compound of formula (IV):

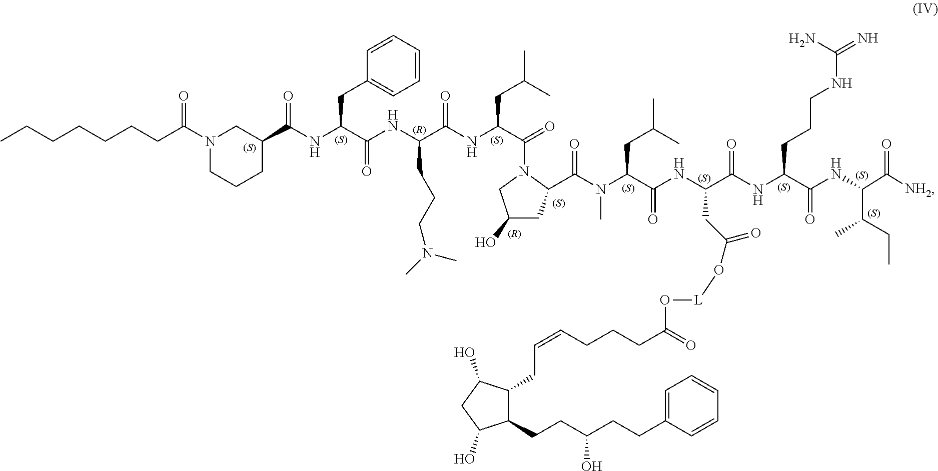

or a pharmaceutically acceptable salt thereof, wherein

L represents a linker that is optionally substituted C₁-C₁₂ alkylene or optionally substituted 2- to 12-membered heteroalkylene.

In embodiments, L is optionally substituted C₁-C₁₂ alkylene (e.g., optionally substituted C₂-C₁₂ alkylene).

In embodiments, L is optionally substituted 2- to 12-membered heteroalkylene.

In embodiments, linker L is —(CH₂)ₙ—, and n is 2, 3, 4, or 5. In embodiments, n is 2. In embodiments, n is 3. In embodiments, n is 4. In embodiments, n is 5.

In embodiments, the compound has the following structure,

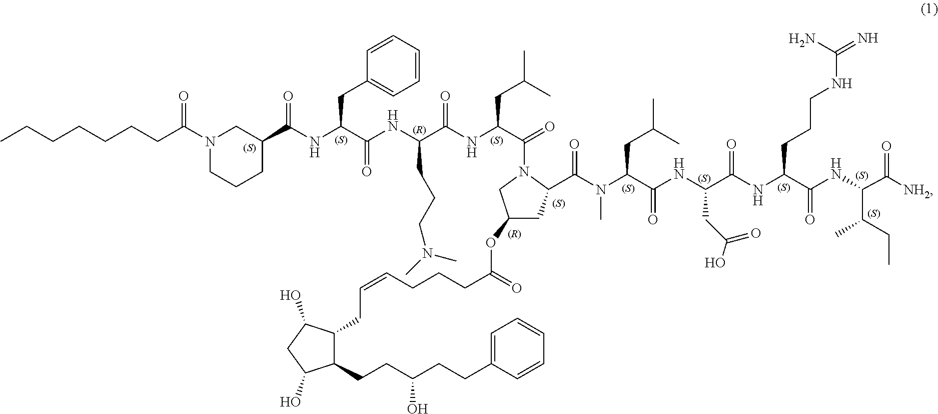

or a pharmaceutically acceptable salt thereof.

In one aspect, the invention features a composition comprising any compound described herein (e.g., a compound of any one of Formulas (I)-(V) such as Compound (1)).

In another aspect, the invention features a pharmaceutical composition comprising any compound described herein (e.g., a compound of any one of Formulas (I)-(V) such as Compound (1)), and a pharmaceutically acceptable excipient.

In embodiments, the composition comprises the compound in an amount that is about 0.001% (w/w) to about 1.000% (w/w), about 0.001% (w/w) to about 0.500% (w/w), about 0.001% (w/w) to about 0.250% (w/w), about 0.001% to about 0.150%, about 0.001% to about 0.100%, about 0.001% to about 0.090%, about 0.001% to about 0.075%, about 0.001% to about 0.050%, or about 0.001% to about 0.010%.

In embodiments, the composition comprises the compound in an amount that is about 0.005% (w/w) to about 1.000% (w/w), about 0.005% (w/w) to about 0.500% (w/w), about 0.005% (w/w) to about 0.250% (w/w), about 0.005% to about 0.150%, about 0.005% to about 0.100%, about 0.005% to about 0.090%, about 0.005% to about 0.075%, about 0.005% to about 0.050%, or about 0.005% to about 0.010%.

In embodiments, the composition comprises the compound in an amount that is about 0.010% (w/w) to about 2.000% (w/w), about 0.010% (w/w) to about 1.500% (w/w), about 0.010% (w/w) to about 1.000% (w/w), about 0.010% (w/w) to about 0.900% (w/w), about 0.010% (w/w) to about 0.800% (w/w), about 0.010% (w/w) to about 0.700% (w/w), about 0.010% (w/w) to about 0.600% (w/w), about 0.010% (w/w) to about 0.500% (w/w), about 0.010% (w/w) to about 0.250% (w/w), about 0.010% to about 0.150%, about 0.010% to about 0.100%, about 0.010% to about 0.090%, about 0.010% to about 0.075%, or about 0.010% to about 0.050%.

In embodiments, the composition comprises the compound in an amount that is about 0.050% (w/w) to about 2.000% (w/w), about 0.050% (w/w) to about 1.500% (w/w), about 0.050% (w/w) to about 1.000% (w/w), about 0.050% (w/w) to about 0.500% (w/w), about 0.050% (w/w) to about 0.250% (w/w), about 0.050% (w/w) to about 0.200% (w/w), about 0.050% to about 0.150%, or about 0.050% to about 0.125%.

In embodiments, the composition comprises the compound in an amount that is about 0.075% (w/w) to about 2.000% (w/w), about 0.075% (w/w) to about 1.500% (w/w), about 0.075% (w/w) to about 1.250% (w/w), about 0.075% (w/w) to about 1.000% (w/w), about 0.075% (w/w) to about 0.750% (w/w), about 0.075% (w/w) to about 0.500% (w/w), about 0.075% (w/w) to about 0.250% (w/w), about 0.075% (w/w) to about 0.200% (w/w), or about 0.075% (w/w) to about 0.150% (w/w).

### Combinations of NPR-B Agonists and Prostaglandin Agonists

In one aspect, the invention features a composition comprising:
a first compound that is a linear peptidic NPR-B agonist, or a pharmaceutically acceptable salt thereof; and
a second compound that is a prostaglandin agonist or a β-adrenergic antagonist, or a pharmaceutically acceptable salt thereof.

In embodiments, a second compound is a prostaglandin agonist.

In embodiments, a second compound is a β-adrenergic antagonist.

In another aspect, the invention features a pharmaceutical composition comprising:
a first compound that is a linear peptidic NPR-B agonist, or a pharmaceutically acceptable salt thereof;
a second compound that is a prostaglandin agonist or a β-adrenergic antagonist, or a pharmaceutically acceptable salt thereof; and
a pharmaceutically acceptable excipient.

In embodiments, a second compound is a prostaglandin agonist.

In embodiments, a second compound is a β-adrenergic antagonist. In another aspect, the invention features a method of treating a patient in need thereof, said method comprising co-administering to said patient: a first compound that is a linear peptidic NPR-B agonist, or a pharmaceutically acceptable salt thereof; and a second compound that is a prostaglandin agonist or a β-adrenergic antagonist, or a pharmaceutically acceptable salt thereof.

In embodiments, a second compound is a prostaglandin agonist.

In embodiments, a second compound is a β-adrenergic antagonist.

In embodiments, a second composition comprises travoprost in an amount that is about 0.001% (w/w) to about 0.05% (w/w). In embodiments, travoprost is present in an amount that is about 0.005% (w/w) to about 0.05% (w/w). In embodiments, travoprost is present in an amount that is about 0.01% (w/w) to about 0.05% (w/w). In embodiments, travoprost is present in an amount that is about 0.01% (w/w). In embodiments, travoprost is present in an amount that is about 0.02% (w/w). In embodiments, travoprost is present in an amount that is about 0.03% (w/w). In embodiments, travoprost is present in an amount that is about 0.04% (w/w). In embodiments, travoprost is present in an amount that is about 0.05% (w/w).

In embodiments, said first compound and said second compound are administered simultaneously. In embodiments, a single pharmaceutical formulation comprises said first compound, or a pharmaceutically acceptable salt thereof, and said second compound, or a pharmaceutically acceptable salt thereof.

In embodiments, said first compound and said second compound are administered contemporaneously (e.g., said first compound is administered before, during, or after administration of said second compound).

In embodiments, said first compound and said second compound are administered in separate pharmaceutical compositions (e.g., a first pharmaceutical composition comprising said first compound, or a pharmaceutically acceptable salt thereof, and a second pharmaceutical composition comprising said second compound, or a pharmaceutically acceptable salt thereof).

Thus, in embodiments, the invention also features methods of administering to a patient in need thereof:
a first composition (e.g., a pharmaceutical composition) comprising a first compound that is a linear peptidic NPR-B agonist, or a pharmaceutically acceptable salt thereof;
a second composition (e.g., a pharmaceutical composition) comprising a second compound that is a prostaglandin agonist or a β-adrenergic antagonist, or a pharmaceutically acceptable salt thereof.

In embodiments, a linear peptidic NPR-B agonist is a peptide as described in, e.g., U.S. Pat. No. 8,546,523 or International Publication No. WO 2011/038061, each of which is hereby incorporated by reference in its entirety.

In embodiments, a linear peptidic NPR-B agonist is a compound according to the following formula (A),

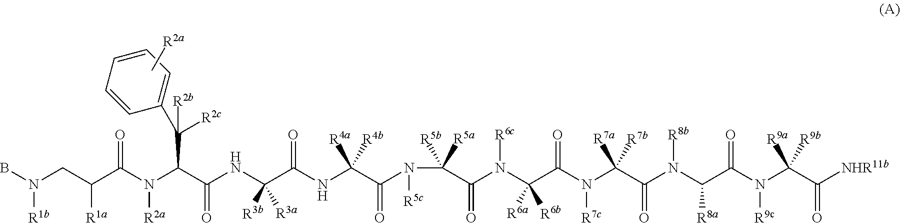

wherein

B is selected from the group consisting of R^{b1}— and R^{b2}—C(O)—, wherein:
R^{b1} is selected from the group consisting of C₆-C₁₀ alkyl and C₆-C₁₀ alkyl substituted by NR^{b4}R^{b5};
R^{b2} is selected from the group consisting of C₆-C₁₀ alkyl and C₆-C₁₀ alkyl substituted by NR^{b4}R^{b5}; and
R^{b4} and R^{b5} are, independently, selected from the group consisting of H and C₁-C₄ alkyl;

R^{1a} is selected from H and C₁-C₄ alkyl; and

R^{1b} is selected from H, C₁-C₄ alkyl optionally substituted by OH, and hydroxy C₁-C₄ alkyl optionally substituted by OH; or alternatively,

R^{1a} and R^{1b} together form a heterocyclic ring;

n¹ is 0 or 1;

R^{2a} is selected from the group consisting of H, methyl, ethyl, propyl, isopropyl, C₁-C₂ alkyl C₃-C₇ cycloalkyl and aryl C₁-C₂ alkyl;

R^{2b} and R^{2c} are, independently, selected from the group consisting of H, methyl, ethyl, propyl, and isopropyl, with the proviso that at least one of R^{2b} and R^{2c} is H;

R^{2d} represents from 0 to 3 substituents, each such substituent being, independently, selected from the group consisting of H, Cl, F, Br, CN, CF₃, OH, OR^{2e} and C₁-C₄ alkyl, wherein R^{2e} is selected from the group consisting of methyl, ethyl, propyl, and isopropyl;

R^{3a} is selected from the group consisting of H and C₁-C₄ alkyl; and

R^{3b} is selected from the group consisting of H, and —(CH₂)ₙ₃ₐ—X^{3a}, wherein
n3a is 1 to 5; and
X^{3a} is selected from the group consisting of H and NR^{3c}R^{3d}, wherein R^{3c} and R^{3d} are independently selected from the group consisting of H, C₁-C₈ alkyl, and —(C═N)—NH₂; or alternatively,

R^{3a} and R^{3b} are linked to form a cyclic structure; or alternatively,

R^{3a} and R^{3b} are linked with a heteroatom selected from the group consisting of N, O, and S, to form a heterocyclic structure;

R^{4a} is selected from the group consisting of H, and C₁-C₈ alkyl which is optionally substituted with a moiety selected from the group consisting of OH, and CO₂R^{4c}, wherein Fe′ is selected from the group consisting of H, and C₁-C₃alkyl;

R^{4b} is selected from the group consisting of H and methyl;

R^{5a} is (CH₂)ₙ₅ₐ—X^{5a}, wherein
n5a is 1 to 6; and
X^{5a} is selected from the group consisting of H, NH₂, and a C₄-C₇ amine-containing aliphatic heterocyclic ring; and

R^{5c} is selected from the group consisting of H and methyl; or alternatively,

R^{5c} and R^{5a} combine to form a4 to 6 membered heterocyclic ring, wherein said heterocyclic ring has from 0 to 2 substituents, each such substituent independently selected from the group consisting of OH, F, C₁-C₄ alkyl, —NHC(═NH)NH₂, aryl, and NR^{5e}R^{5f}, wherein
R^{5e} is selected from the group consisting of H, C₁-C₄ alkyl, —C(═O)(CH₂)_{n5b}—X^{5b}, and —CH₂(CH₂)_{n5c}—X^{5b}; and
R^{5f} is selected from the group consisting of H, C₁-C₄ alkyl, and —CH₂(CH₂)_{n5d}—X^{5c}; wherein
   n5b is selected from the group consisting of 1, 2, 3, and 4;
   n5c and n5dare independently selected from the group consisting of 2, 3, and 4; and
   X^{5b} and X^{5c} are independently selected from the group consisting of H and NR^{5g}R^{5h}; wherein R^{5g} and R^{5h} are independently selected from the group consisting of H and C₁-C₄ alkyl;

R^{5b} is selected from the group consisting of H and methyl;

R^{6a} is selected from the group consisting of C₁-C₈ alkyl, aryl C₁-C₄ alkyl, C₄-C₇ cycloalkyl C₁-C₄ alkyl, and C₄-C₇ cycloalkyl, wherein each of said C₁-C₈ alkyl and C₄-C₇ cycloalkyl is optionally substituted with a moiety selected from the group consisting of OH, and O(C₁-C₄ alkyl);

R^{6b} is H;

R^{6c} is selected from the group consisting of H and C₁-C₄alkyl;

R^{7a} is selected from the group consisting of C₁-C₄alkyl, C₃-C₂cycloalkyl, 2-thienyl, (CH₂)ₙ₇ₐ—X^{7a}, and C₁-C₄alkyl substituted with OH, wherein
n^{7a} is selected from the group consisting of 1 and 2; and
X^{7a} is selected from the group consisting of 2-thienyl, C(═O)OR^{7e}, C(═O)NH₂, S(═O)₂OH, OS(═O)₂OH, B(OH)₂, P(═O)(OH)₂, and OP(═O)(OH)₂, wherein R^{7e} is selected from the group consisting of H and C₁-C₄alkyl;

R^{7b} is H or 2-thienyl;

R^{7c} is selected from the group consisting of H and methyl;

R^{8a} is (CH₂)ₘ₈ₐ—X^{8a}, wherein
m^{8a}=₁₋₅;
X^{8a} is selected from the group consisting of H, NH₂, and —NHC(═NH)NH₂;

R^{8b} is selected from the group consisting of H and methyl;

R^{9a} is selected from the group consisting of C₁-C₅alkyl and C₄-C₇ cycloalkyl; and

R^{9b} is selected from the group consisting of H and C₁-C₅alkyl; or alternatively,

R^{9a} and R^{9b} form a 5-7 membered cycloalkyl ring;

R^{9c} is selected from the group consisting of H and methyl; and

R^{11b} is selected from the group consisting of H, C₁-C₈ alkyl, C₄-C₈ cycloalkyl, C₇-C₁₂ bicycloalkyl, C₇-C₁₂ cycloalkylaryl, and C₁-C₄ alkyl-C₄-C₈ cycloalkyl.

In embodiments, a linear peptidic NPR-B agonist is a compound of formula (B-1),

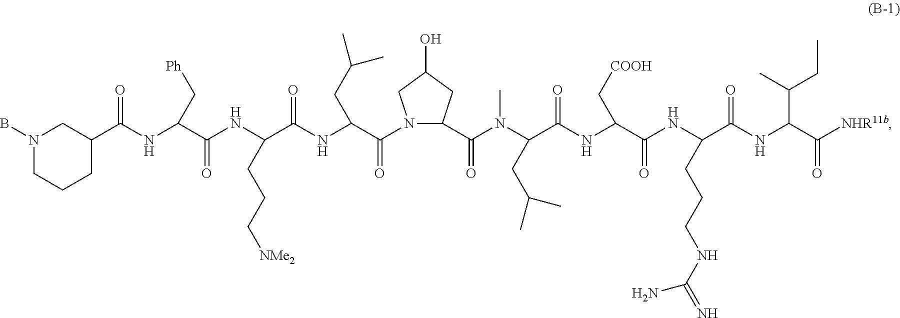

or a pharmaceutically acceptable salt thereof, wherein

B is selected from the group consisting of R^{b1}— and R^{b2}—C(O)—;

R^{b1} is selected from the group consisting of C₆-C₁₀ alkyl and C₆-C₁₀ alkyl substituted by NR^{b4}R^{b5};

R^{b2} is selected from the group consisting of C₆-C₁₀ alkyl and C₆-C₁₀ alkyl substituted by NR^{b4}R^{b5};

R^{b4} and R^{b5} are, independently, selected from the group consisting of H and C₁-C₄ alkyl; and

R^{11b} is selected from the group consisting of H, C₁-C₈ alkyl, C₄-C₈ cycloalkyl, C₇-C₁₂ bicycloalkyl, and C₁-C₄ alkyl-C₄-C₈ cycloalkyl.

In embodiments, a compound of formula (B-1) is a compound of formula (B-2),

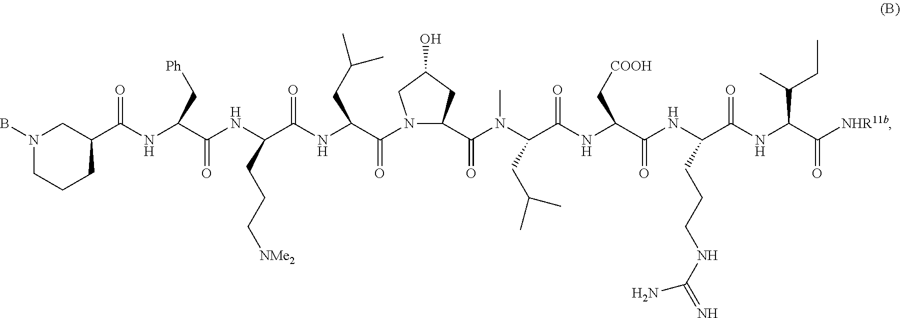

or a pharmaceutically acceptable salt thereof, wherein

B is selected from the group consisting of R^{b1}— and R^{b2}—C(O)—;

R^{b1} is selected from the group consisting of C₆-C₁₀ alkyl and C₆-C₁₀ alkyl substituted by NR^{b4}R^{b5};

R^{b2} is selected from the group consisting of C₆-C₁₀ alkyl and C₆-C₁₀ alkyl substituted by NR^{b4}R^{b5};

R^{b4} and R^{b5} are, independently, selected from the group consisting of H and C₁-C₄ alkyl; and

R^{11b} is selected from the group consisting of H, C₁-C₈ alkyl, C₄-C₈cycloalkyl, C₂-C₁₂ bicycloalkyl, and C₁-C₄ alkyl-C₄-C₈ cycloalkyl.

In embodiments, B is R^{b2}—C(O)— and R^{b2} is C₆-C₁₀ alkyl; and R^{11b} is selected from the group consisting of H and C₁-C₈ alkyl.

In embodiments, a linear peptidic NPR-B agonist or pharmaceutically acceptable salt thereof has the following sequence,
Occ-Sni-Phe-orn(Me2)-Leu-Hyp-Nml-Asp-Arg-Ile-NH₂ (SEQ ID NO:1);
   wherein

“Occ” represents a moiety formed from octanoic acid;

“Sni” represents a residue formed from (S)-nipecotic acid;

“orn(Me2)” represents a residue formed form (R)-2-amino-5-(dimethylamino) pentanoic acid;

“Leu” is a standard abbreviation and represents a residue formed from leucine;

“Hyp” represents a residue formed form (2S,4R)-4-hydroxypyrrolidine-2-carboxylic acid;

“Nml” represents a residue formed from (S)—N-methyl-leucine;

“Asp” is a standard abbreviation and represents a residue formed from aspartic acid;

“Arg” is a standard abbreviation and represents a residue formed from arginine; and

“Ile” is a standard abbreviation and represents a residue formed from isoleucine.

The peptide of SEQ ID NO:1, or a pharmaceutically acceptable salt thereof, is also represented by the following chemical structure,

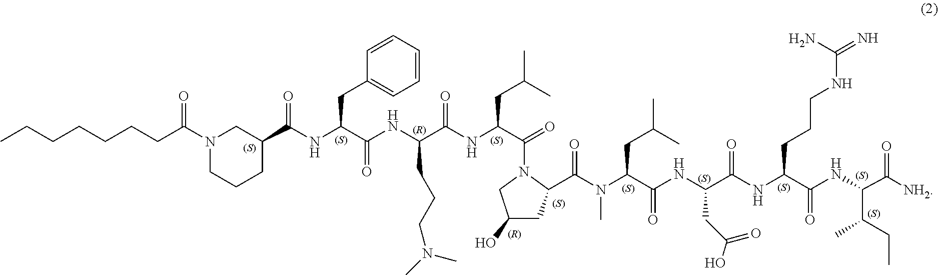

In embodiments, a composition comprises a linear peptidic NPR-β agonist. In embodiments, a composition comprising a linear peptidic NPR-β agonist does not comprise any prostaglandin agonists or any β-adrenergic antagonists.

In embodiments, a composition comprises a linear peptidic NPR-β agonist in an amount that is about 0.01% (w/w) to about 0.5% (w/w). In embodiments a composition comprises a linear peptidic NPR-β agonist in an amount that is about 0.01% (w/w) to about 0.20% (w/w). In embodiments, a composition comprises a linear peptidic NPR-β agonist in an amount that is about 0.01% (w/w) to about 0.15% (w/w). In embodiments, a composition comprises a linear peptidic NPR-β agonist in an amount that is about 0.03% (w/w). In embodiments, a composition comprises a linear peptidic NPR-β agonist in an amount that is about 0.1% (w/w). In embodiments, a composition comprises a linear peptidic NPR-β agonist in an amount that is about 0.6% (w/w). In embodiments, a linear peptidic NPR-β agonist is a compound of Formula (B-1) such as SEQ ID NO:1. In embodiments, a linear peptidic NPR-β agonist is a compound of SEQ ID NO:1.

In embodiments, a composition (e.g., a pharmaceutical composition) comprises one and only one prostaglandin agonist.

In embodiments, a composition (e.g., a pharmaceutical composition) comprises a mixture of prostaglandin agonists (e.g., 1, 2, 3, or 4 different prostaglandin agonists).

In embodiments, a composition (e.g., a pharmaceutical composition) comprises one and only one β-adrenergic antagonist.

In embodiments, a composition (e.g., a pharmaceutical composition) comprises a mixture of prostaglandin agonists (e.g., 1, 2, 3, or 4 different β-adrenergic antagonists).

In embodiments, a composition (e.g., a pharmaceutical composition) comprises a prostaglandin agonist and a β-adrenergic antagonist

In embodiments, a second compound is a prostaglandin agonist that reduces intraocular pressure (IOP) in a subject in need thereof.

In embodiments, a second compound is a prostaglandin agonist suitable for treating or preventing glaucoma in a patient in need thereof.

In embodiments, a second compound is a prostaglandin agonist that is Xalatan® (latanoprost), Lumigan® (bimatoprost), Travatan Z® (travoprost), or Zioptan™ (tafluprost).

In embodiments, a second compound is a prostaglandin agonist that is Xalatan® (latanoprost). In embodiments, a composition comprises latanoprost in an amount that is about 0.001% (w/w) to about 0.01% (w/w). In embodiments, a composition comprises latanoprost in an amount that is about 0.002% (w/w). In embodiments, a composition comprises latanoprost in an amount that is about 0.003% (w/w). In embodiments, a composition comprises latanoprost in an amount that is about 0.004% (w/w). In embodiments, a composition comprises latanoprost in an amount that is about 0.005% (w/w). In embodiments, a composition comprises latanoprost in an amount that is about 0.006% (w/w).

In embodiments, a second compound is a prostaglandin agonist that is Lumigan® (bimatoprost). In embodiments, a composition comprises bimatoprost in an amount that is about 0.01% (w/w) to about 0.1% (w/w). In embodiments, a composition comprises bimatoprost in an amount that is about 0.01% (w/w). In embodiments, a composition comprises bimatoprost in an amount that is about 0.02% (w/w). In embodiments, a composition comprises bimatoprost in an amount that is about 0.03% (w/w). In embodiments, a composition comprises bimatoprost in an amount that is about 0.04% (w/w). In embodiments, a composition comprises bimatoprost in an amount that is about 0.05% (w/w).

In embodiments, a second compound is a prostaglandin agonist that is Travatan Z® (travoprost). In embodiments, a composition comprises travoprost in an amount that is about 0.001% (w/w) to about 0.01% (w/w). In embodiments, a composition comprises travoprost in an amount that is about 0.002% (w/w). In embodiments, a composition comprises travoprost in an amount that is about 0.003% (w/w). In embodiments, a composition comprises travoprost in an amount that is about 0.004% (w/w). In embodiments, a composition comprises travoprost in an amount that is about 0.005% (w/w). In embodiments, a composition comprises travoprost in an amount that is about 0.006% (w/w).

In embodiments, a second compound is a prostaglandin agonist that is (tafluprost).

In embodiments, a second compound or pharmaceutically acceptable salt thereof has a structure according to the following formula,

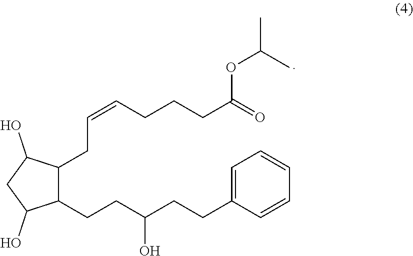 [SMILES: CC(C)OC(=O)CCC/C=C\CC1C(O)CC(O)C1CCC(O)CCc1ccccc1]

In embodiments, a second compound or pharmaceutically acceptable salt thereof has a structure according to the following formula,

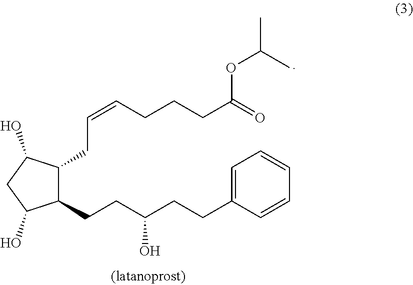 [SMILES: CC(C)OC(=O)CCC/C=C\C[C@@H]1[C@@H](CC[C@@H](O)CCc2ccccc2)[C@H](O)C[C@@H]1O]

In embodiments, a second compound or pharmaceutically acceptable salt thereof has a structure according to the following formula,

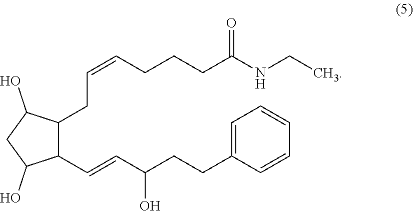 [SMILES: CCNC(=O)CCC/C=C\CC1C(O)CC(O)C1/C=C/C(O)CCc1ccccc1]

In embodiments, a second compound or pharmaceutically acceptable salt thereof has a structure according to the following formula,

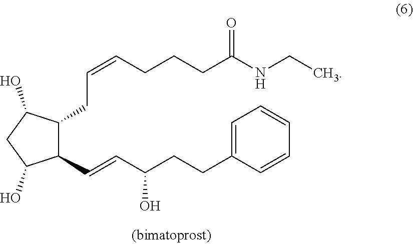 [SMILES: CCNC(=O)CCC/C=C\C[C@@H]1[C@@H](/C=C/[C@@H](O)CCc2ccccc2)[C@H](O)C[C@@H]1O]

In embodiments, a second compound or pharmaceutically acceptable salt thereof has a structure according to the following formula,

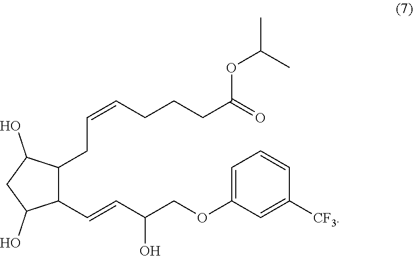 [SMILES: CC(C)OC(=O)CCC/C=C\CC1C(O)CC(O)C1/C=C/C(O)COc1cccc(C(F)(F)F)c1]

In embodiments, a second compound or pharmaceutically acceptable salt thereof has a structure according to the following formula,

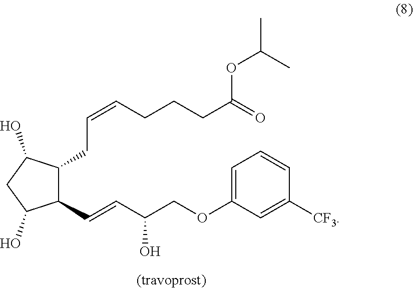 [SMILES: CC(C)OC(=O)CCC/C=C\C[C@@H]1[C@@H](/C=C/[C@@H](O)COc2cccc(C(F)(F)F)c2)[C@H](O)C[C@@H]1O]

In embodiments, a second compound or pharmaceutically acceptable salt thereof has a structure according to the following formula,

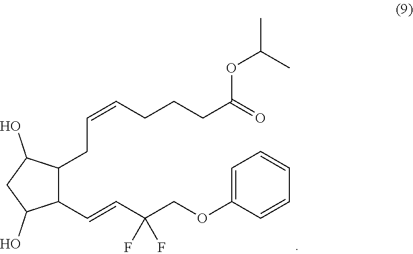 [SMILES: CC(C)OC(=O)CCC/C=C\CC1C(O)CC(O)C1/C=C/C(F)(F)COc1ccccc1]

In embodiments, a second compound or pharmaceutically acceptable salt thereof has a structure according to the following formula,

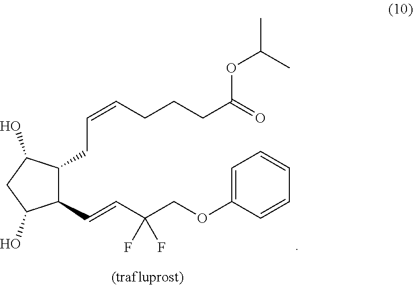 [SMILES: CC(C)OC(=O)CCC/C=C\C[C@@H]1[C@@H](/C=C/C(F)(F)COc2ccccc2)[C@H](O)C[C@@H]1O]

In embodiments, a second compound is a β-adrenergic antagonist.

In embodiments, a second compound is betaxolol, carteolol, levobunolol, metipranolol, or timolol (e.g., timolol maleate or timolol hemihydrate).

In embodiments, a second compound is betaxolol.

In embodiments, a second compound is carteolol. In embodiments, a second compound is levobunolol.

In embodiments, a second compound is metipranolol.

In embodiments, a second compound is timolol (e.g., timolol maleate or timolol hemihydrate).

In embodiments, a composition comprises

a first compound having the structure,

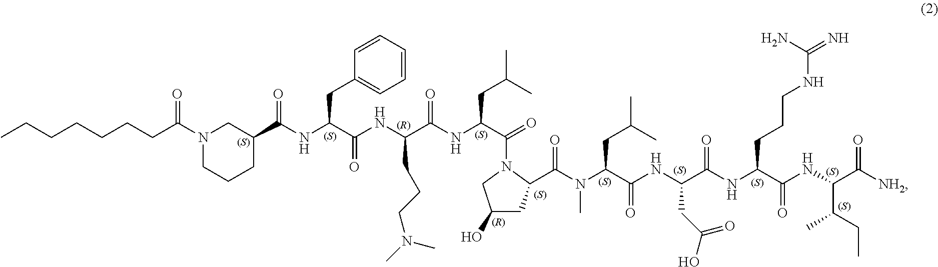

or a pharmaceutically acceptable salt thereof; and

a second compound that is a prostaglandin agonist or a β-adrenergic antagonist.

In embodiments, a second compound is a prostaglandin agonist.

In embodiments, a second compound is a β-adrenergic antagonist.

In embodiments, a pharmaceutical composition comprises

a first compound having the structure,

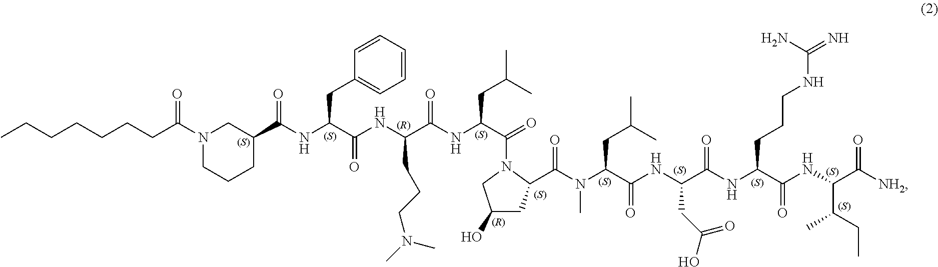

or a pharmaceutically acceptable salt thereof;

a second compound that is a prostaglandin agonist or a β-adrenergic antagonist; and

a pharmaceutically acceptable excipient.

In embodiments, a second compound is a prostaglandin agonist.

In embodiments, a second compound is a β-adrenergic antagonist.

In embodiments, said first compound, or a pharmaceutically acceptable salt thereof, is present in an amount that is about 0.001% (w/w) to about 2.000% (w/w), about 0.010% (w/w) to about 1.500% (w/w), about 0.010% (w/w) to about 1.000%, about 0.010% (w/w) to about 0.900% (w/w), about 0.010% (w/w) to about 0.800% (w/w), about 0.010% (w/w) to about 0.700% (w/w), about 0.010% (w/w) to about 0.600% (w/w), about 0.010% (w/w) to about 0.500% (w/w), about 0.010% (w/w) to about 0.400% (w/w), about 0.010% (w/w) to about 0.300% (w/w), about 0.010% (w/w) to about 0.200% (w/w), about 0.010% (w/w) to about 0.15% (w/w), about 0.020% (w/w) to about 0.200% (w/w), about 0.020% (w/w) to about 0.150% (w/w), about 0.050% (w/w) to about 0.200% (w/w), or about 0.050% (w/w) to about 0.150% (w/w). In embodiments, a first compound is present in an amount that is about 0.03% (w/w), about 0.1% (w/w), or about 0.6% (w/w). In embodiments, a first compound is present in an amount that is about 0.03% (w/w). In embodiments, a first compound is present in an amount that is about 0.1% (w/w). In embodiments, a first compound is present in an amount that is about 0.6% (w/w).

In embodiments, said second compound is present in an amount that is about 0.001% (w/w) to about 0.500% (w/w), about 0.001% (w/w) to about 0.250% (w/w), about 0.001% to about 0.100%, about 0.001% to about 0.090%, about 0.001% to about 0.075%, about 0.001% to about 0.050%, or about 0.001% to about 0.010%.

In embodiments, said first compound, or a pharmaceutically acceptable salt thereof, is present in an amount that is about 0.01% (w/w) to about 0.20% (w/w), 0.05% (w/w) to about 0.20% (w/w), or about 0.05% (w/w) to about 0.15% (w/w); and said second compound is present in an amount that is about 0.001% (w/w) to about 0.100% (w/w), or about 0.001% (w/w) to about 0.010% (w/w).

In embodiments, said first compound, or a pharmaceutically acceptable salt thereof, is present in an amount that is about 0.05% (w/w) to about 0.15% (w/w); and said second compound is present in an amount that is about 0.001% (w/w) to about 0.010% (w/w).

In embodiments, the relative (w/w) ratio of said first compound to said second compound is about 100 to about 1; about 50 to about 1; about 40 to about 1; about 30 to about 1; about 20 to about 1; about 10 to about 1; about 1 to about 1; about 1 to about 10; or about 1 to about 20.

In embodiments, the relative (w/w) ratio of said first compound to said second compound is about 40 to about 1; about 35 to about 1; about 30 to about 1; about 25 to about 1; about 20 to about 1; about 15 to about 1; about 10 to about 1; or about 5 to about 1.

In embodiments, the relative (w/w) ratio of said first compound to said second compound is about 30 to about 1; about 25 to about 1; about 20 to about 1; or about 15 to about 1.

### Pharmaceutical Compositions

As provided herein, exemplary compositions of the invention include pharmaceutical compositions comprising any compound described herein (e.g., a compound of any one of Formulas (I)-(V) such as Compound (1)), and a pharmaceutically acceptable excipient.

As provided herein, exemplary compositions of the invention include pharmaceutical compositions comprising: a first compound that is a linear peptidic NPR-B agonist, or a pharmaceutically acceptable salt thereof; a second compound that is a prostaglandin agonist, or a pharmaceutically acceptable salt thereof; and a pharmaceutically acceptable excipient.

As provided herein, exemplary compositions of the invention include a first pharmaceutical composition comprising a first compound that is a linear peptidic NPR-B agonist, or a pharmaceutically acceptable salt thereof, and a pharmaceutically acceptable excipient; and a second pharmaceutical composition comprising a second compound that is a prostaglandin agonist, or a pharmaceutically acceptable salt thereof, and a pharmaceutically acceptable excipient.

In embodiments, a pharmaceutical composition described herein can be formulated in any manner known to those of ordinary skill in the art. In embodiments, the concentration of an individual compound can be any concentration known or suspected by those skilled in the art to be of benefit in the treatment and/or prevention of ophthalmic disease associated with elevated intraocular pressure or ocular hypertension. In embodiments, the total concentration of the combined therapeutic agents can be any concentration known or suspected by those skilled in the art to be of benefit in the treatment and/or prevention of ophthalmic disease associated with elevated intraocular pressure or ocular hypertension.

In embodiments, a pharmaceutical composition comprises any ingredient listed in Tables 1-5, or any combination thereof. In embodiments, a pharmaceutical composition is the composition described in Table 1. In embodiments, a pharmaceutical composition is the composition described in Table 2. In embodiments, a pharmaceutical composition is the composition described in Table 3. In embodiments, a pharmaceutical composition is the composition described in Table 4. In embodiments, a pharmaceutical composition is the composition described in Table 5. In embodiments, any specified value or any specified range described in any of Tables 1-5 can vary by about 1-20%, about 1-15%, or about 1-5% in either direction.

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

A dose may be any amount of a compound or composition (e.g., a pharmaceutical composition) that is known or suspected to be of therapeutic benefit. For example, a dose may be about 1 microgram/kg body weight to about 500 microgram/kg body weight or more per administration, and any range derivable therein. A dose may be repeated as necessary as determined by one of ordinary skill in the art to achieve a desired therapeutic effect. For example, a dose may be repeated once, twice, three times, and so forth. In embodiments, a dose is administered twice a day, three times a day, four times a day, or more often. In embodiments, a dose is administered every other day, twice a week, once a month, or at a longer interval.

In embodiments, administering of a compound or composition (e.g., a pharmaceutical composition) occurs one, two, three, or four times daily. In embodiments, administering of a compound or composition (e.g., a pharmaceutical composition) occurs once daily.

In embodiments, a composition (e.g., a pharmaceutical composition) can include more than one NPR-B agonist. One skilled in the art would be familiar with preparing and administering pharmaceutical compositions that include more than one therapeutic agent. In embodiments, the composition includes one or more additional therapeutic agents that are not NPR-B agonists.

In addition to the NPR-B agonists, the compositions of the present invention optionally comprise one or more excipients. Excipients commonly used in pharmaceutical compositions include carriers, tonicity agents, preservatives, chelating agents, buffering agents, surfactants and antioxidants.

In embodiments, a pharmaceutic composition comprises a viscosity enhancing agent.

In embodiments the viscosity enhancing agent is hydroxypropyl methylcellulose (HPMC).

In embodiments, HPMC is present in an amount that is about 0.05% (w/w) to about 0.75% (w/w). In embodiments, HPMC is present in an amount that is about 0.25% (w/w) to about 0.6% (w/w). In embodiments, HPMC is present in an amount that is 0.5 (w/w).

In embodiments, a pharmaceutic composition comprises a preservative.

In embodiments, the preservative is benzalkonium chloride (BAK).

In embodiments, BAK is present in an amount that is about 0.001% (w/w) to about 0.2% (w/w). In embodiments, BAK is present in an amount that is about 0.005% (w/w) to about 0.1% (w/w). In embodiments, BAK is present in an amount that is 0.01 (w/w).

In embodiments, a pharmaceutic composition comprises a chelating agent.

In embodiments, the chelating agent is ethylenediaminetetraacetic acid (EDTA).

In embodiments, EDTA is present in an amount that is about 0.001% (w/w) to about 0.2% (w/w). In embodiments, EDTA is present in an amount that is about 0.005% (w/w) to about 0.1% (w/w). In embodiments, EDTA is present in an amount that is 0.01 (w/w).

In embodiments, a pharmaceutic composition comprises a surfactant.

In embodiments, the surfactant is polysorbate 80.

In embodiments, polysorbate 80 is present in an amount that is about 0.001% (w/w) to about 0.2% (w/w). In embodiments, polysorbate 80 is present in an amount that is about 0.005% (w/w) to about 0.1% (w/w). In embodiments, polysorbate 80 is present in an amount that is 0.05 (w/w).

In embodiments, a pharmaceutic composition comprises a buffering agent.

In embodiments, the buffering agent is dibasic sodium phosphate.

In embodiments, dibasic sodium phosphate is present in an amount that is about 0.01% (w/w) to about 0.75% (w/w). In embodiments, dibasic sodium phosphate is present in an amount that is about 0.1% (w/w) to about 0.6% (w/w). In embodiments, dibasic sodium phosphate is present in an amount that is 0.2 (w/w).

In embodiments, a pharmaceutic composition comprises a tonicity agent.

In embodiments, the tonicity agent is sodium chloride (NaCl).

In embodiments, NaCl is present in an amount that is about 0.1% (w/w) to about 0.9% (w/w). In embodiments NaCl is present in an amount that is about 0.3% (w/w) to about 0.8% (w/w). In embodiments, NaCl is present in an amount that is 0.75 (w/w).

In embodiments, a pharmaceutical composition comprises a pH adjusting agent.

In embodiments, a pharmaceutic composition comprises a linear peptidic NPR-B agonist (e.g., a compound of Formula (B) such as SEQ ID NO:1) in an amount that is about 0.05% (w/w) to about 0.75% (w/w); a prostaglandin agonist (e.g., latanoprost) in an amount that is about 0.001% (w/w) to about 0.01% (w/w); a chelating agent (e.g., EDTA) in an amount that is about 0.005% to about 0.1% (e.g., about 0.01%); a viscosity increasing agent (e.g., HPMC) in an amount that is about 0.05% (w/w) to about 0.75% (w/w); a preservative (e.g. BAK) in an amount that is about 0.001% (w/w) to about 0.2% (w/w);); a surfactant (e.g. polysorbate 80) in an amount that is about 0.001% (w/w) to about 0.2% (w/w); a phosphate buffer (e.g. dibasic sodium phosphate) present in an amount that is about 0.01% (w/w) to about 0.75% (w/w); and a tonicity agent (e.g. NaCl) in an amount that is about 0.1% (w/w) to about 0.9% (w/w).

A person skilled in the art will recognize that the compositions of the present invention can include any number of combinations of ingredients (e.g., active agent, polymers, excipients, etc.). It is also contemplated that that the concentrations of these ingredients can vary. In some aspects, the percentage of each ingredient in the composition can be calculated by weight or volume of the total composition. A person skilled in the art would understand that the concentrations can vary depending on the addition, substitution, and/or subtraction of particular ingredients in a given composition.

Any of a variety of carriers may be used in the formulations of the present invention including water, mixtures of water and water-miscible solvents, such as C1-7-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, mixtures of those polymers. The concentration of the carrier is, typically, from 1 to 100000 times the concentration of the active ingredient.

Suitable tonicity-adjusting agents include mannitol, sodium chloride (NaCl), glycerin, sorbitol and the like. Suitable preservatives include p-hydroxybenzoic acid ester, benzalkonium chloride (BAK), benzododecinium bromide, polyquaternium-1 and the like. Suitable chelating agents include sodium edetate and the like. Suitable buffering agents include phosphates, borates, citrates, acetates and the like. Suitable surfactants include ionic and nonionic surfactants, though nonionic surfactants are preferred, such as polysorbates, polysorbate 80, polyethoxylated castor oil derivatives and oxyethylated tertiary octylphenol formaldehyde polymer (tyloxapol). Suitable antioxidants include sulfites, ascorbates, BHA and BHT. The compositions of the present invention optionally comprise an additional active agent.

Suitable pH adjusting agents include lactic acid, citric acid, tartaric acid, phosphoric acid, acetic acid, hydrochloric acid, nitric acid, sodium or potassium metaphosphate, sodium or potassium phosphate, sodium or potassium acetate, ammonia, sodium carbonate, sodium or potassium hydroxide, dibasic sodium phosphate, sodium borate, and the like and mixtures thereof.

In embodiments, a pharmaceutical composition is formulated for ophthalmic use. In embodiments, the compositions are suitable for application to eyes (e.g., mammalian eyes). For example, for ophthalmic administration, the formulation may be a solution, a suspension, a gel, or an ointment, or any other suitable formulation.

In embodiments, a pharmaceutical composition (e.g., an ophthalmic pharmaceutical formulation) is formulated for topical administration.

In embodiments, a pharmaceutical formulation will be formulated for topical application to the eye in aqueous solution in the form of drops.

In embodiments, components of the invention may be delivered to the eye as a concentrated gel or similar vehicle which forms dissolvable inserts that are placed beneath the eyelids.

In embodiments, a composition of the present invention may also be formulated as solutions that undergo a phase transition to a gel upon administration to the eye.

In embodiments, a pharmaceutical composition is formulated as an injectable pharmaceutical composition.

In embodiments, a pharmaceutical composition is formulated as an injectable ophthalmic pharmaceutical composition.

In embodiments, a pharmaceutical composition is formulated as a sustained or extended release pharmaceutical composition.

In embodiments, a pharmaceutical composition is formulated as a gel.

In embodiments, a pharmaceutical composition comprises drug-loaded microspheres.

In embodiments, a pharmaceutical composition is formulated as a depot implant.

In embodiments, a pharmaceutical composition comprises one or more polymers.

In embodiments, a pharmaceutical composition comprises collagen.

In embodiments, a pharmaceutical composition comprises a hydrogel (e.g., a hydrogel carrier).

In embodiments, a hydrogel comprises polyethylene glycol.

In embodiments, a hydrogel comprises one or more cellulosic polymers.

In embodiments, said hydrogel comprises hydroxypropyl methylcellulose (HPMC), hydroxypropyl cellulose (HPC), polycarbophil, or xanthan gum. In embodiments, said hydrogel comprises any combination of hydroxypropyl methylcellulose (HPMC), hydroxypropyl cellulose (HPC), polycarbophil, or xanthan gum. In embodiments, said hydrogel comprises a copolymer of hydroxypropyl methylcellulose (HPMC), hydroxypropyl cellulose (HPC), polycarbophil, or xanthan gum

Pharmaceutical compositions of the present invention may comprise other ingredients as excipients. For example, the compositions may include one or more pharmaceutically acceptable buffering agents, preservatives (including preservative adjuncts), non-ionic tonicity-adjusting agents, surfactants, solubilizing agents, stabilizing agents, comfort-enhancing agents, polymers, emollients, pH-adjusting agents and/or lubricants.

In embodiments, said pharmaceutical composition comprises one or more pharmaceutically acceptable excipients selected from tonicity agents, viscosity enhancing agents, buffering agents, pH adjusting agents, surfactants, preservatives, chelating agents, and combinations thereof.

The formulations set forth herein may comprise one or more preservatives. Examples of preservatives include quaternary ammonium compounds, such as benzalkonium chloride (BAK) or benzoxonium chloride. Other examples of preservatives include ethylenediaminetetraacetic acid (EDTA), alkyl-mercury salts of thiosalicylic acid such as thiomersal, phenylmercuric nitrate, phenylmercuric acetate or phenylmercuric borate; sodium perborate; sodium chlorite; parabens such as methylparaben or propylparaben; alcohols such as chlorobutanol, benzyl alcohol or phenyl ethanol; guanidine derivatives such as chlorohexidine or polyhexamethylene biguanide; sodium perborate; and sorbic acid.

In embodiments, a pharmaceutical composition further comprises one or more tear substitutes. A variety of tear substitutes are known in the art and include: monomeric polyols such as glycerol, propylene glycol, and ethylene glycol; polymeric polyols such as polyethylene glycol; cellulose esters such as hydroxypropylmethyl cellulose, carboxy methylcellulose sodium and hydroxy propylcellulose; dextrans such as dextran 70; water soluble proteins such as gelatin; vinyl polymers such as polyvinyl alcohol, polyvinylpyrrolidone, and povidone; and carbomers such as carbomer 934P, carbomer 941, carbomer 940 and carbomer 974P. The formulation of the present invention may be used with contact lenses or other ophthalmic products.

In embodiments, the compositions set forth herein have a viscosity of about 0.5 to about 10 cps, such as about 0.5 to about 5 cps or about 1 to about 2 cps. This relatively low viscosity ensures that the product is comfortable, does not cause blurring, and is easily processed during manufacturing, transfer and filling operations.

In embodiments, said pharmaceutical composition has a pH that is about 6.5 to about 7.5, about 6.5 to about 7.0, about 7.0 to about 7.5, about 6.45 to about 6.55, about 6.55 to about 6.65, about 6.65 to about 6.75, about 6.75 to about 6.85, about 6.85 to about 6.95, about 6.95 to about 7.05, about 7.05 to about 7.15, about 7.15 to about 7.25, about 7.25 to about 7.35, about 7.35 to about 7.45, or about 7.45 to about 7.55.

In embodiments, a pharmaceutical composition (e.g., a topical formulation to the eye) is isotonic or slightly hypotonic in order to combat any hypertonicity of tears caused by evaporation and/or disease. The compositions of the present invention generally have an osmolality in the range of 220-320 mOsm/kg, and preferably have an osmolality in the range of 235-260 mOsm/kg. The compositions of the invention have a pH in the range of about 5 to about 9 such as about 6.5 to about 7.5 or about 6.9 to about 7.4.

### Medical Implants

In some aspects, the invention features a medical implant comprising any pharmaceutical composition described herein.

In embodiments, said pharmaceutical composition is formulated for administration using a medical implant. In embodiments, said pharmaceutical composition is formulated for administration using an injectable medical implant. In embodiments, said pharmaceutical composition comprises a hydrogel. In embodiments, said hydrogel comprises one or more cellulosic polymers. In embodiments, said hydrogel comprises hydroxypropyl methylcellulose (HPMC), hydroxypropyl cellulose (HPC), polycarbophil, or xanthan gum. In embodiments, said hydrogel comprises any combination of hydroxypropyl methylcellulose (HPMC), hydroxypropyl cellulose (HPC), polycarbophil, or xanthan gum. In embodiments, said hydrogel comprises a copolymer of hydroxypropyl methylcellulose (HPMC), hydroxypropyl cellulose (HPC), polycarbophil, or xanthan gum.

In embodiments, a medical implant is injectable.

In embodiments, a medical implant (e.g., an injectable medical implant) is an ophthalmic medical implant.

In embodiments, an injectable medical implant is an ophthalmic medical implant.

In embodiments, an ophthalmic medical implant is a glaucoma drainage device.

In embodiments, an ophthalmic medical implant comprises silicone, polypropylene, a biocompatible metal, or collagen. In embodiments, an ophthalmic medical implant comprises silicone. In embodiments, an ophthalmic medical implant comprises polypropylene. In embodiments, an ophthalmic medical implant comprises a biocompatible metal (e.g., stainless steel, titanium, or gold). In embodiments, an ophthalmic medical implant comprises collagen (e.g., a collagen matrix).

In embodiments, an ophthalmic medical implant is a stent. In embodiments, an ophthalmic medical implant is a gel stent.

In embodiments, an ophthalmic medical implant is a shunt implant. In embodiments, an ophthalmic medical implant is a tube shunt. In embodiments, an ophthalmic medical implant is a shunt implant. In embodiments, an ophthalmic medical implant is a soft shunt implant.

In embodiments, an ophthalmic medical implant is a glaucoma valve.

In embodiments, an ophthalmic medical implant is a drainage tube.

In embodiments, an ophthalmic medical implant is used to lower intraocular pressure (IOP) in a subject in need thereof.

In embodiments, an ophthalmic medical implant is an intraocular implant.

In embodiments, an ophthalmic medical implant is a depot implant.

In embodiments, a medical implant (e.g., an ophthalmic medical implant) is biodegradable.

In embodiments, a medical implant (e.g., an ophthalmic medical implant) comprises a polymer.

In embodiments, a medical implant (e.g., an ophthalmic medical implant) comprises a biodegradable polymer.

In embodiments, a medical implant (e.g., an ophthalmic medical implant) comprises a mixture of biodegradable polymers.

In embodiments, a medical implant (e.g., an injectable medical implant) comprises a polyester biodegradable polymer. In embodiments, a medical implant (e.g., an injectable medical implant) comprises a mixture of polyester biodegradable polymers.

In embodiments a medical implant (e.g., an injectable medical implant) comprises a polyester biodegradable polymer that is polylactic acid (PLA) or poly(lactic-co-glycolic acid) (PLGA).

In embodiments a medical implant (e.g., an injectable medical implant) comprises a polyester biodegradable polymer that is a copolymer of polylactic acid (PLA) or poly(lactic-co-glycolic acid) (PLGA).

In embodiments a medical implant (e.g., an injectable medical implant) comprises a mixture of polyester biodegradable polymers (e.g., a mixture comprising polylactic acid (PLA) and/or poly(lactic-co-glycolic acid) (PLGA)).

In embodiments, a medical implant (e.g., an ophthalmic medical implant) comprises a hydrogel.

In embodiments, a hydrogel is biodegradable.

In embodiments, a hydrogel comprises polyethylene glycol.

In embodiments, a hydrogel comprises one or more cellulosic polymers.

In embodiments, said hydrogel comprises hydroxypropyl methylcellulose (HPMC), hydroxypropyl cellulose (HPC), polycarbophil, or xanthan gum. In embodiments, said hydrogel comprises any combination of hydroxypropyl methylcellulose (HPMC), hydroxypropyl cellulose (HPC), polycarbophil, or xanthan gum. In embodiments, said hydrogel comprises a copolymer of hydroxypropyl methylcellulose (HPMC), hydroxypropyl cellulose (HPC), polycarbophil, or xanthan gum.

In embodiments, a medical implant (e.g., an injectable medical implant) releases the pharmaceutical composition for a period that is about 3-24 months, about 3-18 months, about 3-15 months, about 3-12 months, about 3-6 months, about 4-15 months, about 4-12 months, about 5-15 months, about 5-12 months, about 6-15 months, or about 6-12 months.

### Routes of Administration

Administration of a compound, composition, or pharmaceutical composition as described herein can be by any method known to those skilled in the art.

In embodiments, administration is via local administration.

Exemplary local administration routes to the eye include: topical, subconjunctival, periocular, retrobulbar, subtenon, intracameral, intravitreal, intraocular, subretinal, juxtascleral and suprachoroidal administration.

In embodiments, administration is topical administration.

Systemic or parenteral administration may be feasible including intravenous, subcutaneous, intramuscular and oral delivery.

In embodiments, a method of administration is intravitreal or subtenon injection of solutions or suspensions, or intravitreal or subtenon placement of bioerodible or non-bioerodible devices, or by topical ocular administration of solutions or suspensions, or posterior juxtascleral administration of a gel formulation.

In embodiments, a method of administration is via injection (e.g., via depot injection).

### Methods of Treating and Preventing Diseases

The present invention is also directed to methods of treating or preventing diseases in a subject comprising administering a compound or composition (e.g., a pharmaceutical composition) as described herein. Administering a compound or composition described herein can result in a beneficial therapeutic effect (e.g., lowering intraocular pressure in a patient in need thereof).

A subject may be a mammal, such as a human, a primate, a cow, a horse, a dog, a cat, a mouse, or a rat. In embodiments, a subject is a human.

In some aspects, the present invention is directed to methods of treating or preventing an ophthalmic disease in a subject comprising administering to the subject an effective amount of a compound or composition (e.g., a pharmaceutical composition) as described herein.

In embodiments, an ophthalmic disease is glaucoma, elevated intraocular pressure or ocular hypertension.

In some aspects, the invention features a method of treating glaucoma in a patient in need thereof, said method comprising administering to said patient an effective amount of any compound or composition (e.g., a pharmaceutical composition) described herein.

The glaucoma may be any type of glaucoma, such as primary open angle glaucoma, angle closure glaucoma, normal tension glaucoma, congenital glaucoma, neovascular glaucoma, steroid-induced glaucoma, or glaucoma related to ocular trauma (e.g., ghost cell glaucoma or glaucoma related to choroidal detachment).

In embodiments, a compound or composition (e.g., a pharmaceutical composition) described herein will lower intraocular pressure (e.g., intraocular pressure associated with glaucoma).

In embodiments, a pharmaceutical composition comprising a linear peptidic NPR-B agonist (e.g., a compound of Formula (B) such as SEQ ID NO:1) and a prostaglandin agonist (e.g., latanoprost) will significantly lower intraocular pressure (e.g., intraocular pressure associated with glaucoma) compared to when either the linear peptidic NPR-B agonist or prostaglandin agonist are administered alone.

In another aspect, the invention features method of lowering intraocular pressure in a patient in need thereof, said method comprising administering to said patient an effective amount of any compound or composition (e.g., a pharmaceutical composition) described herein. For example, the present invention is directed to methods of lowering intraocular pressure in a subject, comprising administering to the subject a compound or composition (e.g., a pharmaceutical composition) described herein wherein intraocular pressure is lowered. In embodiments, the subject is a human. In embodiments, the human is a patient with ocular hypertension or elevated IOP.

It is noted that some individuals with an elevated IOP may not develop glaucoma and are considered to have ocular hypertension.

In embodiments of the present invention, a subject is receiving one or more additional forms of therapy directed to treatment or prevention of eye disease such as the ophthalmic diseases described herein.

In embodiments, a compound or composition (e.g., a pharmaceutical composition) described herein may be administered along with another agent or therapeutic method directed to treatment or prevention of eye disease. For example, administration of a compound or composition (e.g., a pharmaceutical composition) described herein to a human subject may precede, follow, or be concurrent with other therapies for glaucoma, elevated intraocular pressure or ocular hypertension. In embodiments, a compound or composition (e.g., a pharmaceutical composition) described herein is formulated in the same composition as the secondary form of therapy. In embodiments, a compound or composition (e.g., a pharmaceutical composition) described herein is formulated separately from the secondary form of therapy. One of ordinary skill in the art would be familiar with protocols for administering more than one form of pharmacological therapy to a subject with a disease, and would be familiar with methods of formulating more than one pharmacological agent in the same composition.

Examples of secondary therapeutic agents include: anti-glaucoma agents, such as beta-blockers including timolol, betaxolol, levobetaxolol, carteolol; miotics including pilocarpine; carbonic anhydrase inhibitors; prostaglandins; seretonergics; muscarinics; dopaminergic agonists; adrenergic agonists including apraclonidine and brimonidine; anti-angiogenesis agents; anti-infective agents including quinolones such as ciprofloxacin, and aminoglycosides such as tobramycin and gentamicin; non-steroidal and steroidal anti-inflammatory agents, such as suprofen, diclofenac, ketorolac, rimexolone and tetrahydrocortisol; growth factors, such as nerve growth factor (NGF), basic fibroblast growth factor (bFGF), brain-derived neurotrophic factor (BDNF), ciliary neutrophic factor (CNTF); immunosuppressant agents; and anti-allergic agents including olopatadine. Information pertaining to olopatadine formulations can be found in U.S. Pat. No. 6,995,186, U.S. Patent App. Pub. No. 2005/0158387, and U.S. Patent App. Pub. No. 2003/0055102, each of which is hereby specifically incorporated by reference. The ophthalmic drug may be present in the form of a pharmaceutically acceptable salt, such as timolol maleate, brimonidine tartrate or sodium diclofenac.

Other examples of a secondary therapeutic agent include a receptor tyrosine kinase (RTK) inhibitor. Exemplary RTK inhibitors are described in U.S. Patent App. Pub. No. 2006/0189608, and U.S. Pat. No. 7,297,709, both of which are hereby specifically incorporated by reference. In preferred embodiments, the receptor tyrosine kinase inhibitor is N-[4-[3-amino-1H-indazol-4-yl]phenyl]-N′-(2-fluoro-5-methylphenyl)urea.

In embodiments, a secondary therapeutic agent is a steroid. For example, the steroid may be a glucocorticoid, a progestin, a mineralocorticoid, or a corticosteroid. Exemplary corticosteroids include cortisone, hydrocortisone, prednisone, prednisolone, methylprednisone, triamcinolone, fluoromethalone, dexamethasone, medrysone, betamethasone, loteprednol, fluocinolone, flumethasone, or mometasone. Other examples of steroids include androgens, such as testosterone, methyltestosterone, or danazol. The secondary therapeutic agent may also be a glucocorticoid that is devoid of typical glucocorticoid side-effects, such as a cortisene. Preferred cortisenes for use in the methods of the invention include anecortave acetate and anecortave desacetate. Often steroids are administered as ester, acetal, or ketal prodrugs, many of which are water-insoluble. The secondary therapeutic agents may be directed to treatment or prevention of a single disease, or can be directed to treatment or prevention of two or more diseases.

In addition to pharmacological agents, surgical procedures can be performed in combination with the administration of a compound or composition (e.g., a pharmaceutical composition) described herein.

One such surgical procedure can include laser trabeculoplasty or trabeculectomy. In laser trabeculoplasty, energy from a laser is applied to a number of noncontiguous spots in the trabecular meshwork. It is believed that the laser energy stimulates the metabolism of the trabecular cells, and changes the extracellular material in the trabecular meshwork.

Another surgical procedure may include filtering surgery. With filtering surgery, a hole is made in the sclera near the angle. This hole allows the aqueous fluid to leave the eye through an alternate route. The most commonly performed filtering procedure is a trabeculectomy. In a trabeculectomy, a conjunctiva incision is made, the conjunctiva being the transparent tissue that covers the sclera. The conjunctiva is moved aside, exposing the sclera at the limbus. A partial thickness scleral flap is made and dissected half-thickness into the cornea. The anterior chamber is entered beneath the scleral flap and a section of deep sclera and/or trabecular meshwork is excised. The scleral flap is loosely sewn back into place. The conjunctival incision is tightly closed. Post-operatively, the aqueous fluid passes through the hole, beneath the scleral flap which offers some resistance and collects in an elevated space beneath the conjunctiva called a bleb. The fluid then is either absorbed through blood vessels in the conjunctiva or traverses across the conjunctiva into the tear film.

In embodiments, a compound (e.g., a compound of any one of Formulas (I)-(V) such as Compound 1) or combination (e.g., a composition comprising a first compound that is a linear peptidic NPR-B agonist, or a pharmaceutically acceptable salt thereof; and a second compound that is a prostaglandin agonist, or a pharmaceutically acceptable salt thereof) described herein can result in synergistic therapeutic effects.

Methods of analyzing synergy of combinations of therapeutic agents are known in the art (e.g., statistical analyses).

The compounds and compositions described herein can provide certain improvements, e.g., improved therapeutic effects.

For example, an improved therapeutic effect may be observed when compared to a therapeutic effect obtained from administration of a single therapeutic agent or a therapeutic effect that is additive of multiple therapeutic agents.

In embodiments, an improved therapeutic effect may be observed when compared to a therapeutic effect obtained from administration of a linear peptide NPR-B agonist (e.g., a peptide as described in, e.g., U.S. Pat. No. 8,546,523 or International Publication No. WO 2011/038061 or a peptide such as that according to SEQ ID NO:1 described herein).

In embodiments, an improved therapeutic effect may be observed when compared to a therapeutic effect obtained from administration of a prostaglandin agonist (e.g., latanoprost).

In embodiments, an improved therapeutic effect may be observed when compared to the calculated additive therapeutic effects obtained from, e.g., independent administration of a linear peptide NPR-B agonist and a prostaglandin agonist.

In embodiments, an improved effect is observed for reduction of intraocular pressure.

In embodiments, an improved effect is demonstrated by a therapeutic effect resulting from the administration of one or more of the combined therapeutic agents (e.g., a linear peptide NPR-B agonist and a prostaglandin agonist) at a lower dosage (e.g., a lower molar amount) than the dosage that is effective when said therapeutic agent is administered as a monotherapy. In embodiments, a linear peptide NPR-B agonist (e.g., a peptide as described in, e.g., U.S. Pat. No. 8,546,523 or International Publication No. WO 2011/038061 or a peptide such as that according to SEQ ID NO:1 described herein) is used in a lower dosage than the effective dosage used in monotherapy. In embodiments, a prostaglandin agonist (e.g., latanoprost) is used in a lower dosage than the effective dosage used in monotherapy.

In embodiments, an improved effect is demonstrated by a therapeutic effect resulting from the administration of one or more of the combined therapeutic agents (e.g., a linear peptide NPR-B agonist and a prostaglandin agonist) at a frequency that is less than the frequency of administration commonly used when said therapeutic agent is administered as a monotherapy. In embodiments, a linear peptide NPR-B agonist (e.g., a peptide as described in, e.g., U.S. Pat. No. 8,546,523 or International Publication No. WO 2011/038061 or a peptide such as that according to SEQ ID NO:1 described herein) is administered once daily.

### EXAMPLES

### Listing of Abbreviations

IOP intraocular Pressure
HPMC hydroxypropyl methylcellulose
BAK benzalkonium chloride
NaCl sodium chloride
Polysorbate 80 polyoxyethylenesorbitan monooleate
EDTA ethylenediaminetetraacetic acid
PBS phosphate buffered saline
HPC hydroxypropyl cellulose
POAG primary open angle glaucoma

### Example 1. Compounds

Compounds can be prepared according to methods well known in the art. Exemplary linear peptide NPR-B agonists described herein can be prepared according to the methods described in U.S. Pat. No. 8,546,523. Prostaglandin agonists (e.g., latanoprost, travoprost, or bimatoprost) can be prepared according to methods known in the art or obtained from commercial sources.

### Example 2. Exemplary Compositions Comprising Single Active Agents

Exemplary compositions comprising a single active agent are described herein. These exemplary compositions are suitable for use in any of the methods described herein or for preparation of any of the compositions described herein. Amounts of any component specified in the exemplary compositions can be varied (e.g., the amount of a component can be varied up to about 20%, up to about 15%, up to about 10%, or up to about 5% in either direction).

An exemplary pharmaceutical composition comprising a linear peptidic NPR-B agonist is provided in Table 1. Each individual component of the exemplary pharmaceutical composition can be varied. For example, a pharmaceutical composition comprising about 0.03% (w/w) of SEQ ID NO:1 can be prepared.

TABLE 1
Exemplary pharmaceutical composition comprising SEQ ID NO: 1

| | | Amount |
|---|---|---|
| | | (%(w/w) unless |
| Component | | otherwise noted) |
| linear peptidic NPR-B agonist | SEQ ID NO: 1 | 0.03%-0.60% |
| viscosity enhancing agent | HPMC | 0.50% |
| Preservative | BAK | 0.01% |
| chelating agent | EDTA | 0.01% |
| Surfactant | Polysorbate 80 | 0.05% |
| buffering agent | Phosphate buffer | 0.20% |
| tonicity agent | NaCl | 0.75% |

| | |
|---|---|
| Target Final pH | 7.4 |

Exemplary pharmaceutical compositions comprising a prostaglandin agonist are described in Table 2 and Table 3.

TABLE 2
Exemplary pharmaceutical composition comprising latanoprost

| | | Amount |
|---|---|---|
| | | (%(w/w) unless |
| Component | | otherwise noted) |
| prostaglandin agonist | latanoprost | 0.0025-0.001% |
| preservative | BAK | 0.02% |
| buffering agent | sodium phosphate | 0.135M |

| | |
|---|---|
| Target Final pH | 7.0 |

TABLE 3
Exemplary pharmaceutical composition comprising travoprost
Components

| prostaglandin agonist | travoprost |
|---|---|
| surfactant | polyoxyl 40 hydrogenated castor oil |
| preservative | boric acid |
| solubility enhancing agent | propylene glycol |
| tonicity agent | sorbitol |
| buffering/stabilizing agent | zinc chloride |
| pH adjusting agent | sodium hydroxide |
| pH adjusting agent | hydrochloric acid |
| Target Final pH | 5.7 |

An exemplary pharmaceutical composition comprising a β-adrenergic antagonist is provided in Table 4.

TABLE 4
Exemplary pharmaceutical composition comprising timolol
Components

| β-adrenergic antagonist | timolol |
|---|---|
| preservative | benzalkonium chloride |
| buffering agent | dibasic sodium phosphate |
| buffering agent | monobasic sodium phosphate |
| pH adjusting agent | hydrochloric acid |
| Target Final pH | 7.0 |

### Example 3. Exemplary Composition Comprising a Linear Peptidic NPR-B Agonist and a Prostaglandin Agonist

An exemplary composition comprising multiple active agents is described herein. This exemplary composition is suitable for use in any of the methods described herein or for preparation of any of the compositions described herein. Amounts of any component specified in the exemplary composition can be varied (e.g., the amount of a component can be varied up to about 20%, up to about 15%, up to about 10%, or up to about 5% in either direction).

An exemplary pharmaceutical composition comprising a linear peptidic NPR-B agonist and a prostaglandin agonist is provided in Table 5.

TABLE 5
Exemplary pharmaceutical composition comprising a linear
peptidic NPR-B agonist and a prostaglandin agonist

| | | Amount |
|---|---|---|
| | | (%(w/w) unless |
| Component | | otherwise noted) |
| linear peptidic NPR-B agonist | SEQ ID NO: 1 | 0.10% |
| prostaglandin agonist | latanoprost | 0.005% |
| viscosity enhancing agent | HPMC (E4M) | 0.50% |
| surfactant | Polysorbate 80 | 0.20% |
| buffering agent | Phosphate buffer | 0.20% |
| tonicity agent | NaCl | 0.75% |

| | |
|---|---|
| Target Final pH | 7.0 |

### Example 4. Activity Assays

Combination Study of Topical Application of Latanoprost and an NPR-B Agonist Peptide (SEQ ID NO:1).

A combination study of the therapeutic effects of latanoprost and NPR-B agonist peptide (SEQ ID NO:1) was undertaken in normotensive Dutch-Belted rabbits. The treatment group was selected based on latanoprost challenge: rabbits that were latanoprost responders were randomized into latanoprost-only treatments and combination therapy comprising latanoprost and SEQ ID NO:1.

Treatment groups received one of the following: vehicle only; latanoprost (0.005% only); SEQ ID NO:1 (30 μg; 0.1%); or a combination of latanoprost (0.005% only) and SEQ ID NO:1 (30 μg; 0.1%), where latanoprost was administered five minutes prior to SEQ ID NO:1.

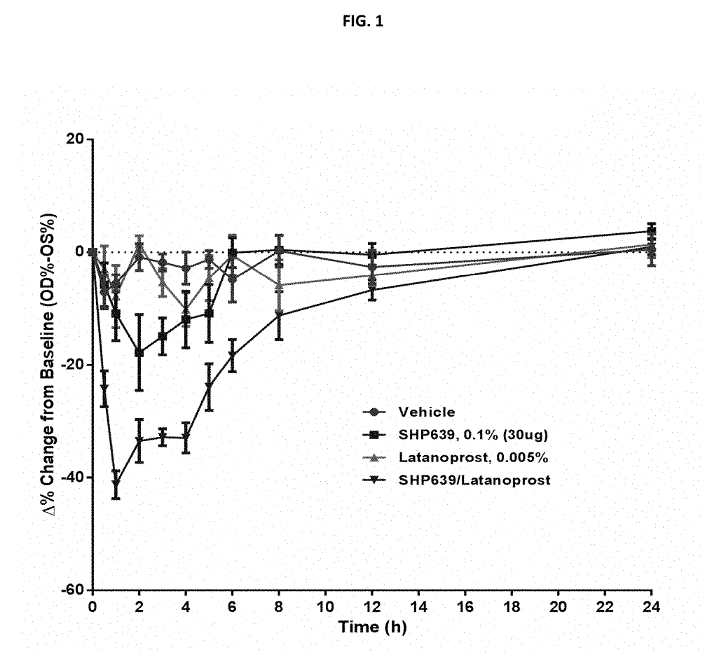

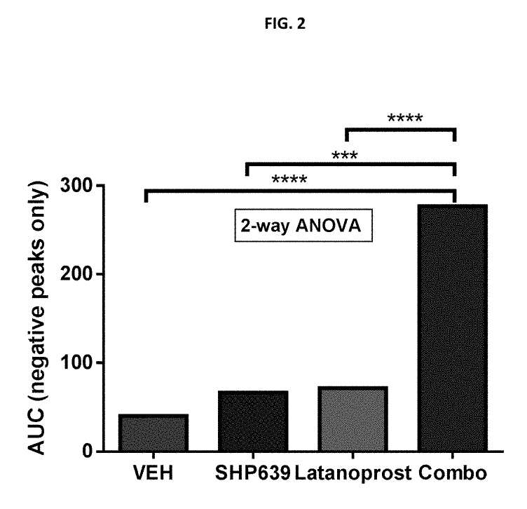

Intraocular pressure (IOP) was measured at time points: pre-dose, 0.5 h, 1 h, 2 h, 3 h, 4 h, 5 h, 6 h, 8 h, 12 h, and 24 h (FIG. 1). The greatest decrease in IOP was observed using the combination therapy of latanoprost and SEQ ID NO:1, where a synergistic effect was observed based on area under the curve (AUC) values (FIG. 2).

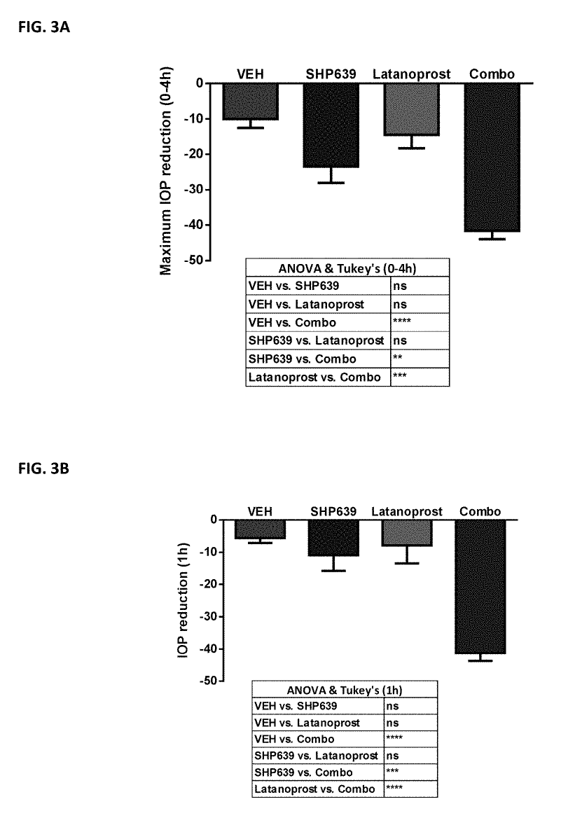

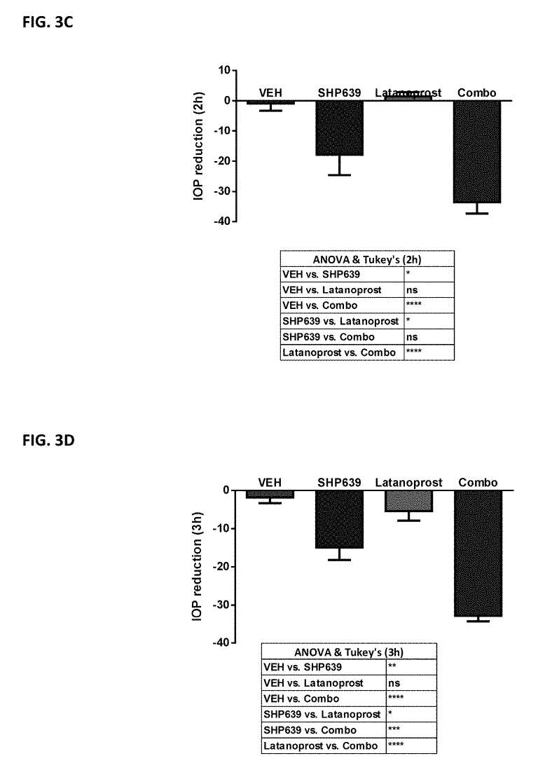

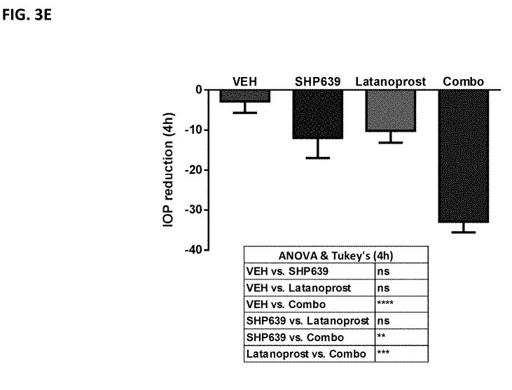

Synergistic effects were also confirmed by analysis of maximum IOP reduction over 0-4 hours: FIGS. 3A-3E show maximum IOP reduction and statistical analyses (ANOVA and Tukey's) based on the maximum IOP reduction observed using (1) vehicle; (2) SEQ ID NO:1; (3) latanoprost; or (4) latanoprost and SEQ ID NO:1, as measured over 0-4 hours.

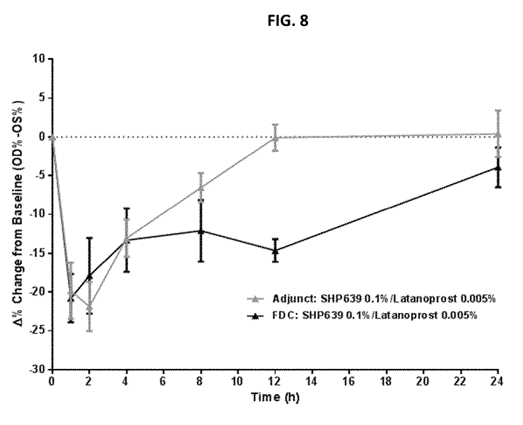

As shown in FIG. 8 the maximum IOP reduction for the adjunct dosing of SEQ ID NO:1 0.1%/latanoprost 0.005% administered as two separate drops five minutes apart (preserved with BAK) was similar to the fixed-dose combination formulation (i.e both agents in same bottle) without BAK (i.e preservative free). FIG. 8 further demonstrates that the duration of the fixed-dose combination formulation is longer compared to the adjunct formulation.

### Example 5. Pharmacodynamics of Topical Application of Latanoprost and an NPR-B Agonist Peptide (SEQ ID NO:1)

A study of the pharmacodynamics following a single ascending topical ocular dose of SEQ ID NO:1; latanoprost; or a latanoprost/SEQ ID NO:1 combination was undertaken in normotensive dogs.

Dose Administration

Animals were not fasted prior to dose administration. As appropriate, latanoprost was administered first followed by SEQ ID NO:1 approximately 5 minutes later. The topical ocular dose (35 μL) was administered to the central or superior part of the cornea via a micropipette and allowed to spread across the surface of the eye (Deviation). After the dose was administered, the eye was allowed to close naturally. Each animal was then restrained for approximately 1 minute to prevent rubbing of the eyes. The right eye was dosed first. All measurement and collection times were based on the time of dosing of the second (left) eye.

Intraocular Pressure (IOP) Preconditioning

Following arrival, animals were preconditioned (trained) to IOP measurement procedures three times daily (i.e. 07:00, 12:00, and 15:00±0.5 hours) at least three times/week for approximately seven weeks prior to Phase 1. Three readings/eye were taken using a TonoVet.

Intraocular Pressure (IOP) Measurement

Intraocular pressure was measured at −1, 0 (predose), 1, 2, 4, 8, 12, 24, and 48 hours postdose (following the last dose, as applicable) for all animals in Group 1, Phases 1 through 10. Three readings/eye were taken using a TonoVet.

Pharmacodynamics Study

Animals selected for pharmacodynamics (Group 1) were administered SEQ ID NO:1 (12, 35, 105, or 210 μg/eye), latanoprost (0.35, 0.875, or 1.75 μg/eye), and latanoprost/SEQ ID NO:1 in combination to the right eye (OD) and vehicle or phosphate-buffered saline (PBS) to the left eye (OS).

Intraocular pressure (IOP) was measured at various time points from pre-dose to 48 hours post-dose.

Pharmacodynamic animals (Group 1) were unilaterally treated with a single topical ocular dose of latanoprost (0.001, 0.0025, and 0.005%), followed by approximately 5 minutes later with a dose of SEQ ID NO:1 0.03%, in the right eye (OD) only.

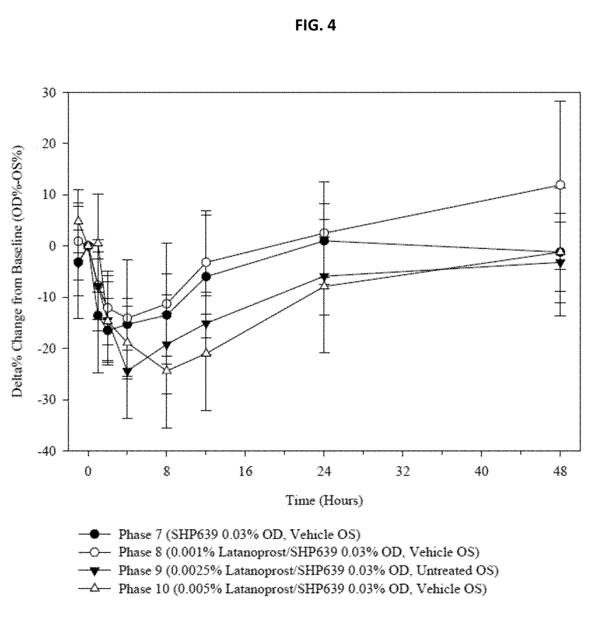

Following dosing of SEQ ID NO:1 (0.03%) and latanoprost at three concentrations, the mean delta IOP percentages decreased with the maximal effect (Emax) observed at 4 to 8 hours post-dose (TEmax) (FIG. 4).

As shown in FIG. 4, a latanoprost dose response in combination with SEQ ID NO:1 was observed as the maximal delta decrease (Emax) in IOP was greater with increasing latanoprost concentrations, in combination with SEQ ID NO:1.

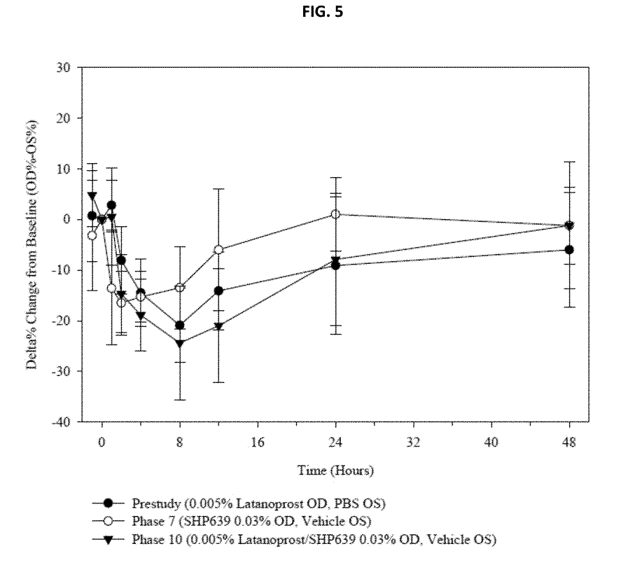

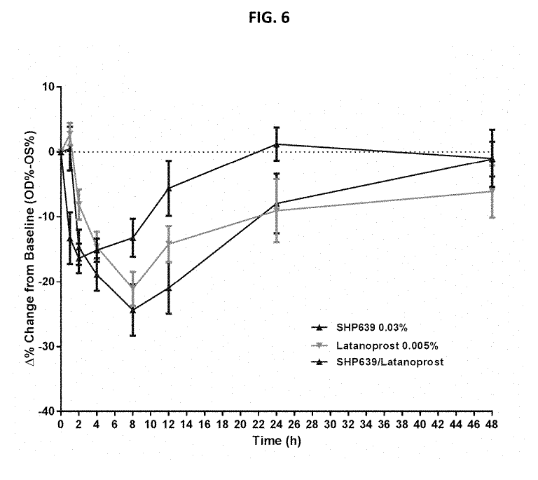

A synergistic effect was observed as the combination of latanoprost/SEQ ID NO:1 yielded a greater reduction in mean delta IOP compared to either latanoprost or SEQ ID NO:1 alone (FIG. 5).

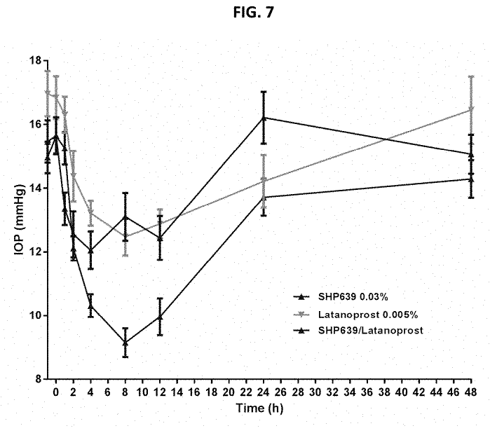

When SEQ ID NO:1 and latanoprost were administered adjunctively to female dogs the intraocular pressure was lower (7-9 mmHg) compared to when SEQ ID NO:1 and latanoprost were administered alone (12 mmHg) (FIG. 7).

As shown in FIG. 6 there was a significant improvement in reduction of intraocular pressure when SEQ ID NO:1 was administered as adjunct to latanoprost. The maximum percent change in intraocular pressure compared to contralateral eye when 0.03% SEQ ID NO:1 was administered alone was 16% and when 0.005% Lantanoprost was administered alone was 21%. A synergistic effect was observed as the adjunctive administration of SEQ ID NO:1 and latanoprost yielded a maximum percent change in intraocular pressure compared to contralateral eye of 24%.

### Example 6. Pharmacodynamics of Adjunct Therapy of Latanoprost and an NPR-B Agonist Peptide (SEQ ID NO:1) Compared to Fixed-Dose Combination Formulation

A study of the pharmacodynamics following adjunct therapy of SEQ ID NO:1 and latanoprost or a or a fixed-dose combination of SEQ ID NO:1 and latanoprost was undertaken in normotensive beagle dogs.

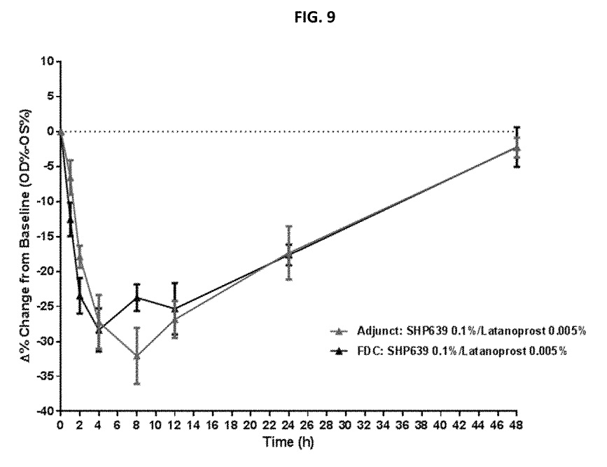

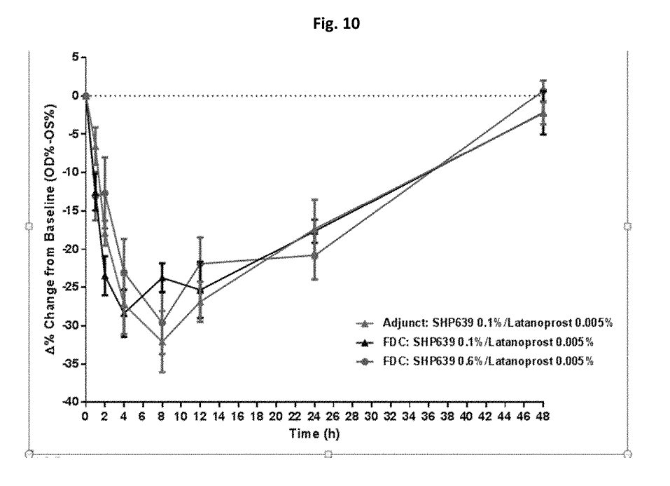

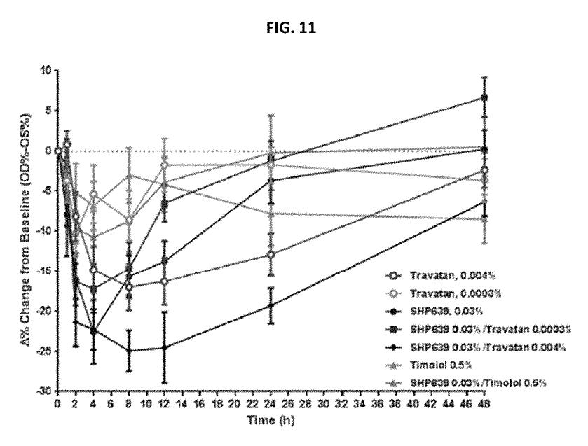

Intraocular pressure (IOP) was measured at time points: 1 h, 2 h, 3 h, 4 h, 5 h, 6 h, 8 h, 12 h, 24 h, and 48 hours post-administration (FIG. 9 and FIG. 10).

As presented in FIG. 9 and FIG. 10 there is no difference in IOP reduction with adjunct administration (contains BAK) compared to fixed-dose combination administration (BAK removed) of SEQ ID NO:1 and latanoprost, therefore the data suggest that BAK does not affect drug absorption in dog.

As shown in FIG. 10 there was no difference in IOP reduction when the concentration of SEQ ID NO:1 was increased from 0.1% to 0.6% for the fixed-dose formulation.

### Example 7. Adjunctive Therapy with Travoprost and Timolol

Additional studies were undertaken relating to adjunctive therapy using SEQ ID NO:1 in combination with travoprost (Travatan Z®) and timolol.

Eight female normotensive beagle dogs were used in a crossover study design, with the following treatment groups:

1. 0.035% SEQ ID NO:1;

2. 0.0003% travoprost;

3. 0.004% travoprost;

4. 0.5% timolol;

5. 0.03% SEQ ID NO:1 in combination with 0.0003% travoprost or 0.004% travoprost; and

6. 0.03% SEQ ID NO:1 in combination with timolol.

The travoprost and timolol were dosed about five minutes prior to SEQ ID NO:1. Exposure in AH were evaluated in different dogs.

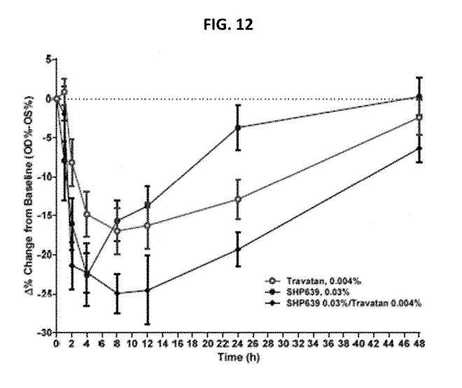

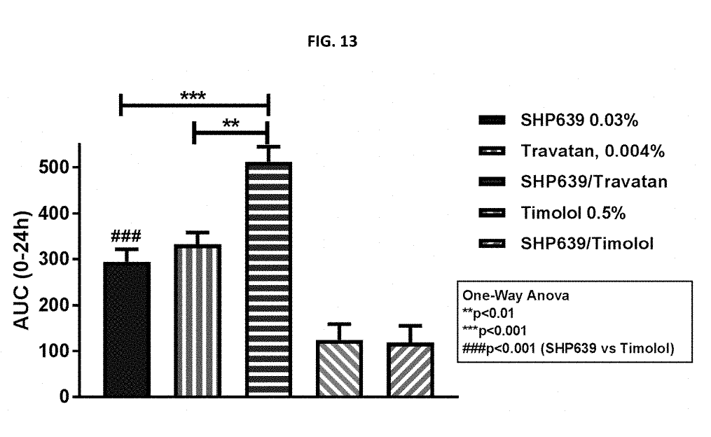

FIG. 11 shows the effects on IOP reduction in dogs using adjunctive therapy with either travoprost (Travatan Z®) and timolol, where the percent change in IOP was determined in comparison to the contralateral eye. In particular, a significantly improved IOP reduction was observed using the combination of SEQ ID NO:1 and travoprost, which is also evident in FIG. 12. Additionally, there is a prolonged duration observed with the combination of SEQ ID NO:1 and travoprost as compared to monotherapy (FIG. 13)

All publications and patent applications cited in this specification are hereby incorporated by reference herein in their entireties as if each individual publication or patent application were specifically and individually indicated as being incorporated by reference and as if each reference was fully set forth in its entirety. To the extent that there is any conflict between any publication or patent application incorporated herein, the present specification controls. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of treating an ophthalmic disease in a patient, said method comprising co-administering to said patient an effective amount of
a first compound that is a linear peptidic NPR-B agonist; and
a second compound that is a prostaglandin agonist or a β-adrenergic antagonist.

2. The method of claim 1, wherein said first compound is a compound of formula (B-1), 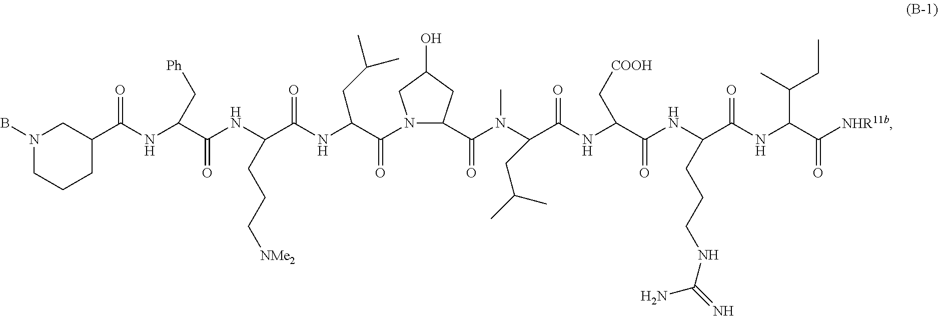
or a pharmaceutically acceptable salt thereof, wherein
B is selected from the group consisting of R^{b1}— and R^{b2}—C(O)—;
R^{b1} is selected from the group consisting of C₆-C₁₀ alkyl and C₅-C₁₀ alkyl substituted by NR^{b4}R^{b5};
R^{b2} is selected from the group consisting of C₅-C₁₀ alkyl and C₅-C₁₀ alkyl substituted by NR^{b4}R^{b5};
R^{b4} and R^{b5} are, independently, selected from the group consisting of H and C₁-C₄ alkyl; and
R^{11b} is selected from the group consisting of H, C₁-C₈ alkyl, C₄-C₈ cycloalkyl, C₇-C₁₂ bicycloalkyl, and C₁-C₄ alkyl-C₄-C₈ cycloalkyl.

3. The method of claim 1, wherein said first compound is Occ-Sni-Phe-orn(Me2)-Leu-Hyp-Nml-Asp-Arg-Ile-NH₂ (SEQ ID NO:1), or a pharmaceutically acceptable salt thereof.

4.-5. (canceled)

6. The method of claim 1, wherein said second compound is a prostaglandin agonist that is latanoprost, bimatoprost, travoprost, or tafluprost or a β-adrenergic antagonist that is betaxolol, carteolol, levobunolol, metipranolol, or timolol.

7.-8. (canceled)

9. The method of claim 1, wherein the ophthalmic disease is glaucoma, elevated intraocular pressure or ocular hypertension.

10. The method of claim 1, wherein said method comprises lowering intraocular pressure in a patient in need thereof.

11.-23. (canceled)

24. The method of claim 9, wherein said glaucoma is primary open angle glaucoma, angle closure glaucoma, normal tension glaucoma, congenital glaucoma, neovascular glaucoma, steroid-induced glaucoma, or glaucoma related to ocular trauma.

25.-28. (canceled)

29. The method of claim 1, wherein said single composition comprises:
said first compound in an amount that is about 0.01% (w/w) to about 0.75% (w/w);
said second compound in an amount that is about 0.0001% (w/w) to about 0.1% (w/w); and
a pharmaceutically acceptable excipient.

30.-44. (canceled)

45. The method of claim 1, wherein said first compound and/or said second compound is topically administered.

46.-50. (canceled)

51. A pharmaceutical composition comprising:
a first compound that is a linear peptidic NPR-β agonist;
a second compound that is a prostaglandin agonist or a β-adrenergic antagonist; and
a pharmaceutically acceptable excipient.

52. The pharmaceutical composition of claim 51, wherein said first compound is a compound of formula (B-1), 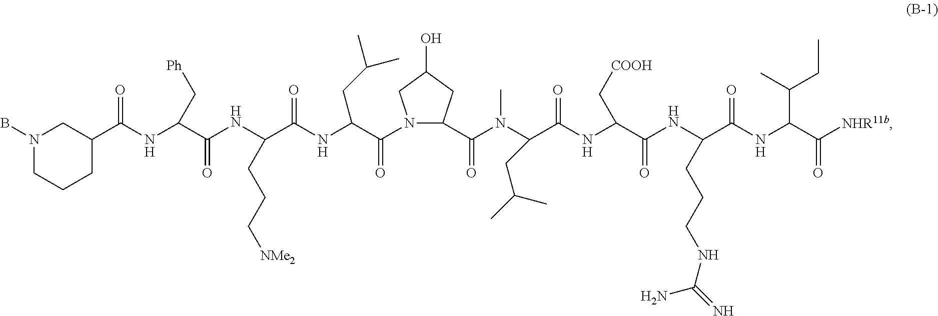
or a pharmaceutically acceptable salt thereof, wherein
B is selected from the group consisting of R^{b1}— and R^{b2}—C(O)—;
R^{b1} is selected from the group consisting of C₆-C₁₀ alkyl and C₆-C₁₀ alkyl substituted by NR^{b4}R^{b5};
R^{b2} is selected from the group consisting of C₆-C₁₀ alkyl and C₆-C₁₀ alkyl substituted by NR^{b4}R^{b5};
R^{b4} and R^{b5} are, independently, selected from the group consisting of H and C₁-C₄ alkyl; and
R^{11b} is selected from the group consisting of H, C₁-C₈ alkyl, C₄-C₈cycloalkyl, C₇-C₁₂bicycloalkyl, and C₁-C₄alkyl-C₄-C₈cycloalkyl.

53. The pharmaceutical composition of claim 51, wherein said first compound is Occ-Sni-Phe-orn(Me2)-Leu-Hyp-Nml-Asp-Arg-Ile-NH₂ (SEQ ID NO:1), or a pharmaceutically acceptable salt thereof.

54.-55. (canceled)

56. The pharmaceutical composition of claim 51, wherein said second compound is a prostaglandin agonist that is latanoprost, bimatoprost, travoprost, or tafluprost or a β-adrenergic antagonist that is betaxolol, carteolol, levobunolol, metipranolol, or timolol.

57.-60. (canceled)

61. The pharmaceutical composition of claim 51, wherein
said first compound, or a pharmaceutically acceptable salt thereof, is present in an amount that is about 0.01% (w/w) to about 0.15% (w/w); and
said second compound is present in an amount that is about 0.001% (w/w) to about 0.05% (w/w).

62.-64. (canceled)

65. The pharmaceutical composition of claim 51, wherein said pharmaceutical composition is formulated for ophthalmic use.

66. The pharmaceutical composition of claim 51, wherein said pharmaceutical composition is formulated for topical administration.

67.-76. (canceled)

77. A method of treating an ophthalmic disease in a patient, said method comprising administering to said patient an effective amount of the pharmaceutical composition of claim 51.

78. A method of lowering intraocular pressure in a patient in need thereof, said method comprising administering to said patient an effective amount of the pharmaceutical composition of claim 51.

79. A method of treating glaucoma in a patient in need thereof, said method comprising administering to said patient an effective amount of the pharmaceutical composition of claim 51.

80.-81. (canceled)

82. A compound that is:
a compound of formula (I), 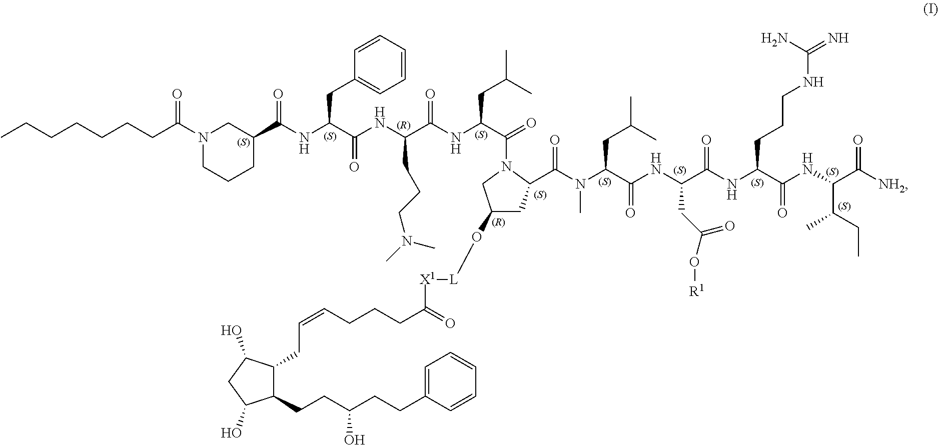
or a pharmaceutically acceptable salt thereof, wherein
X¹ is a covalent bond, —O—, —S—, or —NR^{X1}—,
R¹ is H, optionally substituted C₁-C₁₂ alkyl, or optionally substituted C₇-C₁₆ aralkyl;
R^{X1} is H or optionally substituted C₁-C₁₂ alkyl;
L represents a linker that is a covalent bond, optionally substituted C₁-C₁₂ alkylene, or optionally substituted 2- to 12-membered heteroalkylene, or
L represents a linker having the structure -(L¹)-L²-(L³)-, wherein
each of L¹ and L³ is independently a covalent bond, optionally substituted C₁-C₅ alkylene, or optionally substituted 2- to 6-membered heteroalkylene; and
L² is optionally substituted C₅-C₁₀ arylene or optionally substituted 5- to 10-membered heteroarylene;
or
a compound of formula (III), 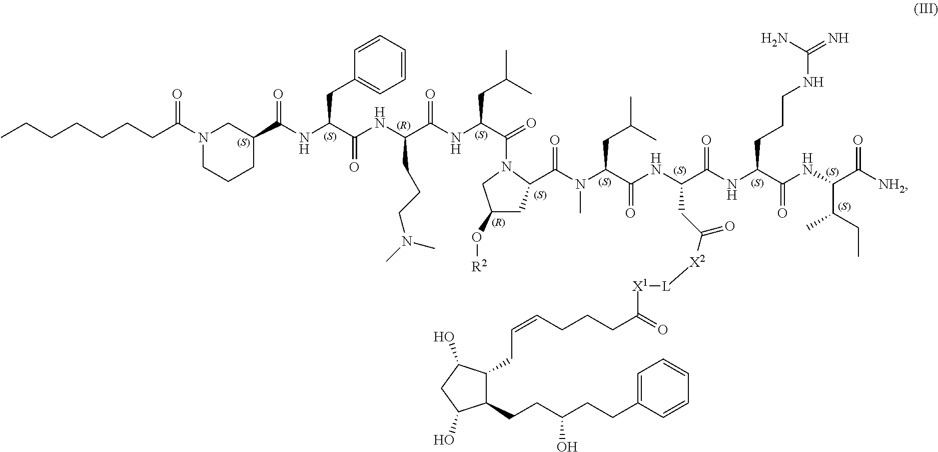
or a pharmaceutically acceptable salt thereof, wherein
R² is H, optionally substituted C₁-C₁₂ alkyl, or optionally substituted C₇-C₁₆ aralkyl;
X¹ is a covalent bond, —O—, —S—, or —NR^{X1}—,
X² is a covalent bond, —O—, —S—, or —NR^{X2}—,
each of R^{X1} and R^{X2} is independently H or optionally substituted C₁-C₁₂ alkyl;
L represents a linker that is a covalent bond, optionally substituted C₁-C₁₂ alkylene, or optionally substituted 2- to 12-membered heteroalkylene, or
L represents a linker having the structure -(L¹)-L²-(L³)-, wherein
each of L¹ and L³ is independently a covalent bond, optionally substituted C₁-C₅ alkylene, or optionally substituted 2- to 6-membered heteroalkylene; and
L² is optionally substituted C₅-C₁₀ arylene or optionally substituted 5- to 10-membered heteroarylene.

83.-92. (canceled)

93. The compound of claim 82, wherein said compound is of formula (II), 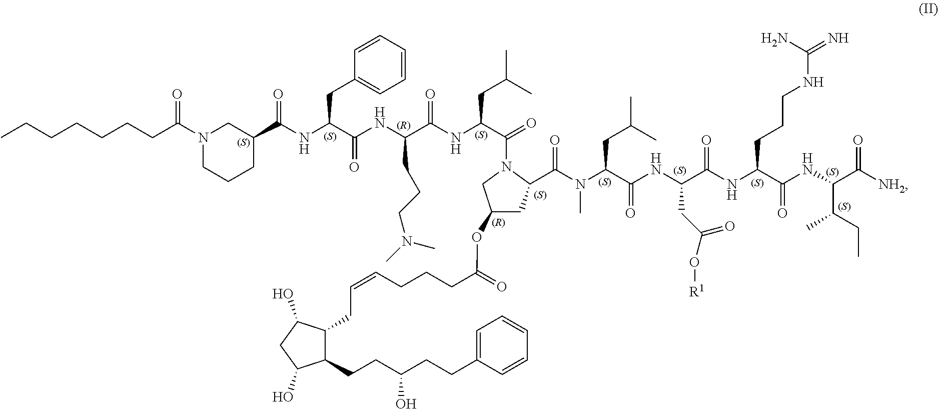
or a pharmaceutically acceptable salt thereof, wherein
R¹ is H, optionally substituted C₁-C₁₂ alkyl, or optionally substituted C₇-C₁₆ aralkyl.

94.-107. (canceled)

108. The compound of claim 82, wherein said compound is of formula (IV): 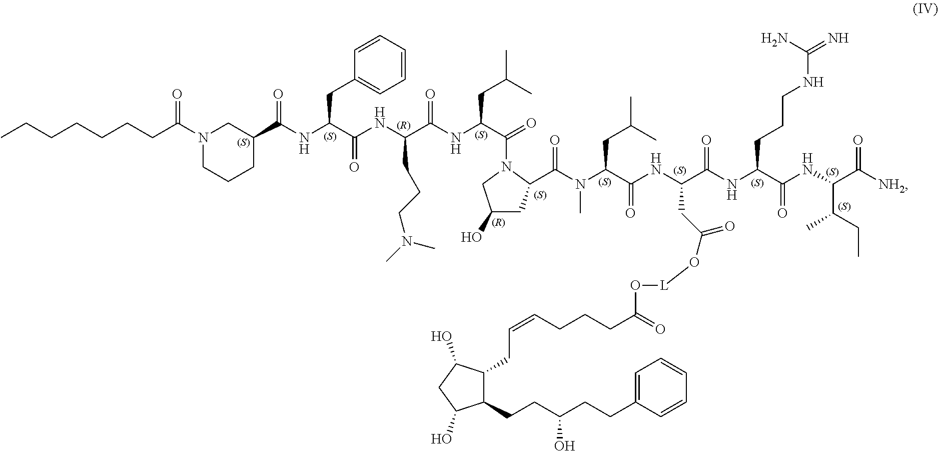
or a pharmaceutically acceptable salt thereof, wherein
L represents a linker that is optionally substituted C₁-C₁₂ alkylene or optionally substituted 2- to 12-membered heteroalkylene.

109. (canceled)

110. The compound of claim 82, having the following structure, 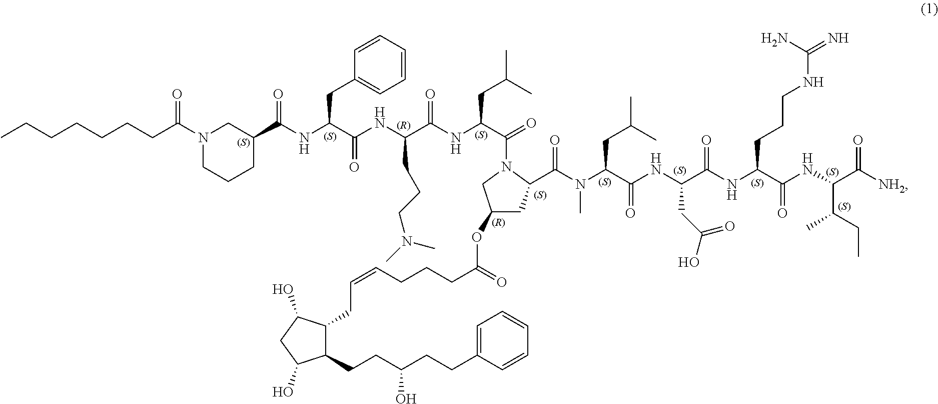
or a pharmaceutically acceptable salt thereof.

111. A pharmaceutical composition comprising
the compound of claim 82, and
a pharmaceutically acceptable excipient.

112. The pharmaceutical composition of claim 111, comprising the compound in an amount that is
about 0.001% (w/v) to about 1.000% (w/v), about 0.001% (w/v) to about 0.500% (w/v), about 0.001% (w/v) to about 0.250% (w/v), about 0.001% to about 0.150%, about 0.001% to about 0.100%, about 0.001% to about 0.090%, about 0.001% to about 0.075%, about 0.001% to about 0.050%, or about 0.001% to about 0.010%;
about 0.005% (w/v) to about 1.000% (w/v), about 0.005% (w/v) to about 0.500% (w/v), about 0.005% (w/v) to about 0.250% (w/v), about 0.005% to about 0.150%, about 0.005% to about 0.100%, about 0.005% to about 0.090%, about 0.005% to about 0.075%, about 0.005% to about 0.050%, or about 0.005% to about 0.010%;
about 0.010% (w/v) to about 2.000% (w/v), about 0.010% (w/v) to about 1.500% (w/v), about 0.010% (w/v) to about 1.000% (w/v), about 0.010% (w/v) to about 0.900% (w/v), about 0.010% (w/v) to about 0.800% (w/v), about 0.010% (w/v) to about 0.700% (w/v), about 0.010% (w/v) to about 0.600% (w/v), about 0.010% (w/v) to about 0.500% (w/v), about 0.010% (w/v) to about 0.250% (w/v), about 0.010% to about 0.150%, about 0.010% to about 0.100%, about 0.010% to about 0.090%, about 0.010% to about 0.075%, or about 0.010% to about 0.050%;
about 0.050% (w/v) to about 2.000% (w/v), about 0.050% (w/v) to about 1.500% (w/v), about 0.050% (w/v) to about 1.000% (w/v), about 0.050% (w/v) to about 0.500% (w/v), about 0.050% (w/v) to about 0.250% (w/v), about 0.050% (w/v) to about 0.200% (w/v), about 0.050% to about 0.150%, or about 0.050% to about 0.125%; or
about 0.075% (w/v) to about 2.000% (w/v), about 0.075% (w/v) to about 1.500% (w/v), about 0.075% (w/v) to about 1.250% (w/v), about 0.075% (w/v) to about 1.000% (w/v), about 0.075% (w/v) to about 0.750% (w/v), about 0.075% (w/v) to about 0.500% (w/v), about 0.075% (w/v) to about 0.250% (w/v), about 0.075% (w/v) to about 0.200% (w/v), or about 0.075% (w/v) to about 0.150% (w/v).

113. The pharmaceutical composition of claim 111, wherein said pharmaceutical composition is formulated for ophthalmic use.

114. The pharmaceutical composition of claim 111, wherein said pharmaceutical composition is formulated for topical administration.

115.-124. (canceled)

125. A method of treating an ophthalmic disease in a patient, said method comprising administering to said patient an effective amount of the pharmaceutical composition of claim 111.

126. A method of lowering intraocular pressure in a patient in need thereof, said method comprising administering to said patient an effective amount of the pharmaceutical composition of claim 111.

127. A method of treating glaucoma in a patient in need thereof, said method comprising administering to said patient an effective amount of the pharmaceutical composition of claim 111.

128.-129. (canceled)


SEQUENCE LISTING (1 sequence)

SEQ ID NO: 1 - protein, 10 aa, Artificial
  synthetic
  MISC_FEATURE (1)..(1): Xaa = Octanoic acid
  MISC_FEATURE (2)..(2): Xaa = (S)-nipecotic acid
  MISC_FEATURE (4)..(4): Xaa = (R)-2-amino-5-(dimethylamino)-pentanoic acid
  MISC_FEATURE (4)..(4): D form of amino acid at this position
  MISC_FEATURE (6)..(6): Xaa = (2S,4R)-4-hydroxypyrrolidine-2-carboxylic acid
  MISC_FEATURE (7)..(7): Xaa = (S)-N-methyl-leucine
XXFXLXXDRI